SEQUENCE LISTING (73 sequences)

SEQ ID NO: 1 - DNA, 117 bp, Artificial Aequence
  Oligonucleotide, 19aTAR (aka 19am)
ctcagatctgcctgcaactatgcaaaactaacagaggactgcaaacaaaaactagaaggaaatagcaggccaccatcagttttgcatagatttgcacaacggcagagagctcccagg

SEQ ID NO: 2 - DNA, 117 bp, Artificial Sequence
  Oligonucleotide, 19am3
gaaggaaatagcaggccaccatcagttttgcatagatttgcacaacttccagattccacctactgtggctggaaatgcaactatgcaaaactaacagaggactgcaaacaaaaacta

SEQ ID NO: 3 - DNA, 76 bp, Artificial Sequence
  Oligonucleotide, 19adcr1
ggaaaaatcagttttgcatagatttgcacaactacacacatgttgtagtgcaactatgcaagaccaaaaatcagaa

SEQ ID NO: 4 - DNA, 89 bp, Artificial Sequence
  Oligonucleotide, 122 (aka 122ENDO)
ttgcctagcagtagctatttagtgtgataatggcgtttgatagtttagacacaaacaccattgtcacactccacagctctgctaaggaa

SEQ ID NO: 5 - DNA, 124 bp, Artificial Sequence
  Oligonucleotide, 122TAR (aka 122m)
aggaaatagcctagcagtagctatttagtgtgataatggcgtttgatagggcagagagctcccaggctcagatctgcccacaaacaccattgtcacactccacagctctgctaaggaaacaaaa

SEQ ID NO: 6 - DNA, 71 bp, Artificial Sequence
  Oligonucleotide, 122s
ttgccctatttagtgtgataatggcgtttgatagtttagacacaaacaccattgtcacactccacagggaa

SEQ ID NO: 7 - DNA, 67 bp, Artificial Sequence
  Oligonucleotide, 221
acgaacacagaaatctacattgtatgccaggttcatgaaacccagcagacaatgtagctgttgccta

SEQ ID NO: 8 - DNA, 58 bp, Artificial Sequence
  Oligonucleotide, 143
aactgaccagagatgcagcactgcacctcttcctgagctacagtgcttcatctcagac

SEQ ID NO: 9 - DNA, 53 bp, Artificial Sequence
  Oligonucleotide, 15a
aatccacaaaccattatgtgctgctactttgcagcacaatatggcctgcacaa

SEQ ID NO: 10 - DNA, 57 bp, Artificial Sequence
  Oligonucleotde, 21
cagacagcccatcgactggtgttgacagtcaacatcagtctgataagctacccgaca

SEQ ID NO: 11 - DNA, 76 bp, Artificial Sequence; identical to SEQ ID NO: 3
  Oligonucleotde, 19aDcr1

SEQ ID NO: 12 - DNA, 82 bp, Artificial Sequence
  Oligonucleotide, 19aDcr3
gaaggaaaaatcagttttgcatagatttgcacaactacattcttcttgtagtgcaactatgcaaaactgcaaacaaaaacta

SEQ ID NO: 13 - DNA, 76 bp, Artificial Sequence
  Oligonucleotide, lin19a
accatcagttttgcatagatttgcacaactacaaaaatcagaaggaaaaagtagtgcaactatgcaaaactgacag

SEQ ID NO: 14 - DNA, 89 bp, Artificial Sequence
  Oligonucleotide, 122L
actccacagctctgctaaggaattgcctagcagtagctatttagtgtgataatggcgtttgatagtttagacacaaacaccattgtcac

SEQ ID NO: 15 - DNA, 76 bp, Artificial Sequence
  Oligonucleotide, Lin2 - 19a
cctcagttttgcatagatttgcacaactaaaacaaaaactagaaggaaatagtagtgcaactatgcaaaactgagg

SEQ ID NO: 16 - DNA, 78 bp, Artificial Sequence
  Oligonucleotide, luc1309_wt DNA
accgcctgaagtctctgattaatacatctgtggcttcactattaatcagagacttcaggcgaaaaactagaaggaaaa

SEQ ID NO: 17 - DNA, 78 bp, Artificial Sequence
  Oligonucleotide, luc1309_mut1DNA
accgcctgaagtctctgattaatacatctgtggcttcactattaatcagaaacttcaagcgaaaaactagaaggaaaa

SEQ ID NO: 18 - DNA, 100 bp, Artificial Sequence
  Oligonucleotide
agacggcaacacgtttagatacgttttgactaccaccggacgataaaggaagatcaaaaacaaacgtcaggagacaatcaaaacgtatcaacgtccgtct

SEQ ID NO: 19 - DNA, 86 bp, Artificial Sequence
  Oligonucleotide, 19a (aka 19aENDO)
tagcaggccaccatcagttttgcatagatttgcacaactacattcttcttgtagtgcaactatgcaaaactaacagaggactgcaa

SEQ ID NO: 20 - DNA, 117 bp, Artificial Sequence
  Oligonucleotide, 19aRL aka 19a (Ex) TAR
gaaggaaatagcaggccaccatcagttttgcatagatttgcacaacttccctatgacctcgactacgactggaaatgcaactatgcaaaactaacagaggactgcaaacaaaaacta

SEQ ID NO: 21 - DNA, 94 bp, Artificial Sequence
  Oligonucleotide, 19anbm
gaaggaaatagcagccagttttgcatagttgcaggcagagagctcccaggctctctgcctgcaactatgcaaaactggctgcaaacaaaaacta

SEQ ID NO: 22 - DNA, 117 bp, Artificial Sequence
  Oligonucleotide, RANDC1
atgatctaaaaacggtgctgtgtatgtctgctttgatcaacctctaatagctcgtatgatagtgcagccgctggtgatcactgtgcgaatacgggttgtagcaatgttcgtctgagt

SEQ ID NO: 23 - DNA, 111 bp, Artificial Sequence
  oligonucleotide, 30a
gggtaccctctctcagtaggcagctgcaaacatccgactgaaagcccatctgtggcttcacagcttccagtcgaggatgtttacagtcgctcactgctctggatccctgca

SEQ ID NO: 24 - DNA, 38 bp, Artificial Sequence
  oligonucleotide, Pyr-rich
tttctttacatttctttcttttccgatccttttcggat

SEQ ID NO: 25 - DNA, 27 bp, Artificial Sequence
  Oligonucleotide, 7SK-KO
aacctccaaacaagctctcaaggtcca

SEQ ID NO: 26 - DNA, 27 bp, Artificial Sequence
  Oligonucleotide, 7SK-KOas
tggaccttgagagcttgtttggaggtt

SEQ ID NO: 27 - DNA, 41 bp, Artificial Sequence
  Oligonucleotide, Luciferase-sensel
tcgtcttcgtcccagtaagctatgtctccagaatgtagcca

SEQ ID NO: 28 - RNA, 86 nt, Artificial Sequence
  Oligonucleotide, Predicted but undetected RNA transcript from 19a
uugcaguccucuguuaguuuugcauaguugcacuacaagaagaauguaguugugcaaaucuaugcaaaacugaugguggccugcua

SEQ ID NO: 29 - RNA, 83 nt, Artificial Sequence
  Oligonucleotide, 122 transcript
aucaaacgccauuaucacacuaaauagcuacugcuaggcaauuccuuagcagagcuguggagugugacaaugguguuuguguc

SEQ ID NO: 30 - RNA, 112 nt, Artificial Sequence
  Oligonucleotide, RNA transcript from 19aTAR (19am)
guuugcaguccucuguuaguuuugcauaguugcaggcagaucugagccugggagcucucugccguugugcaaaucuaugcaaaacugaugguggccugcuauuuccuucuag

SEQ ID NO: 31 - RNA, 124 nt, Artificial Sequence
  Oligonucleotide, 122m (aka 122TAR) predicted transcript
uuuuuguuuccuuagcagagcuguggagugugacaaugguguuugugggcagaucugagccugggagcucucugcccuaucaaacgccauuaucacacuaaauagcuacugcuaggcuauuucc

SEQ ID NO: 32 - RNA, 112 nt, Artificial Sequence; identical to SEQ ID NO: 30
  Oligonucleotide, RNA transcript from coligo 19aTAR

SEQ ID NO: 33 - RNA, 109 nt, Artificial Sequence
  Oligonucleotide, RNA transcript from coligo 19aTAR
guuugcaguccucuguuaguuuugcauaguugcaggcagaucugagccugggagcucucugccguugugcaaaucuaugcaaaacugaugguggccugcuauuuccuuc

SEQ ID NO: 34 - RNA, 113 nt, Artificial Sequence
  Oligonucleotide, RNA transcript from coligo 19aTAR
guuugcaguccucuguuaguuuugcauaguugcaggcagaucugagccugggagcucucugccguugugcaaaucuaugcaaaacugaugguggccugcuauuuccuucuagu

SEQ ID NO: 35 - RNA, 114 nt, Artificial Sequence
  Oligonucleotide, RNA transcript from coligo 19aTAR
guuugcaguccucuguuaguuuugcauaguugcaggcagaucugagccugggagcucucugccguugugcaaaucuaugcaaaacugaugguggccugcuauuuccuucuaguu

SEQ ID NO: 36 - RNA, 115 nt, Artificial Sequence
  Oligonucleotide, RNA transcript from coligo 19aTAR
guuugcaguccucuguuaguuuugcauaguugcaggcagaucugagccugggagcucucugccguugugcaaaucuaugcaaaacugaugguggccugcuauuuccuucuaguuu

SEQ ID NO: 37 - RNA, 121 nt, Artificial Sequence
  Oligonucleotide, RNA transcript from coligo 19aTAR
guuugcaguccucuguuaguuuugcauaguugcaggcagaucugagccugggagcucucugccguugugcaaaucuaugcaaaacugaugguggccugcuauuuccuucuaguuuuuguuu

SEQ ID NO: 38 - RNA, 83 nt, Artificial Sequence; identical to SEQ ID NO: 29
  Oligonucleotide, RNA transcript from coligo 122 or linear 122L

SEQ ID NO: 39 - RNA, 84 nt, Artificial Sequence
  Oligonucleotide, RNA transcript from coligo 122
aucaaacgccauuaucacacuaaauagcuacugcuaggcaauuccuuagcagagcuguggagugugacaaugguguuugugucu

SEQ ID NO: 40 - RNA, 82 nt, Artificial Sequence
  Oligonucleotide, RNA transcript from coligo 122
aucaaacgccauuaucacacuaaauagcuacugcuaggcaauuccuuagcagagcuguggagugugacaaugguguuugugu

SEQ ID NO: 41 - RNA, 65 nt, Artificial Sequence
  Oligonnucleotide, Predicted transcript of 122s
aucaaacgccauuaucacacuaaauagggcaauucccuguggagugugacaaugguguuuguguc

SEQ ID NO: 42 - RNA, 61 nt, Artificial Sequence
  Oligonnucleotide, Predicted but undetected transcript from luc1309_wt
cgccugaagucucugauuaauagugaagccacagauguauuaaucagagacuucaggcggu

SEQ ID NO: 43 - RNA, 61 nt, Artificial Sequence
  Oligonucleotide, luc1309_GU2, predicted transcript from luc1309_mut1DNA
cgcuugaaguuucugauuaauagugaagccacagauguauuaaucagagacuucaggcggu

SEQ ID NO: 44 - RNA, 40 nt, Artificial Sequence
  Oligonucleotide, Predicted run-off transcript from the linear template of Fig. 22
guuugcaguccucuguuaguuuugcauaguugcaggcaga

SEQ ID NO: 45 - RNA, 33 nt, Artificial Sequence
  Oligonucleotide, Predicted transcript from lin2-19a
guuuuaguugugcaaaucuaugcaaaacugagg

SEQ ID NO: 46 - DNA, 63 bp, Artificial Sequence
  Oligonucleotide, 19am-1
gaaggaaatagcaggccaccatcagttttgcatagatttgcacaacggcagagagctcccagg

SEQ ID NO: 47 - DNA, 54 bp, Artificial Sequence
  Oligonucleotide, 19-am2
ctcagatctgcctgcaactatgcaaaactaacagaggactgcaaacaaaaacta

SEQ ID NO: 48 - DNA, 30 bp, Artificial Sequence
  Oligonucleotide, splint 19am
caggcagatctgagcctgggagctctctgc

SEQ ID NO: 49 - DNA, 65 bp, Artificial Sequence
  Oligonucleotide, 122m-1
aggaaatagcctagcagtagctatttagtgtgataatggcgtttgatagggcagagagctcccag

SEQ ID NO: 50 - DNA, 59 bp, Artificial Sequence
  Oligonucleotide, 122m-2
gctcagatctgcccacaaacaccattgtcacactccacagctctgctaaggaaacaaaa

SEQ ID NO: 51 - DNA, 31 bp, Artificial Sequence
  Oligonucleotide, splint 122m
tgggcagatctgagcctgggagctctctgcc

SEQ ID NO: 52 - DNA, 27 bp, Artificial Sequence
  Oligonucleotide, Oligo probe
tggaccttgagagcttgtttggaggtt

SEQ ID NO: 53 - DNA, 24 bp, Artificial Sequence
  Oligonucleotide, Oligo probe
agctcctattccatctccctgctc

SEQ ID NO: 54 - DNA, 41 bp, Artificial Sequence; identical to SEQ ID NO: 27
  Oligonucleotide, Oligo probe

SEQ ID NO: 55 - DNA, 30 bp, Artificial Sequence
  Oligonucleotide, Oligo
cggcagagagctcccaggctcagatctgcc

SEQ ID NO: 56 - DNA, 17 bp, Artificial Sequence
  Oligonucleotide, Universal miRNA Cloning Linker (NEB)
ctgtaggcaccatcaat

SEQ ID NO: 57 - RNA, 45 nt, Artificial Sequence
  Oligonucleotide, 5′adaptor (Ambion RLM-RACE kit)
gcugauggcgaugaaugaacacugcguuugcuggcuuugaugaaa

SEQ ID NO: 58 - DNA, 32 bp, Artificial Sequence
  Oligonucleotide, MiRclon3NotI (IDT)
agcggccgcctgcagattgatggtgcctacag

SEQ ID NO: 59 - DNA, 24 bp, Artificial Sequence
  Oligonucleotide, 5′ outer primer (Ambion RLM-RACE kit)
gctgatggcgatgaatgaacactg

SEQ ID NO: 60 - DNA, 35 bp, Artificial Sequence
  Oligonucleotide, 5′ inner primer (Ambion RLM-RACE kit)
cgcggatccgaacactgcgtttgctggctttgatg

SEQ ID NO: 61 - DNA, 31 bp, Artificial Sequence
  Oligonucleotide, 5end122-5 (IDT)
cagcggccgcgaattctatttagtgtgataa

SEQ ID NO: 62 - DNA, 31 bp, Artificial Sequence
  Oligonucleotide, 5end122-6
aagcggccgcgaattcttgcctagcagtagc

SEQ ID NO: 63 - DNA, 30 bp, Artificial Sequence
  Oligonucleotide, 5end122-2 (IDT)
gtgcggccgcgaattcattgtcacactcca

SEQ ID NO: 64 - DNA, 28 bp, Artificial Sequence
  Oligonucleotide, 19aT-fwd (IDT)
gcatggatccgaaggaaatagcaggcca

SEQ ID NO: 65 - DNA, 45 bp, Artificial Sequence
  Oligonucleotide, 3′ RACE adapter
gcgagcacagaattaatacgactcactataggttttttttttttn

SEQ ID NO: 66 - DNA, 32 bp, Artificial Sequence
  Oligonucleotide, 3′ RACE Inner Primer
cgcggatccgaattaatacgactcactatagg

SEQ ID NO: 67 - DNA, 30 bp, Artificial Sequence
  Oligonucleotide, 122aE3end2
taggatcctcacaaacgccattatcacact

SEQ ID NO: 68 - DNA, 5 bp, Artificial Sequence
  Oligonucleotide, RNAP III termination signal
aaaaa

SEQ ID NO: 69 - DNA, 5 bp, Artificial Sequence
  Oligonucleotide, RNAP III termination signal
aaaca

SEQ ID NO: 70 - DNA, 4 bp, Artificial Sequence
  Oligonucleotide, RNAP III termination signal
aaaa

SEQ ID NO: 71 - DNA, 6 bp, Artificial Sequence
  Oligonucleotide
tataat

SEQ ID NO: 72 - DNA, 7 bp, Artificial Sequence
  Oligonucleotide
ttgacat

SEQ ID NO: 73 - DNA, 6 bp, Artificial Sequence
  Oligonucleotide
tataaa

(19) United States
(12) Patent Application Publication
(10) Pub. No.: US 2013/0245102 A1
(43) Pub. Date: Sep. 19, 2013

(54) NOVEL DNA TEMPLATES FOR SMALL RNA PRODUCTION IN MAMMALIAN CELLS

(71) Applicants: Kevin Ryan, New York, NY (US); Christine I. Seidl, London (GB)

(72) Inventors: Kevin Ryan, New York, NY (US); Christine I. Seidl, London (GB)

(73) Assignee: Research Foundation of the City University of New York, New York, NY (US)

(21) Appl. No.: 13/879,254

(22) PCT Filed: Oct. 11, 2011
(86) PCT No.: PCT/US11/55699

§ 371 Date: May 21, 2013

Related U.S. Application Data
(60) Provisional application No. 61/392,301, filed on Oct. 12, 2010.

(51) Int. Cl. C07H 21/04; A61K 31/7052; C12P 19/30

(52) U.S. Cl. CPC C07H 21/04; C12P 19/30; A61K 31/7052
USPC 514/44 R; 536/23.1; 435/89

(57) ABSTRACT

This disclosure describes unique single stranded DNA templates having a characteristic sequence and secondary structure. The DNA templates disclosed herein are useful for making small RNA molecules through promoterless transcription by a mammalian RNA polymerase, and can serve as an effective vector for producing small RNA molecules of interest in vitro, in situ and in vivo in mammalian cells.

### CROSS-REFERENCE TO RELATED APPLICATIONS

This application claims the benefit of priority of U.S. Provisional Application No. 61/392,301, filed on Oct. 12, 2010, the entire content of which is incorporated herein by reference.

### STATEMENT REGARDING FEDERALLY SPONSORED RESEARCH OR DEVELOPMENT

This invention was made with Government Support under NIH grant # NIH1R21GM073944. The Government has certain rights in this invention.

### FIELD OF THE DISCLOSURE

This disclosure relates to novel DNA molecules useful for making small RNA molecules. The DNA molecules disclosed herein are single stranded and have a characteristic sequence and secondary structure that permit a promoter-independent transcription by a mammalian RNA polymerase. Methods for producing small RNA molecules in vitro, in situ and in vivo in mammalian cells by utilizing such DNA molecules are also disclosed.

### BACKGROUND ART

Small RNAs (sRNA) can have significant effects on gene expression and other biochemical processes. Whether encoded genomically or originating through design or laboratory selection, sRNA from classes such as microRNA (miRNA), small interfering RNA (siRNA), ribozymes, short hairpin RNA (shRNA) and RNA aptamers have demonstrated biological activity in vitro and in cells (LEE et al., Curr Opin Chem Biol 10, 282-9 (2006); LARES et al., Trends Biotechnol 28, 570-9; BARTEL Cell 136, 215-33 (2009); RAO et al., Adv Drug Deliv Rev 61, 746-59 (2009)). Biologically active RNA from these and related sRNA classes hold promise for translation to clinical applications if a general means to deliver them safely to human tissues can be found (KIM et al., Nat Rev Genet. 8, 173-84 (2007)).

The microRNAs (miRNA) are naturally occurring RNAs encoded by the genome. They typically act to reduce expression of groups of genes at the post-transcriptional, i.e. the messenger RNA (mRNA) level. The miRNAs work within the context of RNA-protein complexes (miRNPs for miRNA ribonucleoprotein) that include at least one of the Argonaute proteins. An example of an miRNP is the RNA-induced silencing complex (RISC). Part of the miRNA sequence gives the RISC its specificity for targeting the RNA from a transcribed gene, by simple Watson-Crick base-pairing.

Small interfering RNAs (siRNA) represent a class of RNA formed as an intermediate in the process of RNAi (or RNA interference) and can enter the later stages of the miRNA maturation pathway to reprogram the miRNPs and target them to knock-down specific genes through Argonaute 2 (Ago2) mediated slicing activity. Knocking down an mRNA means destroying it, reducing the efficiency of its translation into protein and/or shortening its half-life. siRNAs are used extensively in research to investigate the function of genes by reducing the amount of its mRNA and hence its gene product, the protein, and looking for the result (phenotype) of less protein.

siRNAs are also being actively investigated by many industrial and academic research labs in hopes that they will in some form be able to be used therapeutically. The sequence of an undesirable gene can be used to design siRNAs to target the gene's mRNA via the natural miRNA pathway. In principle, an siRNA against any gene can be designed based on such gene's specific sequence, whereas finding a small molecule drug targeting the gene product is much more difficult and is not always successful.

One of the main problems preventing the use of therapeutic RNAi is getting the RNA into human cells in a human body. Most sRNA intracellular delivery approaches fall within two general categories. One is the direct chemical synthesis of the sRNA (ELBASHIR et al., Nature 411, 494-8 (2001)) with modifications (JEONG et al., Bioconjug Chem 20, 5-14 (2009); WILSON et al., Curr Opin Chem Biol 10, 607-14 (2006)) and nanoparticle (LARES et al., Trends Biotechnol 28, 570-9; Davis et al., Nature 464, 1067-70) or liposomal (SCHROEDER et al., J Intern Med 267, 9-21) packaging to enhance serum stability, tissue targeting and cellular uptake. The chemically or otherwise synthesized RNA double helix is forced into cells using a lipophilic transfection reagent or high voltage. The RNA enters the cells and finds the Argonaute proteins to program a new miRNP, or an siRNP. However, this approach has been shown to work in tissue culture cells only, not in an in vivo system. The other is based on gene therapy vectors that carry the genetic information to make the sRNA, or a pre-processed form of it (COUTO et al., Curr Opin Pharmacol 10, 534-42; MANJUNATH et al., Adv Drug Deliv Rev 61, 732-45 (2009); GLOVER et al., Nat Rev Genet. 6, 299-310 (2005)). Gene therapy vectors are attractive because the otherwise labile and comparatively difficult to synthesize RNA sequence information is held in the more stable form of DNA, and comes packaged in its own delivery vehicle. However, gene therapy carries many risks, including severe immune reactions (WILSON, Mol Genet Metab 96, 151-7 (2009)) and random integration of the DNA into chromosomes, which can lead to cancer (WOODS et al., Nature 440, 1123 (2006)). In addition, like other biologics, gene therapy vectors cannot be characterized to the same extent as synthetic compounds, and can therefore bring with them unnoticed bio-contamination. They also suffer from poor nucleotide (nt) economy, increasing the chances of off-target effects. Given the cost and difficulties of using RNA directly, and the risks associated with gene therapy vectors, alternate approaches for producing sRNA in, or delivering it to, human cells are needed.

Chemical DNA synthesis is simpler and more economical than RNA synthesis (SOMOZA, Chem Soc Rev 37, 2668-75 (2008)). DNA chains over 100 nt are now routinely made, and up to 200 nt are possible (ALLEN et al., Integrated DNA Technologies, Technical Report www.idtdna.com (2007)). Thus, the minimal sequence information needed to encode sRNAs approaching 200 nt are now routinely synthesized.

It is unclear whether the cell's own RNA polymerases (RNAPs) can be harnessed to transcribe synthetic single-stranded (ss) DNA into sRNA. One apparent difficulty that must be overcome was that in order to initiate transcription at specific sites, RNAPs are widely understood to have a general requirement for double-stranded (ds) DNA promoter sequences. It is known, however, that most RNAPs have the ability to initiate “non-specifically” on ss DNA regions, a property that was often exploited to study RNAPs before promoters were discovered (ROEDER, RNA Polymerase (eds. Losick, R. & Chamberlin, M.) 285ff (Cold Spring Harbor Laboratory, 1976)). The structural features of the substrate required for this action of RNAPs, however, are unclear.

### SUMMARY OF THE DISCLOSURE

This disclosure describes a new technology leading to the production of natural or artificially designed small RNAs in mammalian cells. The technology provides an alternative to introducing double-stranded DNA expression vectors, or RNA, into cells. According to this technology, single-stranded (ss) DNA molecules are designed and created that are characterized by certain primary sequence elements in combination with a stem-loop type secondary structure. Such DNA molecules can direct promoter-independent transcription by a mammalian RNA polymerase (such as Pol III) to produce any desired RNA molecule. Thus, the methodology disclosed herein permits the utilization of the endogenous RNA polymerase within mammalian cells, in both the cytoplasm and the nucleus, to produce any desired RNA from the single-stranded DNA templates introduced into the cells. The RNA transcript thus produced can be itself functional or designed to enter an endogenous RNA processing pathway to generate the ultimate functional RNA. Therefore, the methodology disclosed herein allows for an intracellular production of desirable small RNAs, including microRNA (miRNA), miRNA mimics, small interfering RNA (siRNA), or antagonizing antisense RNAs to any of these. Small RNA molecules can be used in various biological and therapeutic applications in the form of, for example, siRNA, miRNA, aptamers, ribozymes, or antagomirs (antisense against miRNA).

### BRIEF DESCRIPTION OF THE DRAWINGS

FIG. 1. RNA Polymerase III Circular Synthetic DNA Template Design Considerations. (a) RNA stem loop-encoding DNA circles. Design begins with the desired RNA, which specifies the complementary DNA (cDNA) portion of the circle. This example depicts a pre-miRNA stem loop, natural examples of which typically contain unpaired bulge nucleotides in the stem. At the base of the stem in the DNA is added a purine-rich loop (e.g., A-rich loop depicted) and a dT or dC at or close to the 3′ end of the stem (boxed) to increase specificity of Pol III initiation, which prefers to start at dC or dT positions. As part of the purine-rich loop sequence may be added a Pol III termination sequence (e.g., d(A)₅ (SEQ ID NO: 68), boxed), to increase the specificity of termination. (b) Similar to (a) except the stem loop here is a pre-shRNA, which contains no bulged nucleotides. Bulge loops in the DNA stem improve transcription, as detailed in FIG. 21. (c) Similar to (a) and (b) except a second Pol III termination sequence, e.g. d(A)₅ (SEQ ID NO: 68), is placed in the purine-rich loop, 3′ to the preferred start site, in order to terminate other loop-initiated transcripts and increase the homogeneity of transcription starting at the desired dC or dT nucleotide.

FIG. 2. Intracellular Delivery Strategies for Delivering Stem-loop RNA-encoding DNA Circles into Mammalian Cells. (a) Cell-penetrating aptamers. Non-genomic DNA aptamers have been identified whose secondary structure binds to cell surface proteins thereby leading to cell internalization. These can be incorporated into the DNA circle sequence as an independent functioning module. (b) Catenation. The topology of DNA circles can be exploited by interlocking another DNA circle which is covalently attached to a molecule conferring cell-penetrating properties, or an antibody leading to accumulation on cells expressing the antigen.

FIG. 3. Rolling Circle Transcription (RCT) approach to expressing miRNA, siRNA and other small RNA. (a) Circular oligodeoxynucleotides (COLIGOs) were made to encode minimal pri-miRNAs. RCT would produce tandemly arrayed primary miRNA stem-loops resembling naturally occurring miRNA clusters to promote entry into the endogenous miRNA maturation pathway, and Argonaute effector complexes (RISC) programming. (b) RNA transcript sequences and the predicted secondary structures of the four COLIGOs used in this study. Mature miRNA are shaded, number in parentheses refers to number of nucleotides in COLIGO and its monomeric transcript, which is shown arbitrarily beginning outside of the stem-loop. This approach was suggested by prior art, but RCT fails to take place in human cells. The current invention solves the problem created by the failure of this approach.

FIG. 4. Circularization of DNA templates for Rolling Circle Transcription. (a) Synthetic 5′ phosphorylated linear DNA sequences were circularized using the thermostable TS2126 RNA ligase. (b) Denaturing polyacrylamide gel electrophoresis (DPAGE) at four stages during miR19 am DNA circle synthesis. Lane 1, crude DNA IDT Ultramer synthesis of template 19 am. Lane 2, after preparative DPAGE. Lane 3, crude circularization product. Lane 4, DNA circle template following Exonuclease I clean-up. Visualization using Stains-All. (c) Verification of circular topology. Nicking of circular templates by 51 nuclease leads first to linear forms, which are then further digested to successively smaller linear forms.

FIG. 5. Transcription of circular oligonucleotides (coligos) by RNA polymerases (a) Schematic illustration showing the sequence and predicted secondary structure of a coligo (left), and its RNA (right). Coligo 122 was taken directly from the template strand of the human gene encoding miR-122. The subscript n describes processivity. Rolling circle transcription (RCT) produces n˜1; while n˜1 indicates single round transcription. Small arrow, circularization site. Shaded region, mature miR-122 or its cDNA. Upper case, DNA. Lower case, RNA. (b) In vitro transcription of coligo 122 by RNAPs of varying evolutionary age and complexity. E. coli RNAP, yeast RNAP II and human RNAPs from HEK293T whole cell extract (WCE) were used for in vitro transcription (IVT). RNA was visualized on a denaturing polyacrylamide (DPAGE) gel by [α-³²P]-UTP incorporation. L, linear. C, coligo. Lanes 1, 4, 7: no template. M, RNA marker. nt, nucleotides. Relative processivity and Relative exposure (i.e. Phosphorimager grayscale setting) are indicated. (c) Sequences and predicted secondary structures of coligos 122TAR, 19a and 19aTAR. (d) IVT using HEK293T WCE followed by DPAGE analysis of uniformly labeled transcripts. Dimer transcripts read-through the termination site one time to produce tandem dimer transcripts. (e) Sequence analysis of transcripts made by human WCE from coligo template 19aTAR. The 19aTAR IVT products were isolated and their cDNA sequenced (see Methods). Tss, transcription start site. (f) Sequence analysis of transcripts made by human WCE from coligo template 122. The 122 IVT products were isolated and sequence using 5′ and 3′ RLM-RACE protocols (see Methods). (g) Coligo 122 was shortened by removing a segment in the stem outside of the miRNA cDNA in order to (1) determine whether transcription depends on the lopp independent of the exact sequence of the 122 stem and (2) produce a coligo whose transcript will be smaller and therefore more closely resemble native pre-miRNA, and hence be a better Dicer substrate.

FIG. 6. RNA stem-loop encoding coligos are general templates for human RNAP transcription, and produce Dicer-cleavable single round transcripts. (a) DPAGE separation of IVT of 122 and shortened form, 122s (see FIG. 1g) in HEK293T WCE from untransfected cells (rDcr−) or cells expressing recombinant human Dicer (Dcr+). L, linear. C, coligo. (b) Four reduced-size coligos modeled on 122s and encoding human miR-15a, miR-21, miR-143 and miR-221, designed to produce stem-loop transcripts close to natural pre-miRNA size. (c) DPAGE separation of IVT of the four reduced size coligos, in HEK293T WCE from untransfected cells (rDcr−) or from cells transiently transfected with recombinant human Dicer (rDcr+). Area of gel containing expected single round transcripts is boxed. Based on analogy with coligos 122 and 19aTAR, the approximate size of the single round transcripts is predicted to be (˜coligo size subtract 7 nt): 15a, 47 nt; 21, 50 nt; 143, 51 nt; 221, 60. L, linear. C, coligo. DNA concentration, 100 nM. (d) RANDC1, unstructured coligo, having the same size (117 nt) and the same G+C (46%) and A+T content as coligo 19aTARR, but was chosen for having as little secondary structure as possible. It was not transcribed in HEK293T cell extract.

FIG. 7. Coligo topology is necessary but not sufficient to template the synthesis of stable, released small RNA transcripts in human whole cell extract. (a) Circularization stabilizes oligonucleotides in human whole cell extract. Circular (C) or linear (L) templates (Input) were recovered (Post) from HEK293T WCE IVT, digested with RNase cocktail to remove cellular RNA, and stained after DPAGE. Linear forms were degraded during IVT; coligos were stable. (b) Sequence and predicted secondary structure of coligo 19aRL. A random loop replaced the TAR loop (cf. FIG. 5c). (c) DPAGE separation of HEK293T WCE IVT of the three coligos and linear forms shown in (a). (d) Transcripts are released from coligo template during IVT. RNase H(RH) was added to (+) or withheld from the indicated IVT reactions at the end of the typical 90 min. incubation period. Products were separated by DPAGE. Lanes 1 and 2, validation of RNase H activity on a ³²P-RNA:DNA hybrid. Reaction in lane 2 was supplemented with total cellular RNA to normalize competing RNAs among all RNase H reactions.

FIG. 8. Coligo concentration dependence and RNA transcript quantitation for HEK293T whole cell extract in vitro transcription (IVT). (a) Quantitative Northern blotting of coligo 19aTAR IVT transcripts. The indicated range of coligo concentrations was used in unlabeled HEK293T whole cell extract IVT reactions and the transcripts compared to known amounts of RNA containing the same Northern probe (a body labeled cRNA) recognition sequence. Numbers below blot indicate transcript molarity estimated after 90 min IVT. Endogenous 7SK RNA was probed separately as a loading control. Asterisk indicates the single round transcript whose concentration was estimated. (b) Quantitative Northern blotting of coligo 122 IVT transcripts, as in (a). (c) DPAGE separation of time course for 100 nM coligo 122 IVT, visualized by [α-³²P]-UTP incorporation. Rel. RNA: relative amount of RNA compared to standard 90 min. IVT. (d) A quantitative LNA Northern blot comparison of endogenous HEK293T miR-19a with the 90 min IVT transcripts made from coligo 19aTAR (100 nM), in the same extract. The ratio of the 19aTAR in vitro single-round transcript (−120 nt) to endogenous miR-19a (23 nt) is 65. Full LNA Northern signal of the DNA input templates can be seen in lanes 5 and 6. In lanes 3 and 4, the DNA templates were added to HEK293T extract and immediately processed without IVT incubation period to show that DNase I treatment of templates ensures they have no visible Northern signal.

FIG. 9. Coligo transcription in human cells. (a) Fate of transfected coligo and linear templates. ³²P-labeled DNA tracers were spiked into unlabeled templates and transfected at 40 nM into HEK293T cells using PolyFect transfection reagent. Templates recovered from harvested cells (in) and media supernatant (out) were adjusted to represent equal percentages of total fractions, and separated by DPAGE. C, coligo. L, linear. M, RNA marker. (b) Coligos are transcribed in human cells. Templates (C or L) were transfected into HEK293T cells for 24 hrs after which time total RNA was assayed by Northern blotting using a 5′ labeled template-specific LNA. The ratio of the 19aTAR single round transcript (−410 nt) to endogenous miR-19a (23 nt) is 26. Endogenous 7SK RNA was probed separately as a loading control. (c) RNase Protection Assay, RPA, on total RNA isolated from cells transfected with coligo or linear 19aTAR. p−, probe; p+, probe alone digested with RNase cocktail. (d) Coligo transcripts accumulate during transfection period. Total RNA of cells transfected with coligo 19aTAR were harvested after the indicated transfection times and probed by Northern blotting using a uniformly labeled in vitro RNA transcript complementary to one complete coligo transcript sequence. 7SK, loading control. (e) Verification of -amanitin activity using a transiently transfected RNAP II promoter driven construct. The Ambion pMir-Report plasmid containing a luciferase reporter gene driven by the RNAP II CMV promoter was transfected into HEK293T cells according to the manufacturer's instructions. Total RNA was isolated, resolved on a denaturing 1.2% agarose gel and probed with a luciferase mRNA-specific probe. The blot was re-probed for 7SK and also stained for rRNA as loading controls. Amanitin values shown are in g/ml. The RNAP II IC50 is 0.025 g/ml-amanitin. The RNAP III IC50 is 20 g/ml-amanitin. RNAP I is not detectably inhibited by 400 g/ml-amanitin32.

FIG. 10. RNAP III is responsible for coligo transcription, which appears to take place mainly in the cytosol of transfected cells. (a) α-Amanitin inhibits coligo transcription at concentrations consistent with RNAP III transcription. HEK293T WCE IVT was carried out with increasing concentrations of α-amanitin. C, coligo 19aTAR, 100 nM. Lanes 3-5: 0.12, 1.2, 120 μg/ml α-amanitin. Rel. SRT: relative amount of single round transcript. (b) Northern blot of total RNA from HEK293T cells transfected with 40 nM coligo 19aTAR with concurrent α-amanitin treatment (Lanes 3-5: 0.12, 1.2, 40 μg/ml). (c) Northern blot of total RNA from HEK293T cells transfected with 40 nM coligo 19aTAR with concurrent RNAP III-specific inhibitor ML-60218 treatment at 68 μM. DMSO, inhibitor solvent. U2 snRNA probed as loading control. (d) DPAGE of ³²P tracer-labeled 19aTAR templates recovered from HEK293T transfection after separation of nuclear and cytosolic fractions. Inset: Western blot assessment of fractionation, β-tub., β-tubulin (cytosolic protein). H4, histone H4 (nuclear protein). (e) Northern blot of RNA isolated from HEK293T nuclear and cytosolic fractions 24 hours post transfection with 40 nM coligo 19aTAR. (f) HEK293T nuclear and cytosolic extract IVT in the presence of increasing α-amanitin (a, lanes 3-5 and 10-12: 0.12, 1.2 and 120 μg/ml), DMSO (D), or ML-60218/DMSO solution (ML). Inset: Western blot assessment of fractionation, β-tub., β-tubulin (cytosolic protein). CstF-77, (nuclear protein).

FIGS. 11-20 depict the secondary structures of various coligos.

FIG. 21 depicts siRNAs containing identical mature siRNA guide sequence but differences in passenger strand as a consequence of facilitating transcription.

FIG. 22 depicts a linear template used to generate small RNA by run-off transcription in human cells or in vitro. A-rich loop is an example of a more general purine-rich loop with dC or dT embedded near stem to specify initiation site.

FIG. 23 depicts a second example (lin2-19a) used to generate a small run-off RNA transcript.

FIG. 24 depicts a linear precursor of a coligo which can be circularized in situ by cellular ligase, or kinase and ligase acting sequentially.

### DETAILED DESCRIPTION

The present inventors have identified for the first time that synthetic single-stranded DNA molecules, designed based on sequence and secondary structure principles disclosed herein, can trigger promoter-independent transcription by a mammalian RNA polymerase III to produce desired RNA molecules. As disclosed herein, single-stranded DNA templates having a characteristic stem-loop structure, in both circular and linear forms, can direct a mammalian RNA polymerase III to initiate transcription at a defined nucleotide, and terminate to produce single copy transcripts of defined lengths without generating multimers. The characteristic stem-loop structure of the present single-stranded DNA template, in combination with the sequence rules disclosed herein, serves in place of a functional RNA polymerase promoter sequence.

The methodology disclosed herein permits the design of single-stranded (ss) DNA molecules based on any small RNA molecules desired to be generated, and delivery of the designed ss DNA templates into mammalian cells to produce the desired small RNA molecules by utilizing the endogenous mammalian RNA polymerase III activity. The DNA templates can encode a mature or functional small RNA, or encode a pre-RNA which enters an endogenous RNA processing pathway to produce a mature or functional small RNA.

The inventors have also discovered that mammalian RNA Polymerase III, present in the nucleus as well as in the cytoplasm where it carries out its role in the innate immune response, initiates promoterless transcription on a single-stranded DNA molecule having a characteristic stem-loop structure. Thus, ss DNA templates need only to enter the cytoplasm to be transcribed, without necessarily entering the nucleus.

While the ss DNA templates disclosed herein direct the generation of single (i.e., monomeric) copies of RNA transcripts by a mammalian RNA polymerase III in contrast to the production of repeating multimer transcripts as a result of a rolling circle transcription (RCT) by a bacterial or bacteriophage RNA polymerase, the levels of RNA transcripts produced from ss DNA templates by a mammalian RNA polymerase III have been shown to be comparable to the levels of endogenous microRNAs.

The ss DNA templates disclosed herein provide a number of advantageous as compared to the existing approaches for generating and delivering RNA molecules. ss DNA molecules are much more stable than synthetic RNA molecules. ss DNA molecules are also easy and inexpensive to make. Instead of using large (5,000 to 10,000 base pairs) double-stranded DNA with a promoter sequence, small single stranded synthetic DNAs are used, which can be 50-130 nucleotides, just enough to encode the smallest RNA that can enter the natural miRNA maturation pathway. Synthetic ss DNA molecules are also free from biological contamination, and are not associated with chromosomal integration. Further, in addition to conventional techniques for delivering nucleic acid molecules into cells, the circular DNA templates described herein provide unique opportunities to be rendered capable of entry into human cells without the use of transfection reagents or electroporation, as further described below.

The various features of the ss DNA molecules of this invention, and methods of using such ss DNA molecules for producing small RNA molecules in mammalian cells, are further described below.

Single-Stranded DNA Template

As used herein, the term “single-stranded DNA template” refers to a single DNA strand that encodes a desired RNA molecule. In contrast to conventional double-stranded DNA templates requiring a promoter sequence for transcription, the single-stranded DNA templates disclosed therein are transcribed by a mammalian RNA polymerase, independent of any requirement for a promoter sequence, to produce a complementary anti-parallel RNA strand. Additionally, unlike the documented rolling circle transcription (RCT) carried out by bacterial and bacteriophage RNA polymerases, a mammalian RNA polymerase III (pol III) transcribes the single-stranded DNA templates disclosed herein into a single RNA transcript in each transcription action, as opposed to generating repeating units (multimers) of RNA transcripts in a RCT.

In specific embodiments, the single-stranded DNA templates disclosed therein are devoid of a promoter sequence, including promoter sequences recognized by either a eukaryotic (such as mammalian) RNA polymerase or a prokaryotic RNA polymerase. Both prokaryotic and eukaryotic promoter sequences are well characterized in the art, and include, for example, a Pribnow box (or the “−10 element”), characterized by the consensus sequence TATAAT (SEQ ID NO: 71) or variants thereof; a −35 element, characterized by the consensus sequence TTGACAT (SEQ ID NO: 72) or variants thereof; and a “TATA box”, characterized by the consensus sequence TATAAA (SEQ ID NO: 73).

As opposed to a functional RNA polymerase promoter sequence, the single-stranded DNA templates of this disclosure have a characteristic secondary structure and sequence elements, which in combination, direct a promoter-independent transcription by a mammalian RNA polymerase III. Typically the single-stranded DNA templates are characterized by a stem-loop structure, at whose junction initiation takes place.

By “stem-loop” it is meant that the single-stranded DNA molecule forms a secondary structure having a “stem-loop” pattern where two regions of the DNA strand base-pair to form a double helix (the “stem”) that ends in an unpaired loop. The formation of a stem-loop structure requires the presence of a sequence that can fold back on itself to form a paired double helix.

Without intending to be bound by any particular theory, it is believed that this stem-loop secondary structure in the template DNA mimics the transcription “bubble” seen in a double-stranded DNA template, and together with a purine-rich sequence present in the loop of the template DNA, directly adjacent to the 3′ end of the stem, and preferably containing a dC or dT within six nucleotides of the stem and surrounded by some of the purines of the purine rich loop, provokes a mammalian RNA polymerase III to initiate transcription at a nucleotide adjacent to the loop-stem junction, preferably the dC or dT of the template DNA. In other words, the characteristic stem-loop structure of a single-stranded DNA template, in combination with a purine-rich sequence present in the loop, serves in place of a functional RNA polymerase promoter sequence.

In some embodiments, the stem-loop structure of the template DNA has, in addition to the loop from where transcription is initiated, a second, smaller loop at the opposite end of the stem region. To distinguish the two loops in a DNA template, the larger loop from which transcription is initiated, is also referred to herein as the “primary” or “large” loop, while the other terminal loop at the opposite end of the stem region is referred to herein as the “small” loop.

Generally speaking, the large or primary loop has about 10-25 nucleotides, does not have significant predicted secondary structure, and is purine-rich, but not pyrimidine (C/T) rich.

For example, the primary loop can consist of at least 10 nucleotides, but not more than 25 nucleotides. In specific embodiments, the primary loop consists of 11-22 nucleotides, namely, 11, 12, 13, 14, 15, 16, 17, 18, 19, 20, 21 or 22 nucleotides.

By “purine-rich” is meant that the number of deoxyadenosines (“dA”) should constitute at least 30% of all the nucleotides in the loop, which can be at least 35%, 40%, 45%, 50%, 55%, 60%, 65% or higher. The sum of the dG and dA should exceed 50% of the large loop nucleotides, and can be 55%, 60%, 65%, 70%, 75%, or higher. In some embodiments, most of the purines in the large loop are dAs; for example, 65%, 70%, 75%, 80% or higher of the purines are dAs.

By “pyrimidine rich” is meant that the combined number of dC and dT constitutes at least 60%. Generally speaking, the primary loop should not be rich in pyrimidines; in other words, the combined number of dC and dT is preferably not more than 60%, 50%, 40% or even 30%.

The above sequence constraints are followed to trigger transcription at a nucleotide proximate to the base of the stem of the template molecule. It is known that RNA polymerases (RNAPs) prefer to start at dC or dT, putting an rG or rA, respectively, at the 5′ end of a transcript. Therefore, in specific embodiments, the primary loop has a dC or dT nucleotide 5′ proximate to the base of the stem of the template, e.g., within 5-6 nucleotides from the base of the stem of the template, namely 1, 2, 3, 4, 5, or 6 nucleotides apart from the base of the stem of the template. Such dC or dT nucleotide is preferably embedded within the purines in a purine-rich sequence, and represents the most frequently used transcription start site herein. In specific embodiments, the large loop includes a sequence of multiple purines (e.g., 4, 5, 6, 7, 8, 9 or more purines) with a dC or dT nucleotide embedded in the purines and positioned at within 5-6 nucleotides 5′ from the base of the stem—such dC or dT will serve as the transcription start site (TSS) for pol III.

When the template DNA includes a second terminal loop, this second loop is smaller than the primary loop and should be at least 3 nucleotides in size, and generally does not have more than 9 nucleotides. In specific embodiments, the smaller loop of the template DNA consists of 3, 4, 5, 6, 7 or 8 nucleotides.

Without intending to be bound by any particular theory, it is believed that having only one large loop in the template promotes intramolecular secondary structure formation in both the template and its transcripts, which would minimize RNAse H activity. One example of a coligo having two large loops (19aRL) has not produced detectable RNA transcripts in vitro, possible because the transcripts produced form persistent RNA:DNA hybrids, which are degraded by RNase H. It is also possible that a second large loop may be occluded by other cellular single-stranded DNA binding proteins, or that two polymerases create a steric conflict. It is noted that the size of the RNA loop in the transcript may be slightly different from the size of the loop in the DNA template, depending on the secondary structure of the RNA hairpin. Design of a DNA template should balance what works well for transcription by a mammalian RNAP III with, where desired, optimal RNA loop stability and preference by subsequent RNA processing enzymes, such as Dicer.

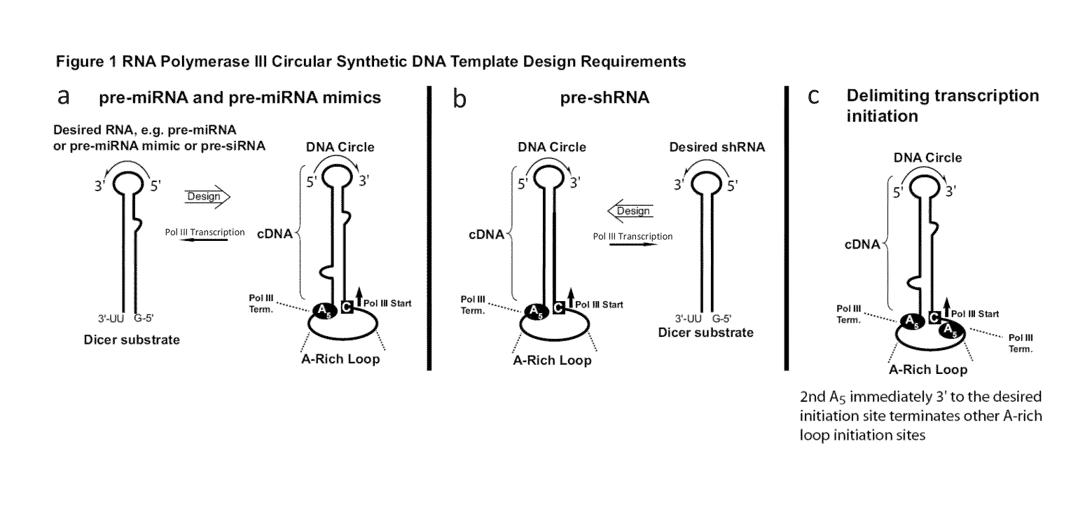

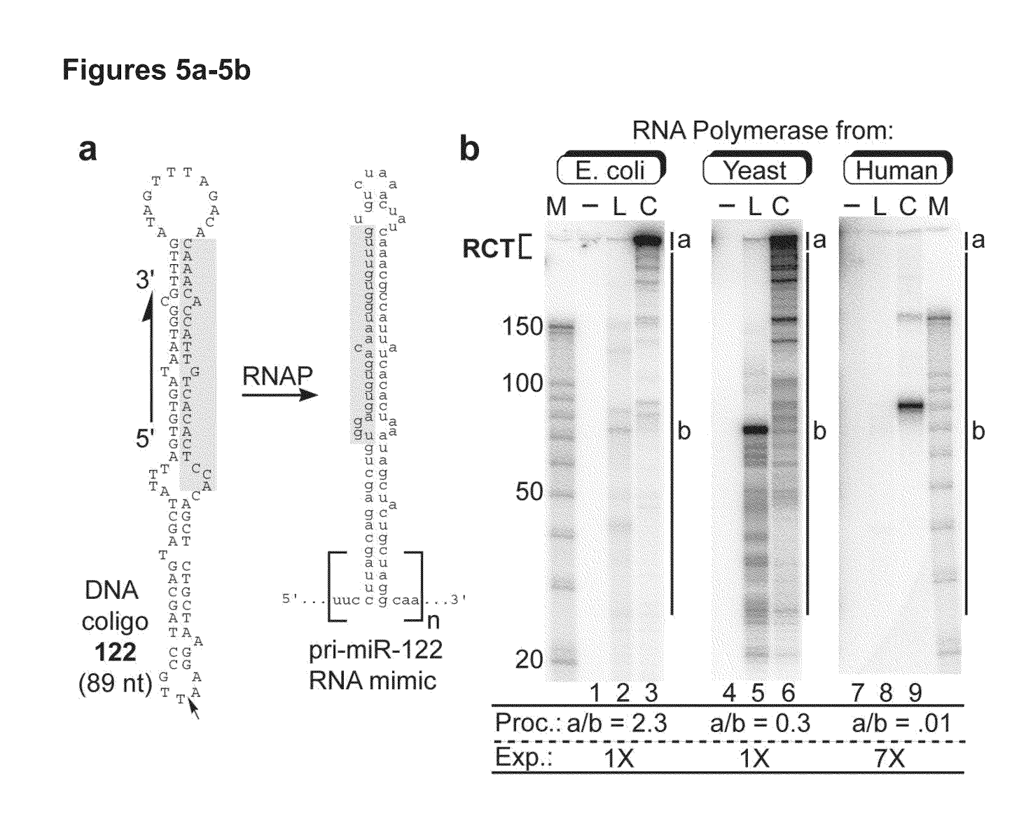

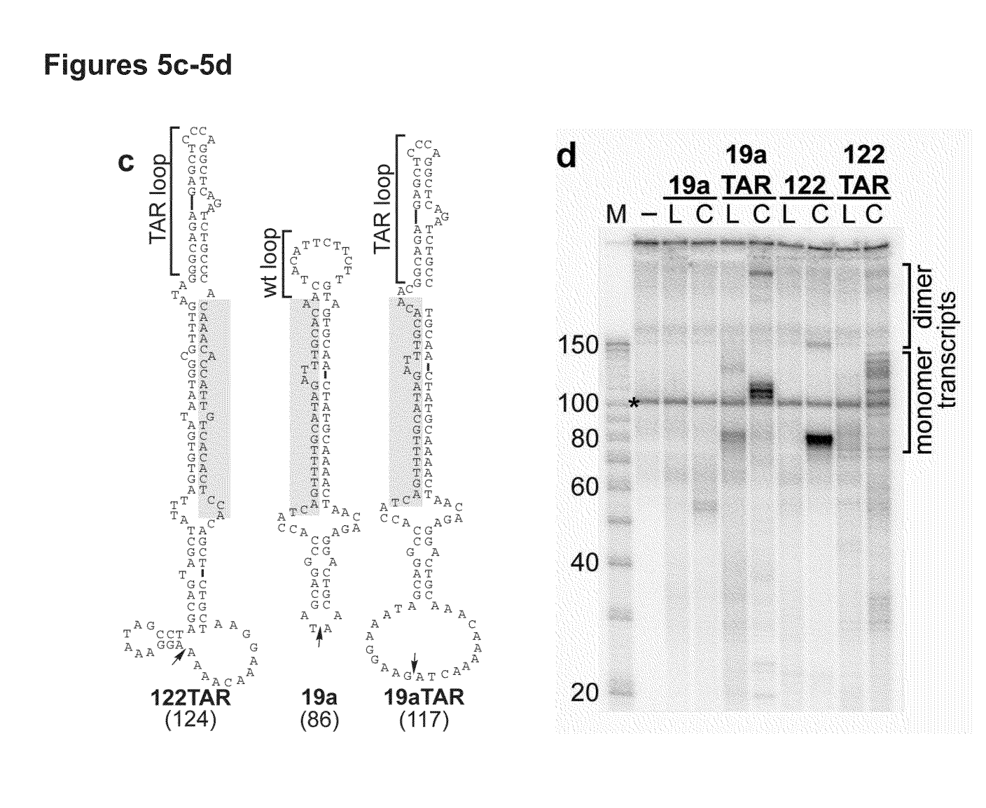

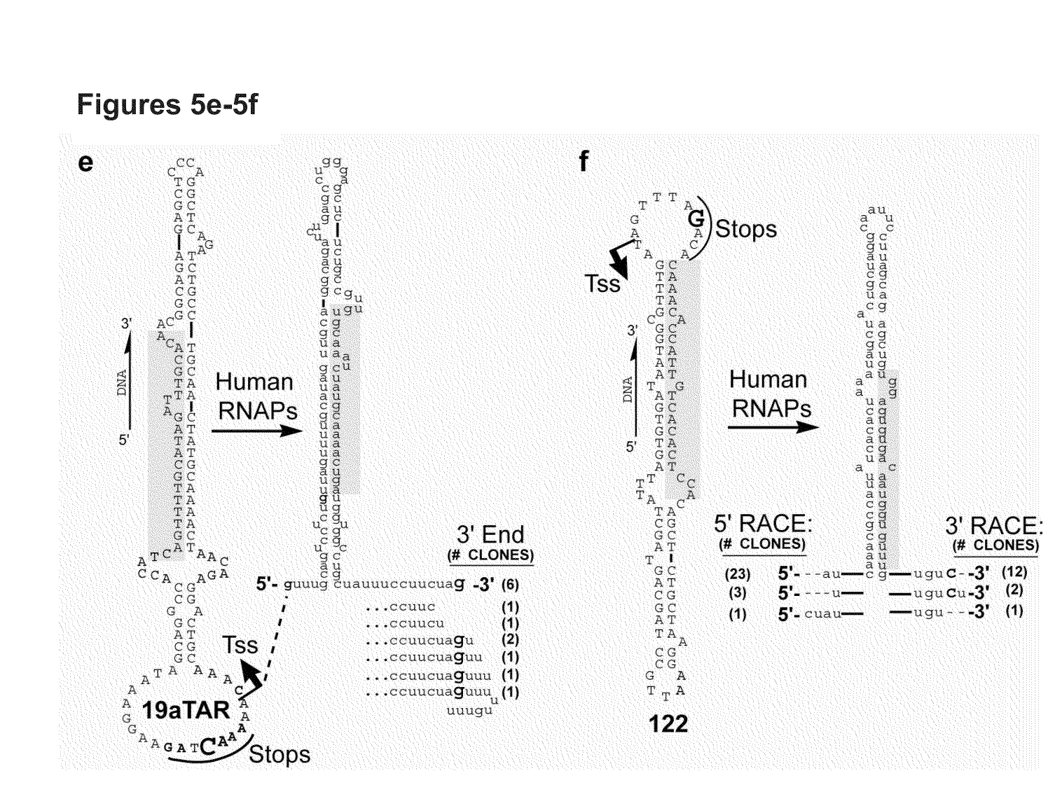

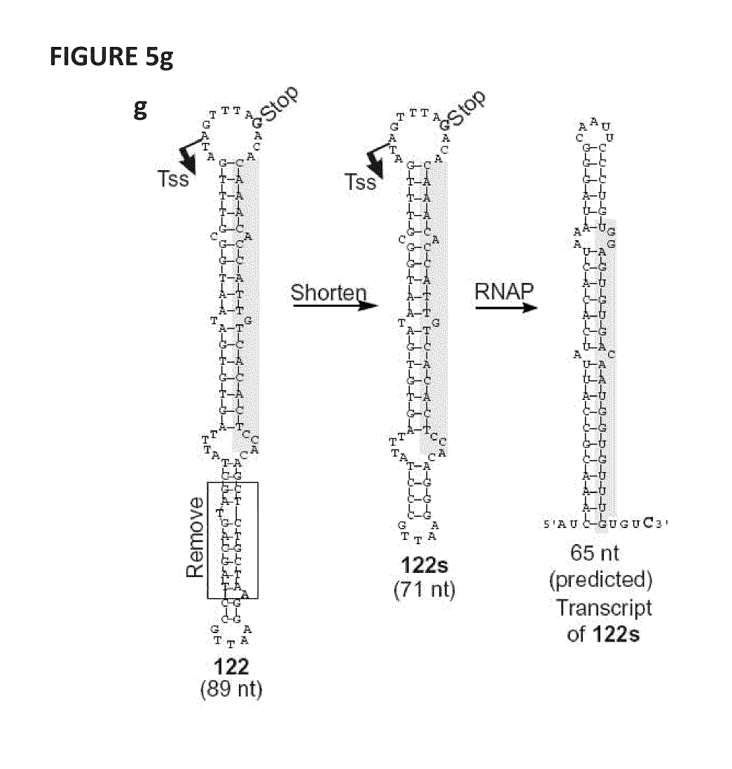

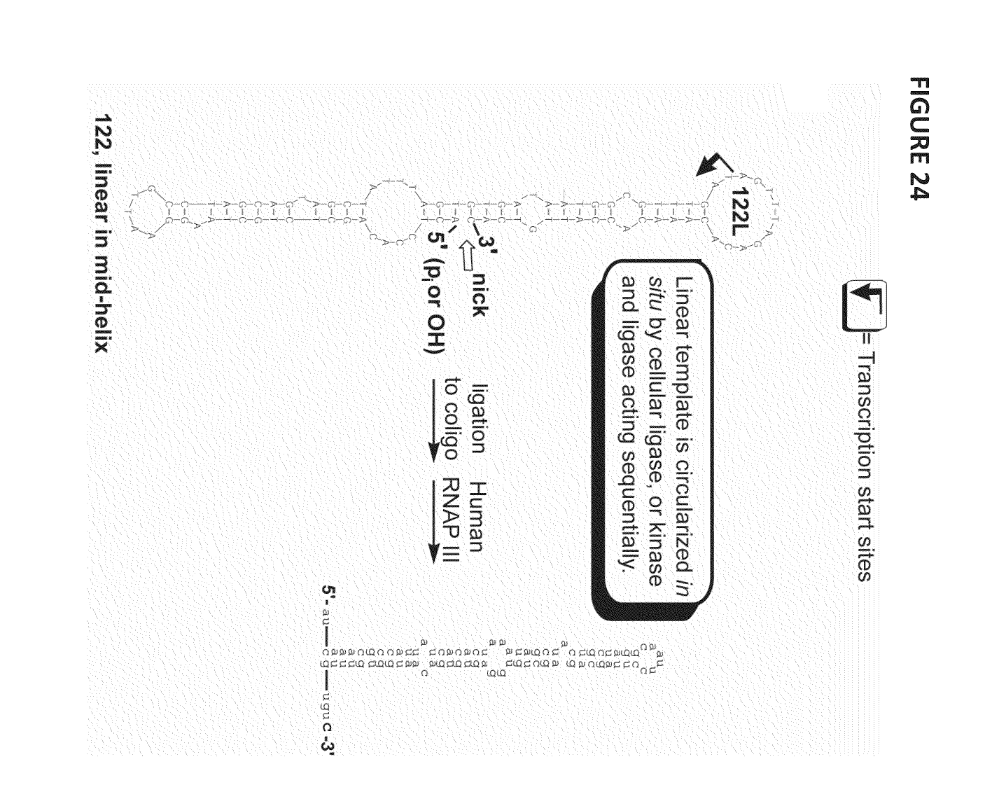

When the template DNA includes a second loop, the template DNA can be a circular molecule (without discontinuity) or a linear molecule (with a point of discontinuity), as illustrated in FIG. 1a and FIG. 24, for example. Given a circular template (also called “coligo”), transcription by a mammalian RNAP III, which is initiated in the primary loop, will progress along the template molecule. Unlike the E. coli and bacteriophage RNAP, however, the mammalian RNAP III does not have the processivity required to conduct RCT and produce repeating units of RNA transcripts. Even in the absence of a termination signal in the template coligo DNA, mammalian RNAP III mostly produces single round transcripts, with only a small amount of dimmers to tetramers (FIG. 5b). While inclusion of a termination signal may not be absolutely necessary, in some embodiments, one or more (e.g., two) RNAP termination sequences are included in a coligo and placed at the junction between the stem and one of the loops or entirely within one of the loops. By “at” a stem loop junction, it is meant that a termination sequence can be partly in the stem and partly in a loop. If a termination sequence is present at the junction between the stem and the 5′ end of the primary loop or entirely within the primary loop, transcription will proceed almost a full round of the circle until the termination sequence is reached, and the RNA transcript produced is of a stem-loop (or “hairpin”) configuration, with the loop in the RNA corresponding to the small loop of the DNA template. If a termination sequence is present at the junction between the stem and either end of the small loop or wholly contained in the smaller loop, transcription will proceed past the stem region until reaching the termination sequence, and will produce a small RNA molecule that does not have significant secondary structure. The presence of a termination sequence entirely within the stem region, however, has not been observed to cause termination of transcription, but should preferably be avoided if the desired RNA molecule requires further transcription.

There is no particular restriction on the RNAP III termination signals which can be used in a template DNA, as long as they serve to terminate transcription by RNAP III. Generally, RNAP III termination signals are A-rich. Specific examples of RNAP III termination signals include AAAAA (SEQ ID NO: 68), AAACA (SEQ ID NO: 69), and AAAA (SEQ ID NO: 70). Additional examples of RNAP III termination signals are described by Orioli et al., Nucleic Acids Res. 39 (11): 5499-5512 (2011), the entire content of which is incorporated herein by reference. It is possible that a dA-rich sequence with an embedded dC or dT, which serves a transcription initiation sequence herein, also acts as a polIII termination signal.

With respect to the stem region of the template DNA, the stem generally is at least 8 nucleotides in length. The stem can however be of any length, as permitted by the art of chemical DNA synthesis, and provided that the RNA transcript deriving from the template has sufficient intramolecular secondary structure to avoid becoming an RNase H substrate. Multiple stems are possible as well, to produce functional tertiary structures as are known for aptamers and ribozymes.

In specific embodiments, the stem is an imperfect stem, i.e., imperfectly base-paired stem. That is, the bases in the stem do not form perfect Watson-Crick base pairs with each other, and therefore the stem contains one or more “bulges” of unpaired bases. It is believed that the form of RNAP III responsible for transcription has fairly low helicase activity. Therefore, the presence of a bulge in the stem facilitates the progression of transcription along the template.

The size of the bulge is at least one unpaired nucleotide or mismatch pair of nucleotides, up to 6-7 consecutive pairs of unpaired bases. Often, the bulge contains 1-4 consecutive pairs of unpaired bases. The location of the bulges in the stem can vary. In some embodiments, at least one bulge is present in the stem close to the junction of the stem and the primary loop. By “close” it is meant the bulge is within 10-15 nucleotides, within 6-8 nucleotides, or even within 3-5 nucleotides (e.g., 2, 3, 4, or 5 nucleotides apart from the base of the stem).

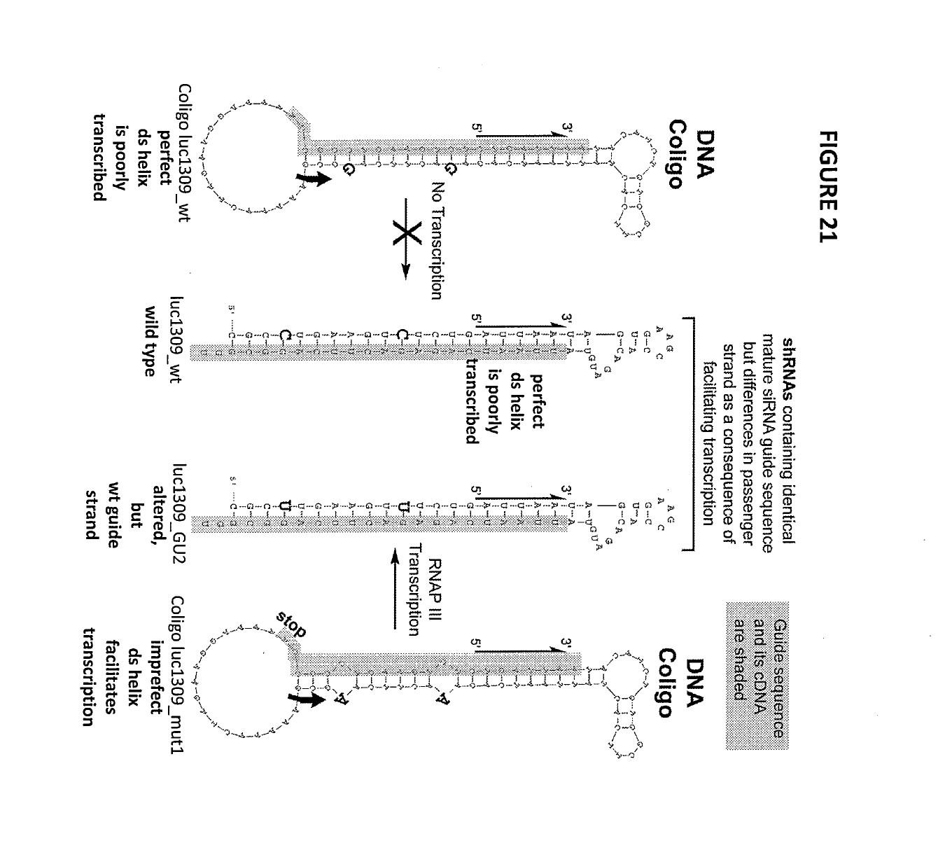

It is convenient to design DNA templates with one or more bulges that encode pre-miRNA, aptamers, and ribozymes, as these RNAs generally have imperfect stems. For shRNAs which typically have perfect stems, DNA templates can also be created to include one or more bulges to achieve effective transcription by RNA polymerase III and still preserve the perfect base pairing of the shRNA stem at the same time. More specifically, in a given desired shRNA, the sequence of the siRNA* arm, i.e. the arm containing the passenger strand, may be altered in such a way as to introduce unpaired nucleotides and bulge loops at the DNA level but not the RNA level. This is done by taking advantage of the greater G-U wobble base pair stability in RNA than the complementary A-C mismatch pair in the DNA. Specifically, within the cDNA of the fully base-paired stem, bulges are created by mutating a G (at selected G-C base pairs) into an A, creating an A-C mismatch in the stem, facilitating transcription by human RNAP III. In the pre-miRNA, what was a C-G base pair becomes a U-G base pair, preserving the perfect base-pairing of the stem. These changes are exemplified in FIG. 21, in which two G-C base pairs are changed to A-C mismatches, making the coligo encoding a known luciferase shRNA (Nature Methods 3 (9): 707, 2006) into a good RNAP III substrate. Therefore, mutations can be made where necessary in the stem of the DNA template, as long as care is taken with respect to the end of the helix, as this portion can influence whether the 5′ or 3′ arm is incorporated into RISC.

While there is no specific restriction on the length of the single-stranded DNA template, in some embodiments, the single stranded DNA template has fewer than 250 nucleotides, while in other embodiments, the single stranded DNA template has fewer than 200, 175, 150, 125, 100, or 75 nucleotides.

The single-stranded DNA template of this invention can be in a circular (i.e., closed without discontinuity) form, or a linear, open form (the 5′ and 3′ ends of the DNA strand are not joined together). Both circular and linear forms of DNA templates can support a promoterless transcription by RNAP III as long as the DNA templates can form a stem-loop secondary structure as discussed above.

For example, single-stranded DNA circles can be designed to form a secondary structure having a stem region and two terminal loops, with the larger loop serving as the primary loop where transcription is initiated. Construction of DNA circles can be achieved using techniques described in the art. For example, linear “ultramer” sized (70-200 nt) oligonucleotides can be synthesized first (IDT, Coralville, Iowa) and are chemically phosphorylated at the 5′ end during synthesis. Subsequently a DNA ligase, optionally with a smaller splint oligonucleotide, is used to join the ends of the linear oligonucleotide into a circular DNA, as described by Shabarova (Biochimie 70: 1323-34, 1988), Kool (J. Amer. Chem. Soc. 113: 6265-6266, 1991), and Diegelman and Kool (Nucleic Acids Res 26: 3235-41, 1998). As another example, a thermostable RNA ligase from bacteriophage TS2126 can be used, which has previously been found to be effective at circularizing ss DNA molecules (Blondal et al., Nucleic Acids Res. 33: 135-142, 2005).

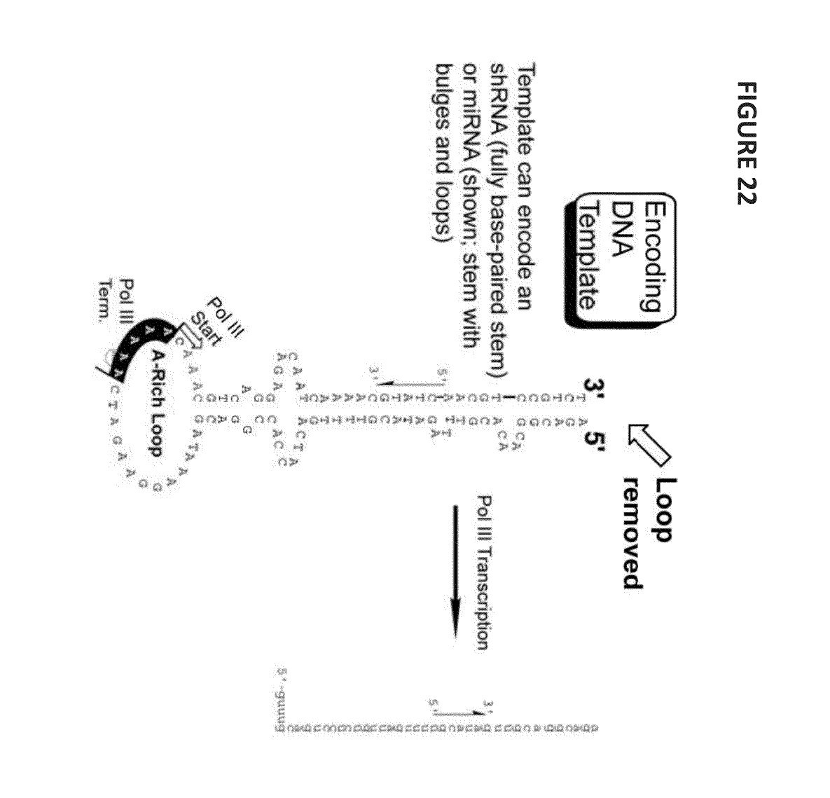

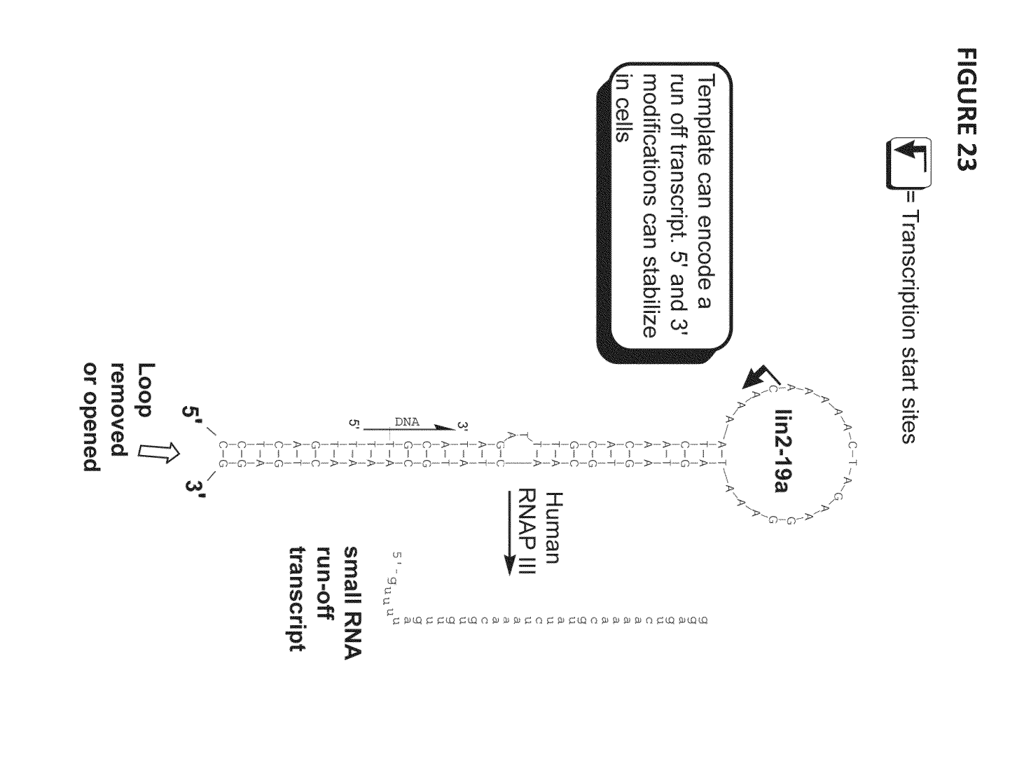

Single-stranded linear DNA templates can also be designed to form a secondary structure, which has a stem region with either one or two terminal loops, depending on the location of discontinuity relative to the base paired stem region. In some embodiments, a single-stranded linear DNA template has its point of discontinuity at the end of the stem region, and the template forms a hairpin (or stem-one loop) secondary structure. See, e.g., FIGS. 22-23. In other embodiments, a single-stranded linear DNA template has its point of discontinuity towards the middle of the stem region, and the template forms a stem-double loop secondary structure. See, e.g., FIG. 24.

Two examples of linear templates are illustrated in FIGS. 23-24, respectively. In FIG. 23, linearizing a coligo remote from the RNAP III initiation loop and transcription start site, does not interfere with the initiation site structure, and allows the coligo to be converted to a “run-off” transcription template, analogous to traditional promoter-containing run-off transcription templates (Milligan et al., Nucleic Acids Res. 15: 8783-98, 1987). The 5′ and 3′ ends of linear templates can be easily chemically modified to protect against degrading exonucleases. In FIG. 24, the synthetic linear precursor of a coligo, with either a 5′ phosphate or no modification, is made such that the point of discontinuity is in a base-paired, double helical segment of the template. Cellular ligase enzyme(s) circularize the template by ligation within human cells and cell extracts. If the template is made without a 5′ phosphate, cellular kinases first phosphorylate the 5′ end in situ, followed by ligation as above.

Circular and linear templates have their respective advantages. Circularization of templates (1) generally increases stability of the templates in cells and cell extracts, (2) stabilizes the secondary structure at the stem-larger loop junction, promoting productive recruitment of RNAP III, and (3) the RNAP III termination signals, in an artificial single-stranded form, work with increased efficiency at stem loop junctions within the coligo. In addition, circular DNA templates are especially useful for making RNA molecules having a hairpin shape, such as pre-miRNAs and shRNAs. On the other hand, linear DNA templates can be especially useful for making RNA molecules, particularly small RNA molecules, which do not have significant secondary structures, such as a fully mature siRNA or miRNA, as they are incorporated into Argonaute protein complexes such as RISC. In addition, linear templates are less expensive to make than circular templates, and can be chemically modified at either or both of its 5′ and 3′ ends to reduce degradation.

Derivatives and Modifications

The primary loop in the template DNA serves to trigger initiation of transcription, as well as termination of transcription in cases of templates with double terminal loops, but is itself not transcribed. Since the DNA is chemically synthesized, this loop can harbor unnatural nucleotide mimics that in rolling circle transcription would interfere with transcription. These unnatural nucleotide mimics may be of any type or covalent modification such that they confer on the DNA circle favorable properties such as serum stability, tissue targeting and intracellular delivery, as long as they do not compromise the Pol III initiation or termination functions of the primary loop. Examples of unnatural nucleotide mimics include those covalently attached to cholesterol or cell-penetrating peptide conjugates for cell permeability, or phosphorothioates, 2′-OMe (methyl) and 2′-F (fluoro) nucleotide modifications for serum and deoxyribonuclease stability.

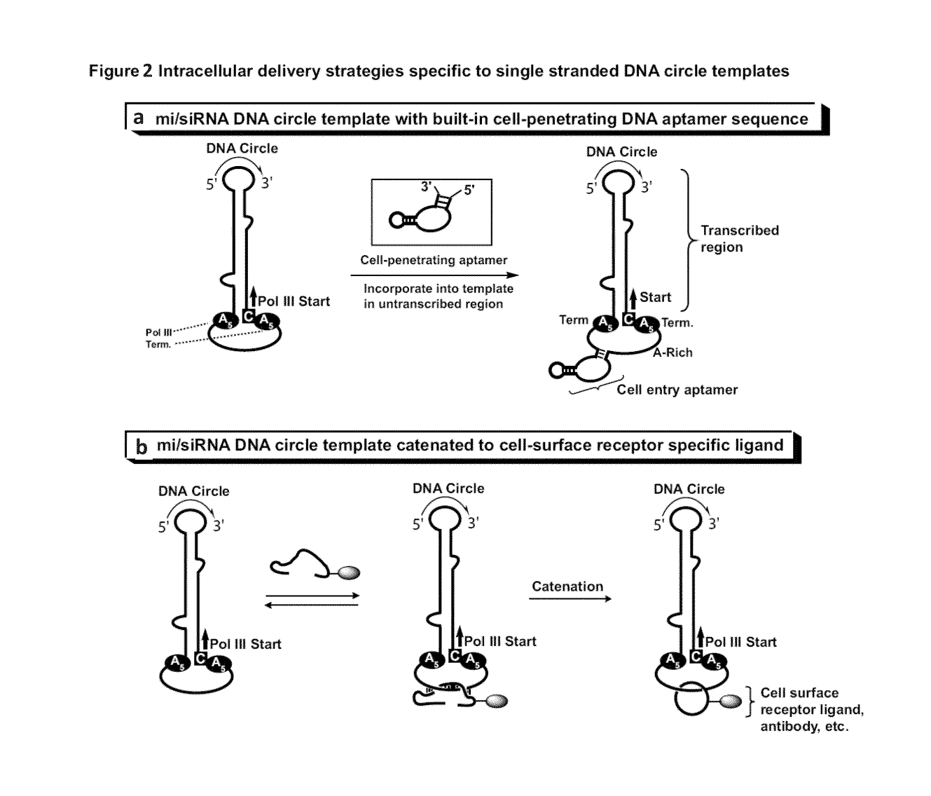

In some embodiments, the primary loop can also be designed to include a DNA aptamer sequence, such as a cell-penetrating aptamer sequence. Cell-penetrating aptamers are DNA sequences that are selected from large random collections of nucleotide sequences for their ability to bind to a cell-surface receptor and be internalized along with the receptor as part of the receptor's natural function (cell-SELEX). See, e.g., Zhou and Rossi (Curr Top Med Chem 9: 1144-57, 2009), and Xiao et al. (Chemistry 14: 1769-75, 2008). For example, during synthesis, a cell-internalizing DNA aptamer such as sgc8 (Shangguan et al., Proc Natl Acad Sci USA 103: 11838-43, 2006; Shangguan et al., Anal Chem 80: 721-8, 2008) may be inserted into the primary loop to direct the DNA template to target and enter lymphoblastic leukemia (ALL) T-cells (Shangguan et al. 2006; Shangguan et al. 2008) (FIG. 2a), where it can undergo Pol III transcription to deliver a therapeutic siRNA specifically to these cells.

In other embodiments, the circular topology of a DNA circle template can be exploited by forming an interlocking catenane with another DNA circle to which is covalently attached a cell-targeting and/or cell-internalizing molecule such as, for example, cholesterol, a cell-penetrating peptide, a cell surface receptor ligand, or an antibody to such a receptor, for example the transferrin receptor, which is overexpressed on tumor cells (Agarwal et al., Int J Pharm 350: 3-13, 2008) (FIG. 2b). This approach has the advantage of linking the template circle through the strength of a covalent bond (since a covalent bond in one of the circles must be broken to separate them) without creating a covalent modification or lesion directly to the DNA circle template, which is known to interfere with transcription (Sebestyen et al., Nat Biotechnol 16: 80-5, 1998). Furthermore, it is known that a DNA circle which is topologically threaded by a DNA transcription template is easily pushed out of the way by an RNA polymerase transcribing the template (Ryan, Ph.D. Thesis, Department of Chemistry: University of Rochester, 1996). Thus the interlocking DNA circle is not expected to impede Pol III transcription of the DNA template circle. Catenation may be achieved by allowing complementary base pairing to form between the DNA template and a linear DNA molecule modified with the cell-targeting or -internalizing modification (represented by the shaded oval in FIG. 2b) which is then closed by a nucleic acid ligase (e.g., Blondal et al. 2005, supra), covalent reactions such as “click” chemistry (El-Sagheer and Brown, Chemical Society Reviews 2009: 1388-1405, 2009) or other established chemical strategies (Liu et al., J Am Chem Soc 130: 10882-3, 2008).

Methods of Making RNAs

The single-stranded DNA templates described herein are useful for encoding and making desired RNAs. In typical embodiments, the single-stranded DNA templates are chemically synthesized. In some embodiments, the synthetic single-stranded DNA templates have fewer than 200-250 nucleotides, and are said to encode “small RNAs”, i.e., RNAs that have fewer than 250 nucleotides, or fewer than 200, 175, 150, 125, 100, 75 or even 50 nucleotides. Useful small RNAs include: minimized primary-microRNA, pre-shRNA, shRNA, pre-microRNA, microRNA (miRNA), small interfering RNA (siRNA), aptamers, antisense RNA, ribozymes, antisense miRNA (i.e. antagomirs), tRNA and small ribosomal RNA, and any other small RNAs.

The RNAs made from the single-stranded DNA templates can be mature functional RNAs, or can be precursors that are able to enter endogenous RNA processing pathways to generate the ultimate mature and functional RNAs. Reported studies on siRNA, shRNA and minimal pri-miRNA have demonstrated that, no matter how an siRNA or miRNA precursor is generated and made to enter cells, it can successfully enter the cellular miRNA biogenesis pathway at the point corresponding to its size and secondary structure. For example, siRNA will enter the RISC directly, though extending its length will turn it into a Dicer substrate, which increases the efficiency of RISC loading. ShRNA made from plasmid or viral vectors in the nucleus enters the nuclear export pathway and then is processed by Dicer and loaded onto RISC in the cytoplasm. Minimized pri-miRNA made from plasmid or viral templates driven by RNAP II promoters enter the natural miRNA processing pathway yet one step earlier, and are initially processed by Drosha/DGCR8, followed by export, Dicer and RISC loading. Thus, it is believed that transcripts made from the DNA templates disclosed herein will enter the miRNA biogenesis pathway at the appropriate step when designed to be a substrate for Dicer, for example.

RNA stem loops made from our vectors can be shRNA (Paddison et al., Genes Dev 16: 948-58, 2002), as illustrated in FIG. 1b and FIG. 21, or a pre-miRNA, as illustrated in FIG. 1a, which, when produced by cytoplasmic RNA Pol III, gives rise to the RNA in the cytoplasm where Dicer endonuclease complexes function in mammalian cells (Billy et al., Proc Natl Acad Sci USA 98: 14428-33, 2001). Producing the RNA in the same cellular compartment where Dicer is found facilitates processing of the transcript into a mature siRNA or miRNA, respectively, and thereby also facilitates its subsequent loading into the RNA Induced Silencing Complex (RISC) (Kim et al., Nat Biotechnol 23: 222-6, 2005) which also functions in the cytoplasm. The RNA stem loop can be a pre-shRNA or mimic a primary-miRNA (pri-miRNA), having extra flanking sequences coded for in the DNA circle, produced by nuclear RNA Pol III to form the RNA in the nucleus where the Drosha/Microprocessor endonuclease complexes are found (Gregory et al., Nature 432: 235-40, 2004; Han et al., Genes Dev 18: 3016-27, 2004), allowing Drosha to process the transcript into a shRNA or pre-miRNA, in principle allowing it then to enter the endogenous nuclear export pathway to the cytoplasm, where it is further processed by Dicer and used to program the RISC. The transcription that occurs in the cytoplasm is more desirable, however, because Drosha processing is more efficient when long ss flanking sequences extend from the base of the RNA stem-loop, requiring more costly larger DNA circles. Coligo 19adcr1 is an example of a circle designed to produce a dicer substrate.

Unlike rolling circle transcription, which requires a specific nucleotide or nucleotide sequence where cleavage is to take place, the endogenous pathway requires no specific sequence because it recognizes the stem loop secondary structure, not its sequence per se. This requirement enables it to recognize the many endogenous (genomically encoded) primary-miRNA it must process (as well as unlimited artificial sequences designed to have the appropriate secondary structure).

The methodology disclosed herein is premised on the activity of a mammalian RNAP III, which can be made available in the form of a substantially purified preparation of a mammalian RNAP III, cell extract from mammalian cells, or mammalian cells themselves. Hence, the present methodology allows for the production of RNA molecules in vitro (in test tubes or cell cultures), in situ (in tissue samples) and in vivo (in a mammal).

The term “mammal” and “mammalian” refer to any mammalian subject, including but not limited to human, murine, rat, bovine, feline, canine, and the like.

In order to utilize cells' endogenous RNAP III activity, DNA templates are introduced into mammalian cells using any of the art recognized gene delivery approaches, particularly non-viral delivery approaches, including but not limited to physical means and chemical means. Physical means can directly introduce the template DNA molecules into the cells, a target tissue, or the body fluids, the blood stream, such as microinjection, gene gun, particle delivery system (including biolistic particle delivery system and DNA-coated gold particles), electroporation, impalefection (delivery using nano-materials or nano-structure), sonication, and the like. Chemical means typically involve combining a DNA with a compound which facilitates the uptake of the molecule by cells, then introducing the molecule into the body fluids, the blood stream, or a selected tissue site. Suitable compounds include lipid compounds such as liposome, lipofectins, cytofectins, lipid-based positive ions, and the like. Other DNA carriers which can facilitate the uptake of a desired vector by the target cells include nuclear protein, or ligands for certain cell receptors. Additional modification to the template DNA can be made to achieve more effective cell penetration (e.g., by using a cell-penetrating DNA aptamer) or tissue-specific delivery (e.g., by using a catenated DNA circle linked with targeting molecules), as described above.

The amount of a DNA template introduced into mammalian cells can be controlled, depending upon the intracellular level of the RNA product desired. The DNA template can also be designed to control the level of RNA transcript produced, by optimizing the specific sequence in the large loop, such that the RNAs are not over-expressed to a level that would interfere non-specifically with the cell's metabolism or endogenous microRNA processing machinery.

### EXAMPLES

The present description is further illustrated by the following examples, which should not be construed as limiting in any way. The contents of all cited references (including literature references, issued patents, and published patent applications as cited throughout this application) are hereby expressly incorporated by reference.

### Example 1

### Circular Single-Stranded Synthetic DNA Delivery Vectors for MicroRNA

This Example describes experiments in which single-stranded (ss) circular oligodeoxynucleotides were designed to encode minimal primary miRNA mimics, with the goal of intracellular transcription in mammalian cells followed by RNA processing and maturation via endogenous pathways. Ss synthetic DNA templates were circularized by using a thermostable RNA ligase, which did not require a splint oligonucleotide to juxtapose the ligating ends. In vitro transcription of four templates demonstrates that the secondary structure inherent in miRNA-encoding vectors did not impair their rolling circle transcription (RCT) by RNA polymerases (RNAPs) previously shown to carry out RCT. A typical primary miRNA rolling circle transcript was accurately processed by a human Drosha immunoprecipitate.

Design and Synthesis of Circular DNA Templates Encoding miRNAs

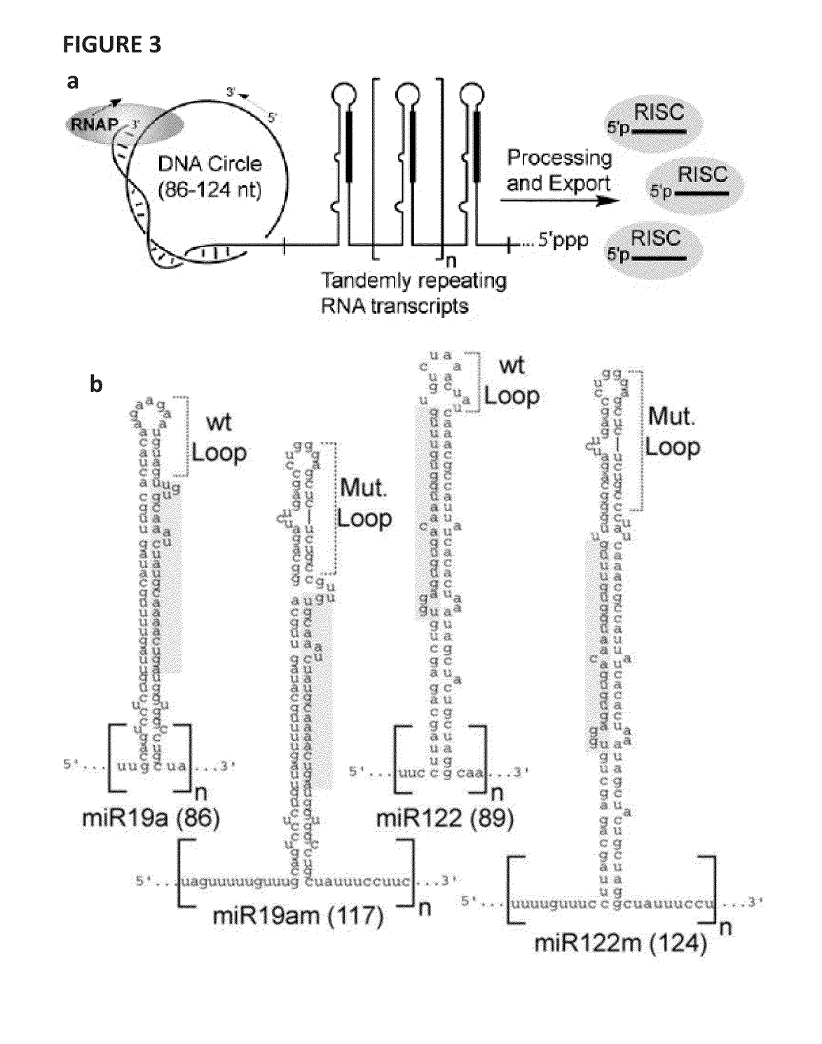

We designed DNA circles to encode shortened versions of two human primary miRNAs (pri-miRNAs), miR-19a and miR-122. We based our design on the endogenous human miRNA genomic template-strand sequences, and included DNA encoding ˜3 helical turns of the pri-miRNA stem-loop sequence (FIG. 3b), the minimum stem length required for Drosha/Microprocessor processing (ZENG et al., Embo J 24: 138-148 (2005); HAN et al., Cell 125: 887-901 (2006)). In two of the COLIGOs, we minimized the length of the sequences flanking the stem-loops to 4 nt (template 19a) and 6 nt (template 122) per repeat. In two other templates, 19 am and 122m, we included more of the natural flanking sequences (taken in both cases from the miR19a genomic template-strand sequence), for a total of 22 and 18 flanking nt, respectively. Additional flanking nucleotides (nt) are believed to enhance Drosha processing (HAN et al., Cell 125: 887-901 (2006); LEE et al., Nature 425: 415-419 (2003)). However, in a rolling circle transcript all but the terminal stem-loop repeats would be flanked by varying lengths of RNA, so it is not yet clear how much flanking RNA should be encoded by a given COLIGO. The complete monomer transcripts for the four templates—two for each miRNA—used in this study are shown in FIG. 3b.

Some miRNAs have conserved loops that aid in their processing (MICHLEWSKI et al., Mol Cell 32: 383-393 (2008)), but specific loop sequences are likely not critical to the biogenesis of most miRNAs (HAN et al., Cell 125: 887-901 (2006); ZENG et al., Rna 9: 112-123 (2003)). Loop sequences at the DNA level in the COLIGO, however, may influence transcription efficiency, as shown for E. coli RNAP RCT (OHMICHI et al., Proc Natl Acad Sci USA 99: 54-59 (2002)). We tested this variable, in combination with the longer flanking sequences, by swapping the native miRNA loops in miR19a and miR122 for an unrelated RNA hairpin loop (from HIV TAR RNA), resulting in miR19 am and miR122m (monomer transcripts are shown in FIG. 3b).

To choose the site where the linear oligonucleotide is closed to form the COLIGO, we predicted the folding of the COLIGO using mFold (ZUKER, Nucleic Acids Res 31: 3406-3415 (2003)) and selected the region having the least secondary structure, which in the linear form might prevent juxtaposition of the ends. This sequence typically coincided with the flanking nucleotides at the base of the stem (of the transcripts). The DNA circles encoding the transcripts shown in FIG. 3b were therefore ligated between the 5′ and 3′ deoxynucleotides corresponding to the 3′ and 5′ terminal ribonucleotides, respectively, depicted in the monomer transcripts (FIG. 3b).

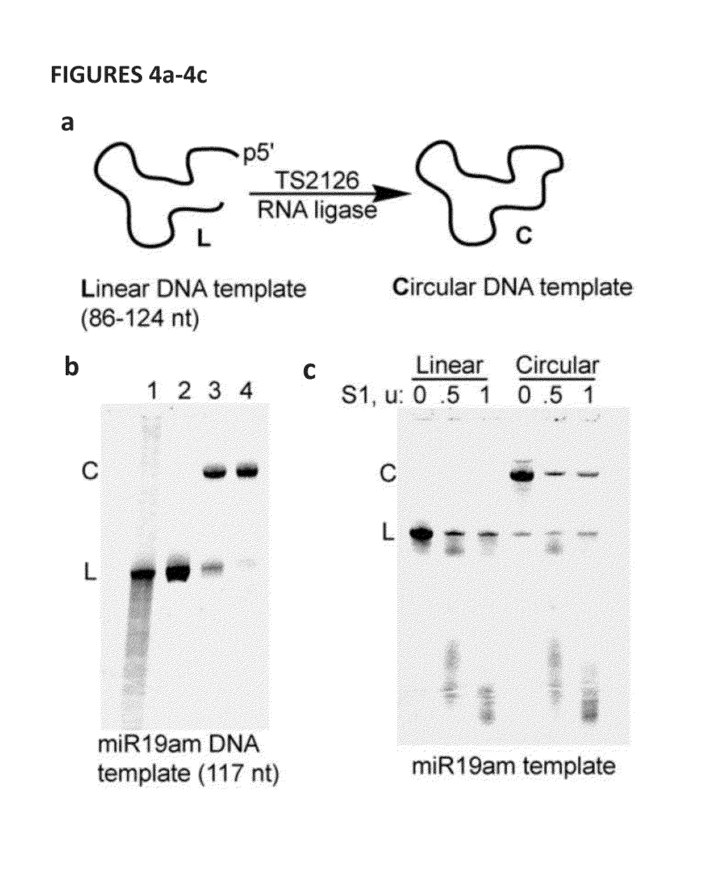

Rolling circle transcription templates have been circularized by three methods as previously reported: chemical ligation within a DNA triple-helix (DAUBENDIEK et al., Journal of Amer Chem Soc 117: 7818-7819 (1995)); T4 DNA ligase closure mediated by a splint oligonucleotide (DIEGELMAN et al., Protocols in Nucleic Acid Chemistry: 5.2.1-5.2.27 (2000)); and T4 DNA ligase closure within a nicked DNA dumbbell structure, as for the shRNA-encoding templates (SEYHAN et al., Oligonucleotides 16: 353-363 (2006)). We used a newly discovered thermostable RNA ligase (Rnl) from bacteriophage TS2126 (FIG. 4a). TS2126 Rnl has been reported to efficiently circularize ss DNA without a splint (BLONDAL et al., Nucleic Acids Res 33: 135-142 (2005)). In a typical COLIGO preparation using this enzyme, the entire 5′ phosphorylated linear template was made in one synthesis, gel purified by DPAGE and circularized with TS2126 Rnl. Most residual linear form was then removed by Exonuclease I treatment. For example, in the case of the 117 nt template encoding the miR19 am transcript (FIG. 3b), 5.0 nmol from the desalted DNA synthesis was gel purified to yield 1.2 nmol of the full-length form (23% full-length recovered). Following cyclization and Exonuclease I treatment, 0.87 nmol of the COLIGO was recovered (73% cyclization based on purified linear precursor). The gel profile of the DNA at the various stages is shown in FIG. 4b. A final prep-scale gel purification generally did not improve the circular-to-linear ratio, and led to a large loss of COLIGO. The circular topology of all COLIGOs used in this study was verified by S1 nuclease nicking (e.g. FIG. 4c), which for COLIGOs leads initially to the linear form with no intermediate gel bands (DIEGELMAN et al., Protocols in Nucleic Acid Chemistry: 5.2.1-5.2.27 (2000)).

In Vitro Transcription by Bacteriophage and Bacterial RNA Polymerases

While COLIGOs encoding shRNAs (SEYHAN et al., Oligonucleotides 16: 353-363 (2006)) and ribozymes (DIEGELMAN et al., Nucleic Acids Res 26: 3235-3241 (1998); DIEGELMAN et al., Chem Biol 6: 569-576 (1999); DAUBENDIEK et al., Nat Biotechnol 15: 273-277 (1997)) have been reported to undergo RCT in vitro by purified bacterial and bacteriophage RNAPs, COLIGOs encoding native pri-miRNAs have not been investigated prior to this disclosure. To determine whether native miRNA cDNA secondary structure might pose a general impediment to RCT, we exposed the four COLIGOs encoding miR19a and miR122 to T7 and E. coli RNAPs in vitro Transcripts were visualized by [α-³²P]-UTP incorporation. All four COLIGOs generated the large transcripts characteristic of RCT. For each template sequence, the circular topology was required to produce transcripts significantly longer than the circumference of the COLIGOs, and withholding one nucleotide triphosphate (ATP, Lane C—) led as expected to the loss of all but very short aborted transcripts. For all templates, more RNA was produced using E. coli RNAP compared to T7 RNAP under similar conditions (see Materials and Methods below). Denaturing formaldehyde agarose gel electrophoresis showed that the maximum length of the transcripts typically reached 5 kilobases (5 kb). Thus, all four DNA circles served as RCT templates despite the extensive secondary structure predicted for COLIGOs encoding pri-miRNA stem loop structures.

The relative amount of RNA produced in these experiments varied with the DNA sequence outside of the miRNA stem sequence. The template encoding the miR19a with the natural loop and short flanking sequence was especially poor, but it became a more efficient template when its wild-type (wt) loop was replaced with the TAR RNA loop and the flanking sequences were lengthened (compare 19a and 19 am). In contrast, the same loop and flanking sequences, when placed on the miR122 stem, decreased transcription efficiency slightly (compare 122 and 122m). Thus, the non-essential sequence regions of a pri-miRNA-encoding RCT template can influence, and be manipulated to modulate, the extent to which a COLIGO is transcribed.

Drosha/Microprocessor Processing of Rolling Circle Transcripts

Primary miRNA transcripts are first processed in their natural stem-loop context by Microprocessor, a complex that in mammalian cells includes Drosha and DGCR8 (HAN et al., Genes Dev 18: 3016-3027 (2004); GREGORY et al., Nature 432: 235-240 (2004)). We tested whether pre-miRNA hairpins could be processed from a multimeric rolling circle transcript. Treatment of RCT transcripts (made in vitro with uniform ³²P-labeling using E. coli RNAP) with HEK293T whole cell extract (WCE) led to the release of low levels of the pre-miRNA hairpin and the intervening flanking region. In order to test whether Drosha was responsible for this processing, we immunoprecipitated (IP'd) FLAG-tagged Drosha from transfected HEK293T cells (LEE et al., Nature 425: 415-419 (2003)). The activity of IP'd Drosha (with any co-IP'd proteins) was first verified on an in vitro transcribed human miRNA cluster (LEE et al., Embo J 21: 4663-4670 (2002)). When treated with the IP'd Drosha, the same processed RNAs were released from the multimer, but in higher amount. This result demonstrated that pre-miRNA hairpins can be accurately processed from RCT transcripts. If RCT were to take place in the nucleus of human cells, then some of the RNA should be capable of entering the natural processing pathway.

Materials and Methods

Materials, Reagents and General Procedures—

Synthetic DNA was made by Integrated DNA Technologies (Coralville, Iowa) and chemically 5′ phosphorylated. Preparative and analytical denaturing polyacrylamide gel electrophoresis (DPAGE) and denaturing agarose electrophoresis were done according to standard procedures [28]. DNA was located on preparative gels using UV shadowing over fluorescent silica gel plates (EMD Chemicals 5715-7). DNA was recovered by electroelution in 1×TBE within a sealed dialysis membrane tube with a molecular weight cut off of 1000 (Spectrum Laboratories). Eluted DNA was phenol-chloroform extracted and ethanol precipitated with sodium acetate. In some cases, the DNA gel slice was eluted in 0.3 M sodium-acetate at 37° C. overnight and ethanol precipitated. The 1% agarose gel was prepared using Ambion's 10× denaturing gel buffer, blotted overnight onto positively charged nitrocellulose (Ambion), baked for 20 min at 55° C., UV crosslinked and exposed to a PhosphorImager® screen (Molecular Dynamics). E. coli RNAP was purchased from USB. T7 RNAP and DNA ligase were purchased from New England Biolabs. RNA markers were purchased from Sigma (R4142) or Ambion (Decade marker, AM7778) and prepared according to the manufacturer's instructions. For the agarose gel used in analyzing the transcripts from miRNA-encoding DNA circles by E. coli and bacteriophage T7 RNA polymerases, sizes were determined by running 8 μg of total cellular RNA extracted from HEK293T cells on the same gel and staining for ribosomal RNA (18S=1.9 kb and 28S=5 kb) with methylene blue after blotting.

Synthesis of Linear DNA Templates—

Linear DNA templates encoding shortened forms of pri-miR19a, -miR122 and -miR19 am (e.g. FIG. 4b) were synthesized by IDT as single 5′ phosphorylated Ultramer sequences. The linear templates for miR122m and miR19 am were also made from two half-length oligonucleotides (e.g. 19 am-1 and 19 am-2) according to the standard splint mediated T4 DNA ligase procedure (DIEGELMAN et al., Protocols in Nucleic Acid Chemistry: 5.2.1-5.2.27 (2000)). The sequences used, including 19 am-1 (63 nt), 19 am-2 (54 nt), splint 19 am (30 nt), 122m-1 (65 nt), 122m-2 (59 nt), and splint 122m (31 nt) were set forth in the Sequence Listing (see Table).

TS2126 RNA Ligase I—

A synthetic gene (Top Gene Technologies Inc., Canada) encoding the first 393 amino acids of TS2126 RNA ligase (Rnl) I (BLONDAL et al., Nucleic Acids Res 33: 135-142 (2005)) followed by a C-terminal hexahistidine tag was inserted into the EcoRI/SmaI sites of pBluescriptSK(+) plasmid (Stratagene). The synthetic DNA contained codons optimized for expression in E. coli. The entire coding sequence of this plasmid, pTS2126H, was verified by DNA sequencing. E. coli B121-CodonPlus(DE3)-RIL (Stratagene) was transformed with pTS2126H and grown in 2 liters of culture. At O_{D595} ˜0.5 the culture was induced to a final concentration of 1 mM IPTG and grown for another 3 hrs at 37° C. The cells were pelleted at 3000 g for 15 min at 4° C., resuspended in 30 ml native lysis buffer (50 mM Na_{H2}P_{O4}. 300 mM NaCl, 10 mM imidazole. pH 8 with NaOH), and disrupted using a French press. After centrifugation to remove debris, the lysate was incubated with 1 ml Ni-NTA agarose (Qiagen) with agitation for 2 hrs at 4° C. The beads were washed three times with 5 ml native lysis buffer each time, once with elution buffer without imidazole (20 mM Tris-HCl pH 8, 100 mM KCl, 2 mM DTT) and finally eluted with 500 μl portions of elution buffer containing 250 mM imidazole. The pooled fractions containing the protein were dialyzed (3 times, 2 hrs each) against 10 mM Tris-HCl (pH 8), 50 mM KCl, 0.1 mM EDTA and 1 mM DTT, and stored at 4° C. (short term) or in 50% glycerol and −80° C. (long term).

Circularization of Oligonucleotides—

Reactions contained a ratio of 0.015 nmol linear 5′ phosphorylated DNA (0.75 μM final conc.) to 1.5 μg TS2126 Rnl and the following components (final concentrations): 50 μM ATP, 2.5 mM MnCl₂, 50 mM MOPS (pH 7.5), 10 mM KCl, 5 mM MgCl₂ and 1 mM DTT. These conditions were used for reactions ranging from 0.015 to 1.5 nmol. Circularizations were incubated for 2 hrs at 60° C., followed by phenol/chloroform extraction and ethanol precipitation. In preliminary syntheses, the circularized products and unreacted linear templates were separated on a 1.5 mm DPAGE and visualized by UV shadowing, excised from the gel and electroeluted. In cases where the COLIGO was still contaminated by >5% of the linear oligonucleotide after elution (as determined by gel staining), an Exonuclease I (NEB) digest was done. For most COLIGOs used in this study, we used the more recently adopted procedure shown in FIG. 4b. The crude desalted oligonucleotide was gel-purified, circularized as described above, phenol/cholorform extracted and precipitated. The COLIGO was then treated with 10 u of Exonuclease I per 0.1 nmol DNA in a total volume of 50 μl according to the manufacturer's instructions, phenol/cholorform extracted, precipitated and used without further gel-purification after analytical scale DPAGE showed no multimeric circles and less than 5% linear form by 0.05% Stains-All (Acros) staining.

S1-Nuclease Assay—

To verify the circular topology of the COLIGOs (as in FIG. 4c), the following procedure was used: a 10 μl reaction containing 1 μg of linear or circular oligonucleotide and 0, 0.5 or 1 u of S1 nuclease (USB) in 50 mM sodium acetate, pH 4.6, 1 mM zinc acetate, 250 mM NaCl and 0.5 μg BSA was incubated for 10 min at 37° C., extracted with phenol/chloroform, ethanol precipitated, separated by 10 or 12% DPAGE and visualized with Stains-All.

In Vitro Transcription—

The following conditions were used for in vitro transcription with bacteriophage T7 and E. coli RNAPs: final template concentration 1 μM, 1 unit/μl RNAse inhibitor (Promega), 0.5 mM each NTP, ˜2 μCi [α-³²P]-UTP; and (for T7 RNAP: 40 mM Tris-HCl pH 7.9, 6 mM MgCl₂, 10 mM DTT, 2 mM spermidine); (for E. coli: 40 mM Tris-HCl, pH 8.0, 10 mM MgCl₂, 5 mM DTT, 50 mM KCl, 50 μg/ml BSA). Each 10 μl reaction contained the following amount of RNAP: 40 units T7, 1 unit E. coli. Reactions were incubated for 1 hr at 37° C., after which time the RNA was extracted with 150 μl TriReagent (Ambion), isopropanol precipitated according to the manufacturer's instructions with 10 μg glycogen added. For all in vitro RNAP reactions, radiolabeled transcripts were separated on a 9% denaturing polyacrylamide gel, dried and exposed to a PhosphorImager® screen.

Drosha Immunoprecipitation and In Vitro RNA Processing—

The pCK-Drosha-FLAG plasmid (kindly provided by V. N. Kim) was expressed and purified as previously described (LEE et al., Nature 425: 415-419 (2003)). Briefly, 8 μg of plasmid were transfected per 6 cm dish of HEK293T cells using 20 μl Lipofectamine 2000 (Invitrogen). After 44 hrs, the cells were lysed (900 μl Sigma FLAG® kit lysis buffer/6 cm dish) and Flag-IP was carried out using 40 μl of a 50% anti-FLAG bead slurry (Sigma FLAG® Tagged Protein Immunoprecipitation Kit) for at least 4 hrs at 4° C. The beads were washed 4 times with 1× Wash Buffer and once in processing buffer (20 mM Hepes-KOH, pH 7.9, 100 mM KCl, 0.2 mM EDTA, 0.5 mM DTT, 0.2 mM PMSF, 5% glycerol). A parallel mock-IP was carried out using Lipofectamine without the Drosha-encoding plasmid. The activity of the immunoprecipitated protein was verified using the miRNA-23-27-24-2 cluster transcribed in vitro from plasmid pGEM-T-easy_pri-miR-23, 27, 24-2(+) and (−) (also provided by V. N. Kim). For HEK293T WCE as the Drosha source, cells were lysed as described above; the crude lysate was agitated at 4° C. for 30 min, centrifuged for 30 min at 13000 rpm and the supernatant collected and adjusted to 20% glycerol. For in vitro processing of rolling circle transcripts from circle 19 am, 1/10 of an in vitro transcription reaction using E. coli RNA polymerase (described above) was used in a total volume of 30 μl containing 15 μl beads from Drosha Flag-IP (or mock-IP, or 15 μl WCE), processing buffer including 6.4 mM final MgCl₂ and 0.1 u/μl RNase inhibitor. The processing reaction was carried out for 90 min at 37° C. RNA was extracted with TriReagent (Ambion), isopropanol precipitated and separated on a 10% denaturing polyacrylamide gel, dried and exposed to a PhosphorImager® screen.

### Example 2

### Circularized Synthetic Oligodeoxynucleotides Function as RNA Polymerase III Templates for Small RNA Production in Human Cells

This example describes experiments which demonstrated that circularizing a DNA encoding a general pre-miRNA stem-loop structure triggered its circumtranscription by human RNAP III. While transfected DNA circles permeate cells, their transcripts were found mainly in the cytosol, suggesting they were made there by the promoter-independent RNAP III activity associated with innate immunity.

Coligo Transcription: Processivity and Evolutionary Complexity

To investigate whether human RNAPs also carry out RCT, we designed a coligo to code for a minimized primary (pri)-miR-122 stem-loop RNA (FIG. 5a). In the form of rolling circle transcripts made by bacteriophage or E. coli RNAP (FIG. 5a, n=large number), coligo transcripts fold into tandemly arrayed multimers resembling naturally occurring pri-miRNA from clustered, polycistronic miRNA genes.

Coligo 122 (FIG. 5a) was made by circularizing a synthetic 89 nt genomic sequence encompassing human miR-122's non-coding strand (see Example 1). FIG. 5b shows an in vitro transcription (IVT) comparison of 122 with RNAPs from bacteria, yeast (RNAP II) and mammals (HEK293T whole cell extract, WCE). A comparison of lanes 3, 6 and 9 reveals a distinct trend in which coligo transcription processivity, and hence RCT efficiency, declined with the evolutionary progression of the RNAPs tested. RCT is recognized by the characteristic pattern of large transcripts that enter the denaturing polyacrylamide gel (DPAGE) without migrating further (DAUBENDIEK et al., Journal of Amer. Chem. Soc. 117, 7818-7819 (1995)) (FIG. 5b, gel region “a”). As a relative measure of transcriptional processivity, we calculated the ratio of the radioactivity incorporated in the rolling circle transcripts (FIG. 5b, gel region “a” in lanes 3, 6, 9) to the sum of all shorter transcripts (gel region “b”). Lower a/b ratios indicate lower processivity. By this measurement, yeast RNAP II showed approximately 8-fold lower RCT processivity than E. coli RNAP (2.3 vs. 0.3) using coligo 122. Collectively, the human RNAPs were another 30-fold less processive, with an a/b ratio of only 0.01, and showed primarily single round transcription (i.e., FIG. 5a, n ˜1). Thus, the ability to initiate promoterless transcription on coligo templates appears to have been retained during RNAP evolution, while the processivity needed for RCT appears to have been lost.

Precise Coligo Transcription by a Human RNA Polymerase

Though no RCT was observed, transcription of coligo 122 by the human WCE was remarkable for having produced a single, well-defined and relatively abundant in vitro transcript, indicating transcription initiation and termination at specific sites. The size similarity between coligo 122 (89 nt) and its transcript (˜83 nt) indicated that the initiation and termination sites are close to one another. About 4% of transcription events read through one time and terminated on the second pass, producing a dimer transcript at ˜460 nt. Although no RCT treadmill action on the coligo was needed to produce the single round, or monomer, transcript, the circular topology of the coligo sequence was important, as no transcript was produced from the linear (non-circularized) template (FIG. 5b, lane 8 vs. 9). Thus, the circular topology of coligo 122 appeared to promote transcription by one or more human RNAPs, and termination occurred non-randomly just upstream of the site of initiation.

In an initial test for generality, we made three additional coligos (FIG. 5c) similar in size and design to 122 (see Example 1). FIG. 5d shows the RNA products formed during IVT in WCE using these templates in both linear (L) and circular (C) form. Coligo 19a was a poor substrate, but 19aTAR, which contains the same 19a miRNA stem, templated the synthesis of three resolvable and relatively abundant transcripts. Coligo 122 TAR, which contains the 122 stem but different loops, produced six resolvable transcripts. As observed for 122, the transcripts from 19aTAR and 122TAR were slightly shorter than their template, low-levels of dimer transcripts resulted from read-through, and the linear forms did not template productive transcription. Taken together, these results show that single round transcription is not peculiar to coligo 122, and that sequence variations outside of the miRNA-encoding stem determine transcription efficiency and the sites of initiation and termination.

Adding linkers to, and sequencing the cDNA of, the ˜115 nt 19aTAR transcripts revealed a single start site (13/13 clones) in the larger ss loop (Tss1, FIG. 5e). (A second start site, Tss2, was also found in a 5′ RACE procedure. See the Methods second hereinbelow). About half ( 6/13) of the cloned transcripts terminated precisely at a dC just upstream of Tss1. This termination site immediately preceded an A₅ (AAAAA, SEQ ID NO: 68) run, a sequence that in a ds DNA context is a strong RNAP III termination signal (COZZARELLI et al., Cell 34, 829-35 (1983)). All other transcripts terminated either just before or just after this dC. Next, 5′ and 3′ RACE procedures were used to sequence the ˜83 nt 122 transcript (FIG. 15f). This transcript also began exclusively in the larger of the two terminal loops of the coligo and, similar to the 19aTAR Tss1 case, mainly at one site near the helix stem (FIG. 5f, Tss, 23/27 clones). Transcription termination of coligo 122 occurred just after a known RNAP III termination signal (AAACA, SEQ ID NO: 69) (ORIOLI et al., Nucleic Acids Res 39, 5499-5512). Thus, though wholly synthetic, ss and lacking a promoter, coligos 19aTAR and 122 both underwent precise single round (i.e. one time around the coligo) transcription initiation to produce stable stem-loop RNAs having surprisingly well defined 3′ ends.

Coligo Transcription is General and Produces Dicer Substrates

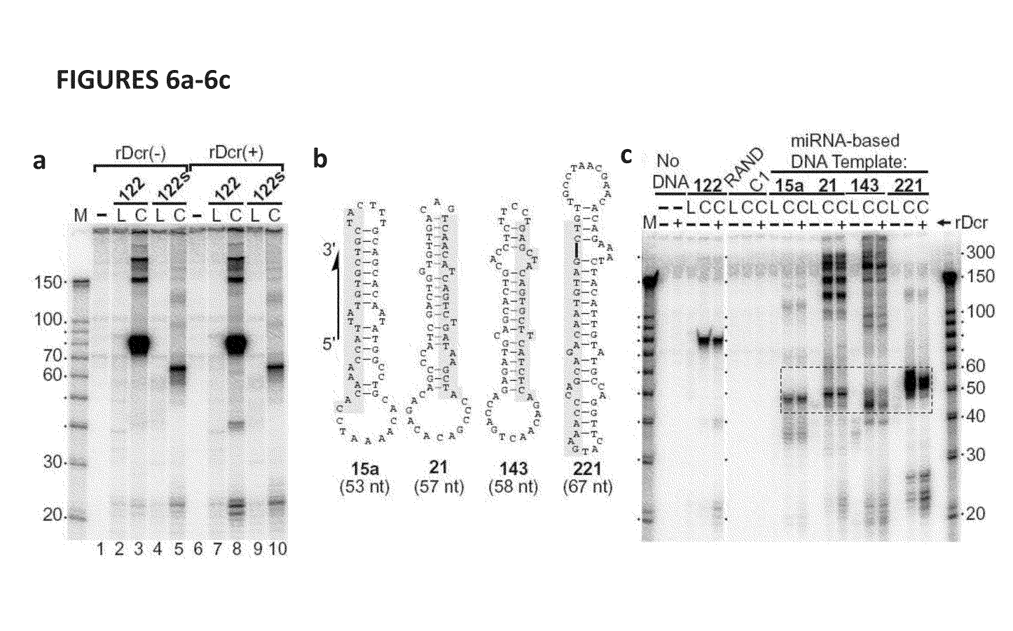

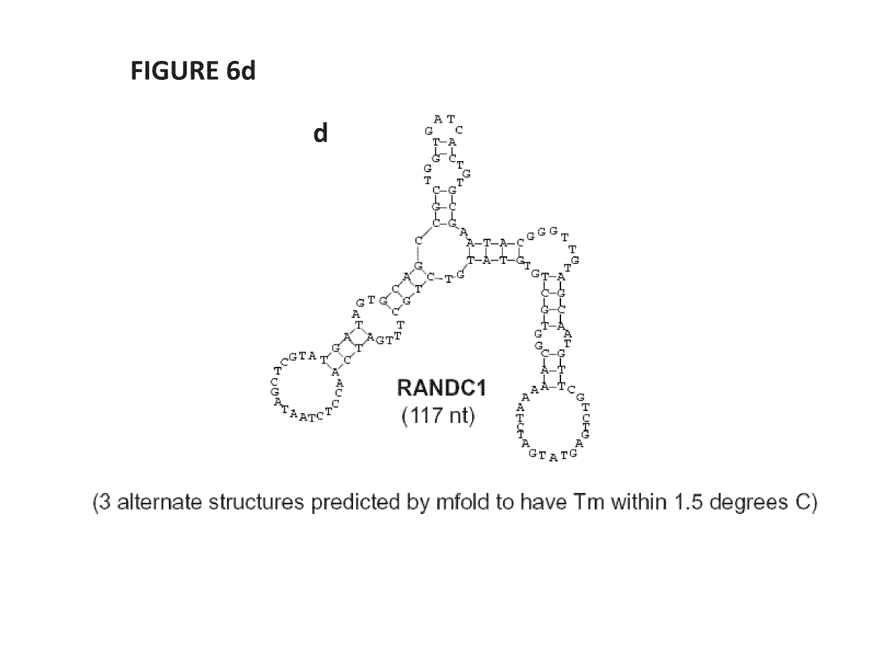

Coligos 19a and 122TAR were initially designed to lead to RCT products suitable for Drosha processing. The single round transcription products we unexpectedly found had too little flanking sequences to be good Drosha substrates (HAN et al., Cell 125, 887-901 (2006)) and stems that were too long to mimic natural pre-miRNA Dicer substrates. Nevertheless, a long gel exposure of coligo 122 IVT showed possible Dicer processing products at 21-24 nt (FIG. 6a, lane 3). We shortened 122 to produce coligo 122s, a template whose transcript should better mimic natural pre-miRNA (122s, FIG. 1g). 122s templated the 65-nt transcript predicted by analogy to 122 and, although transcribed to a lesser extent, led to a greater fraction of putative Dicer products (FIG. 6a, lane 5). In both cases, 21-24 nt RNAs increased in amount and proportion when IVT was done using WCE from cells transiently over-expressing recombinant Dicer (lanes 6-10), supporting the possibility they resulted from Dicer processing rather than aborted transcription. To further test the generality of coligo transcription and Dicer processing, we designed similarly sized coligos for 4 randomly chosen human miRNAs (miR-15a, -21, -143, -221; FIG. 6b) and treated them with WCE from untransfected or Dicer-transfected HEK293T cells. Along with these we tested a coligo with the same size and G+C content as 19aTAR but no ability to template a stem-loop RNA (RANDC1, FIG. 6d). Each stem-loop encoding coligo produced monomer size transcripts (FIG. 6c, boxed) and more abundant 21-24 nt RNAs in WCE from Dicer over-expressing cells, while RANDC1 produced no transcripts. Coligos 21 and 143 were notable for having no more than 2 consecutive A's, making them poor RNAP III termination templates, perhaps explaining why they also produced more oligomeric transcripts (up to 300 nt) than templates having 3-5 A's. These results indicate that (1) coligos encoding pre-miRNA stem-loop mimics are general substrates for a human RNAP that prefers to transcribe once around the template; and (2) even when lacking the preferred 3′ dinucleotide Dicer substrate overhang, Dicer can process the transcripts into 21-24 nt RNAs in vitro.

Coligos Lead to Biologically Relevant Transcript Levels

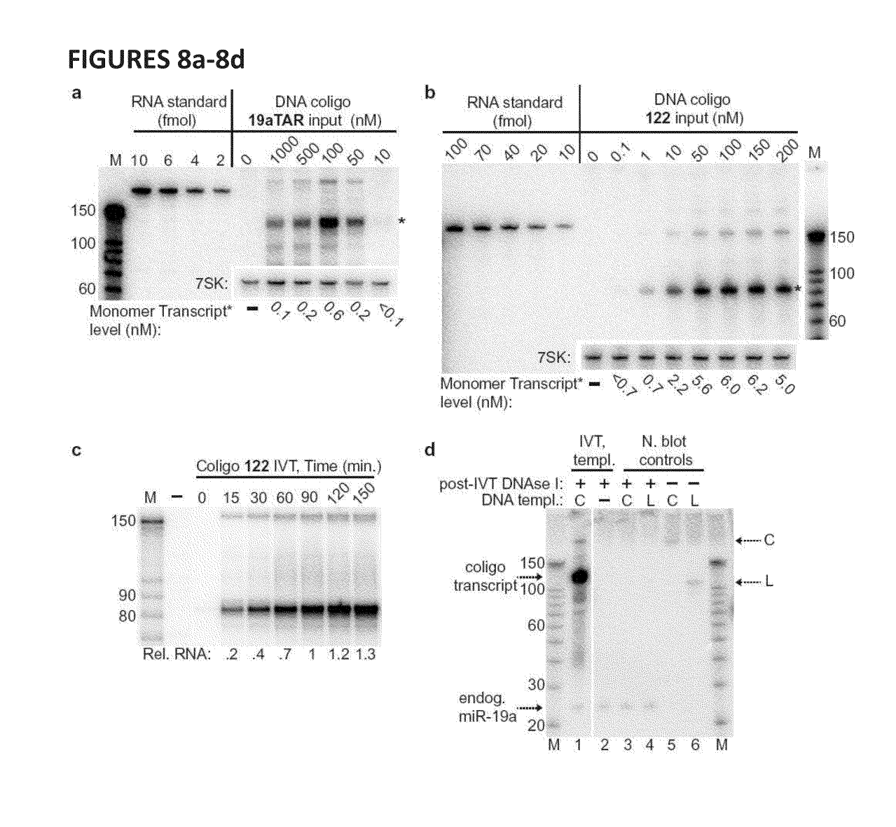

We used quantitative Northern blotting to estimate the amount of single round transcripts formed as a function of coligo concentration (FIGS. 8a,b). Single round transcripts from 19aTAR were detectable using as little as 10 nM coligo, and increased to an RNA concentration of ˜0.6 nM transcribed from 100 nM coligo. Raising the coligo concentration eventually reduced the amount of transcript. In the case of coligo 122, single round transcripts were detectable at 100 μM coligo, and a plateau of ˜6 nM transcript was reached at 50 nM coligo. The RNA levels measured here were for 90 minute transcription reactions, but RNA levels continued to increase up to 2.5 hr, the longest time point monitored (FIG. 8c). High concentrations of RNA may therefore be achievable from very low coligo concentrations over longer periods of exposure to active RNAPs.

To put coligo transcript levels into perspective, we compared the single round transcript level from 19aTAR to mature miR-19a in HEK293T extract. Based on RNA-Seq experiments in the related HEK293 cell line under various conditions, miR-19a is moderately to highly expressed in HEK cells (LANDGRAF et al., Cell 129, 1401-14, Table S12 (2007)). Endogenous miR-19a in the HEK293T whole cell extract (FIG. 8d) was visualized using a standard LNA Northern probe which visualizes both transcripts with the same stoichiometry. The coligo 19aTAR transcript at 90 min was ˜65-fold higher than miR-19a in WCE. This result demonstrated that, in vitro, coligo templates can in a short time produce significantly more transcript than a typically expressed endogenous miRNA.

Coligos and Transcript are Stable to Cellular Nucleases, but do not Remain Hybridized.

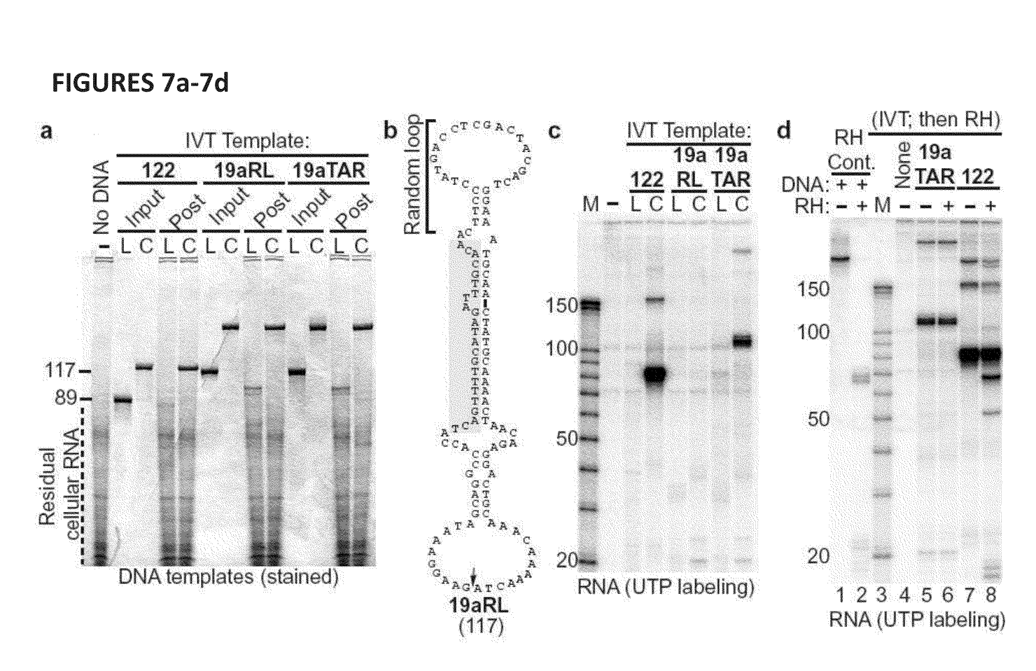

FIGS. 1 and 2 demonstrate that the coligo's circular topology promotes productive transcription, even though—and in contrast to RCT—no treadmill action should be required to produce single round transcripts. To test whether transcribability correlated with stability against cellular nucleases, we attempted to recover linear and coligo templates at the end of a WCE IVT reaction. FIG. 7a shows that while coligos were stable in HEK293T WCE, their linear forms were degraded. Coligo 19aRL (FIG. 7b), where the TAR loop of 19aTAR was replaced by an unstructured loop, was stable but not transcribed (FIG. 7c). Thus, a circular topology alone is not sufficient for an oligonucleotide to undergo specific transcription; coligo loop sequence, size and structure also determine transcribability.

Our data show that both coligo and transcript were stable in WCE. Promoterless transcription from 3′ tailed templates can lead to a round of transcription ending with a stable RNA:DNA hybrid (DEDRICK et al., Biochemistry 24, 2245-53 (1985)). While the fact that stable coligo transcripts are observed argues against the presence of an RNA:DNA hybrid, whose RNA would be subject to cellular RNase H degradation (WU et al., Antisense Nucleic Acid Drug Dev 8, 53-61 (1998)), we nevertheless tested the possibility of persistent RNA:DNA hybrids by adding recombinant RNase H to an IVT reaction (FIG. 7d). Validated RNase H activity (lanes 1 and 2) had no effect on the 19aTAR transcripts, while ˜20% of the 122 transcripts were susceptible to added RNase H. We conclude that the strong intramolecular secondary structure of coligo and transcript prevent significant coligo:transcript hybrid formation. This result also rules out the possibility that the RNAP terminates after single round transcription because it encounters and cannot unwind a persisting RNA:DNA hybrid at the transcription start site.

Coligo Transcription in Human Cells

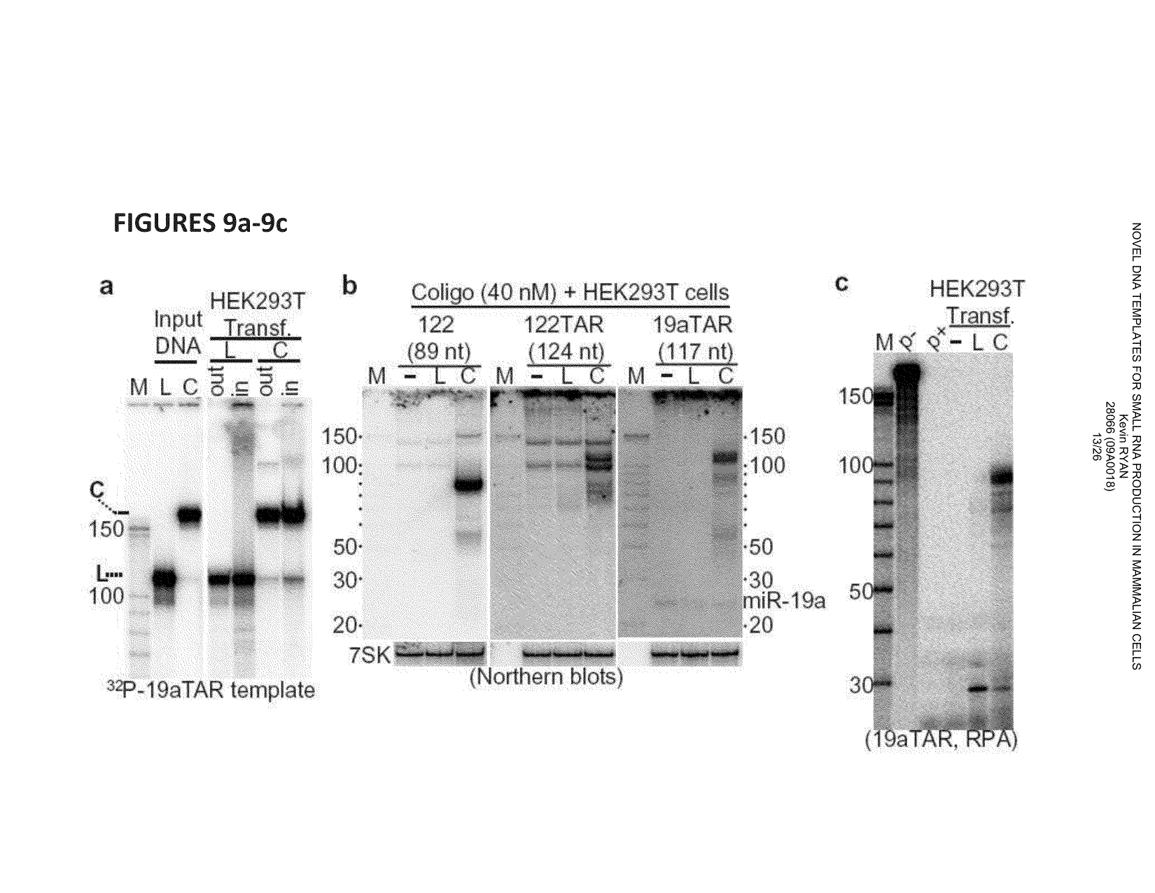

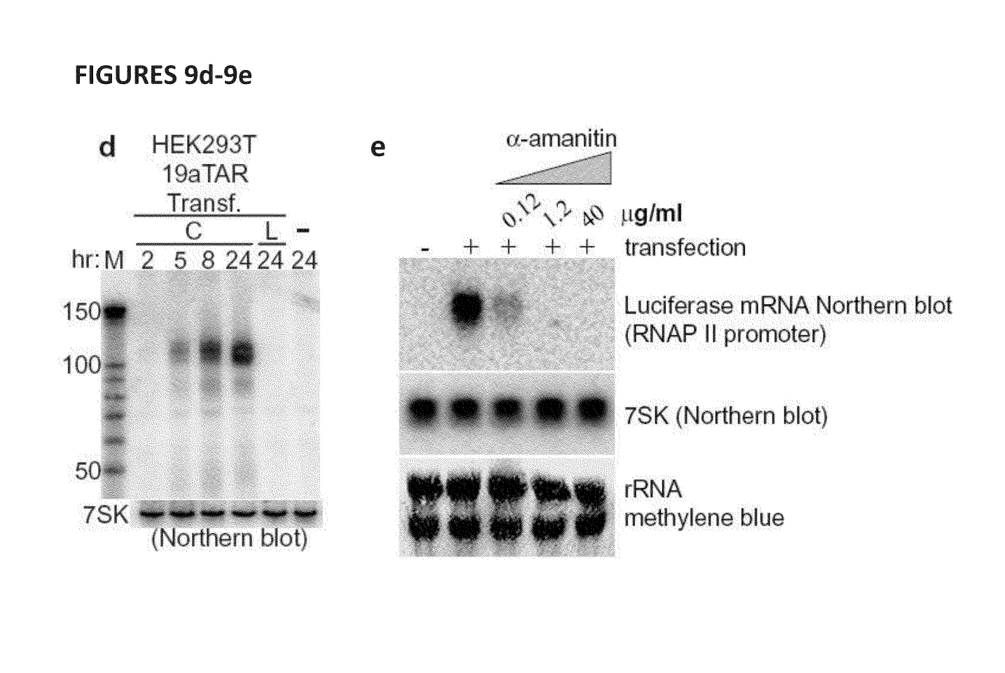

We next asked whether coligos can enter and undergo transcription in human cells. Coligo 19aTAR and its linear counterpart were transfected into HEK293T cells at 40 nM. Including ³²P-labeled tracers of the same DNA revealed that ˜60% of the linear and coligo templates entered the cells and remained stable during a 24 hr experiment (FIG. 9a). Three coligos that were successfully transcribed in vitro were transfected into HEK293T cells and assayed by Northern blotting of total cellular RNA after 24 hours. In each case, transcripts the sizes of those seen in vitro were detected (FIG. 9b). After 24 hours transfection, the 19aTAR transcript was found at 26-fold greater abundance than the mature miR-19a (FIG. 9b, right panel). Though apparently stabilized by the transfection reagent (FIG. 9a), the linear form produced no detectable transcripts. The importance of the circular topology in vivo was further supported by an RNase protection assay (RPA) performed on the total RNA isolated from transfected cells (FIG. 9c). Here, the temperature of the assay was lower than the Northern hybridization temperature, enabling the detection of aborted transcripts. Both the linear and coligo forms of 19aTAR templated a small fragment at ˜30 nt, but only the coligo underwent circumtranscription. A time course furthermore showed that the 19aTAR transcript continued to increase over the 24 hr period (FIG. 9d). Overall, FIG. 9 shows that transcription in cells was well-represented by transcription in vitro: a circular topology was important, single-round transcripts slightly smaller than the coligo were made, and the coligos templated the accumulation of stable transcripts over time. Thus, coligo IVT faithfully reconstituted intracellular transcription.

Coligos are Transcribed In Vitro and Intracellularly by RNAP III

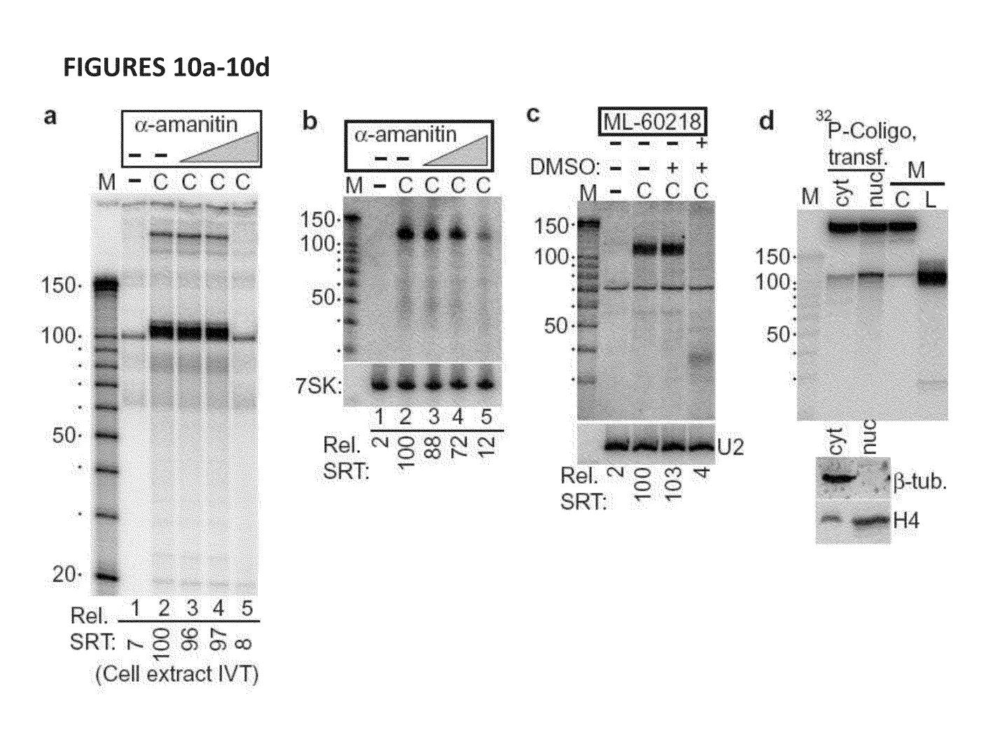

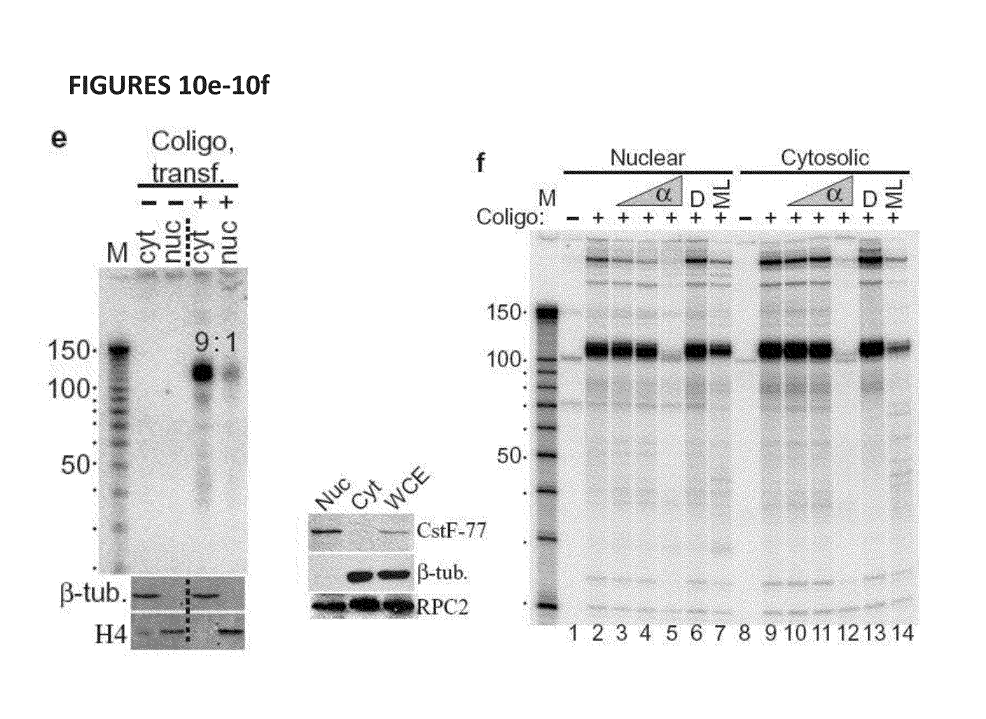

To learn which of the mammalian RNAPs is responsible for coligo transcription, we took advantage of their different susceptibilities to the transcription inhibitor α-amanitin (SCHWARTZ et al., Journal of Biol Chem 249, 5889-97 (1974)). IVT of coligo 19aTAR was carried out using HEK293T WCE in the presence of increasing concentrations of α-amanitin. At 0.12 μg/ml, a concentration which only inhibits RNAP II (SCHWARTZ et al., Journal of Biol Chem 249, 5889-97 (1974)) (FIG. 9e), coligo transcription was unaffected, ruling out RNAP II (FIG. 10a). At 120 μg/ml, which inhibits RNAP III but not RNAP I, no coligo transcripts were produced, implicating RNAP III. Similar results were obtained when coligo 19aTAR was transfected into HEK293T cells in the presence of increasing amounts of α-amanitin, followed by Northern blotting of total cellular RNA with a 19aTAR-specific probe (FIG. 10b). To further test the identity of the RNAP, we treated HEK293T cells with ML-60218, an RNAP III-specific inhibitor (WU et al., Eukaryot Cell 2, 256-64 (2003)). Northern blotting showed that no single round transcripts were produced in ML-60218-treated cells transfected with coligo 19aTAR (FIG. 10c). These results demonstrated that RNAP III is responsible for producing all single round transcripts from coligo templates in vitro and in human cells.

Coligos May Engage the Innate Immune System to Undergo Transcription

Promoter-independent RNAP III activity was recently found in the cytosol of HEK293 cells, where it's transcription of poly(dA:dT) revealed an unexpected role for the polymerase in the innate immune system (ABLASSER et al., Nat Immunol 10, 1065-72 (2009); CHIU et al., Cell 138, 576-91 (2009)). RNAP III was found to transcribe polyd(A-T), a viral DNA surrogate, into ds RNA, which in turn activated an interferon response through the RIG-I helicase. Because coligos also undergo promoterless transcription, and because A/T-rich DNA is prone to forming transient loops that might resemble A-rich coligo loops, we investigated whether coligos could be transcribed in the cytosol by engaging this natural process. We therefore tracked the sub-cellular location of the coligo and its transcripts. A radiolabeled coligo was transfected into HEK293T cells and found to be evenly distributed between nuclear and cytosolic fractions (FIG. 10d). In contrast, Northern blotting of fractionated extracts from coligo 19aTAR-transfected cells showed that 90% of the coligo transcripts were in the cytosolic fraction (FIG. 10e). To verify that the cytosol contained RNAP III capable of coligo transcription, we fractionated untransfected HEK293T WCE, verified that significant amounts of RNAP III was present in both nuclear and cytosolic fractions, (RPC2 Western blotting, inset, FIG. 10f), and carried out IVT with and without RNAP III inhibitors. The results showed clearly that cytosolic RNAP III carried out strong coligo transcription identical to the pattern generated by WCE (FIG. 10f). Nuclear RNAP III also transcribed coligos (FIG. 10f). While we cannot rule out the possibility that the cytosolic transcripts in FIG. 10e were made in the nucleus and exported to the cytosol, until a promoter-independent role for RNAP III in the nucleus is found, the simpler explanation is that the coligo was transcribed mainly by RNAP III in the cytosol. These data support the possibility that coligos are primarily transcribed in the cytosol through engagement of RNAP III's pathogen recognition role in innate immunity.

Materials, Reagents and General Procedures.

Synthetic DNA Ultramer® oligonucleotides were made with 5′ phosphorylation by Integrated DNA Technologies (Coralville, Iowa). Enzymatic DNA cyclization using the TS2126 RNA ligase, coligo purification, RNA marker, denaturing polyacrylamide gel electrophoresis (DPAGE) were done as described in Example 1. In some cases, when the crude Ultramers® were sufficiently free of failure sequences to cyclize without prior gel purification, the DPAGE step was done after cyclization, where it removes any unreacted linear form and the (circular or linear) failure sequences, obviating the need for a final exonuclease treatment. RNA secondary structures were predicted using the on-line version of the mfold program (Zuker, Nucleic Acids Res 31, 3406-15 (2003)). E. coli RNAP was purchased from USB. Yeast RNAP II was provided by D. Bushnell and R. Kornberg (Cramer et al., Science 292, 1863-76 (2001)). General molecular biology methods were done following standard procedures (see, e.g., Sambrook, J. & Russell, D., Molecular cloning: A laboratory manual, Cold Spring Harbor Press, Cold Spring Harbor, N.Y., 2001). DNA sequencing was done by Macrogen.

Cell Culture and Preparation of Whole Cell Extracts (WCE) with or without Recombinant Dicer.

In a typical procedure, four 10 cm dishes of confluent HEK293T cells grown in 1×DMEM supplemented with 10% fetal calf serum and Penn./Strep. were rinsed three times with cold 1×PBS on ice. 350 μl to 700 μl lysis buffer (50 mM Tris-HCl pH 7.4, 150 mM NaCl, 1 mM EDTA, 1% Triton X-100 and Protease Inhibitor Cocktail, Roche (cat. #04693159001) was added per dish and incubated on ice for 5 min. Lysed cells were scraped to one side of the dish and transferred with a 1 ml pipette tip to pre-chilled 1.5 ml microfuge tubes. Cell-lysis was continued for 5-10 more min on ice until the DNA was visibly precipitating. The extract was spun at 4° C. for 10 min at 13.2 krpm to pellet genomic DNA and other insoluble materials. The supernatant was transferred to a new chilled tube, adjusted to a final glycerol concentration of 20% v/v, aliquoted and stored at ˜80° C. The protein concentration was determined by Bradford assay (BioRad cat #500-0006) and ranged from 2 μg/μl to 6 μg/μl depending on the amount of lysis buffer used. For preparation of extract enriched in Dicer protein used in FIGS. 6a and c, a plasmid encoding the Dicer protein (pFRT/TO/FLAG/HA-DEST DICER, Addgene 19881) was transfected into 6 cm dishes HEK293T cells using Lipofectamine according to the manufacturer's instructions. Cell extracts were prepared 48 hrs after transfection with the method described above. A non-transfected control using Lipofectamine only was processed in parallel, and used for Dcr−.

### Preparation of Cytosolic and Nuclear Extracts for IVT

Different methods were used to obtain satisfactory cytosolic and nuclear fractionation. In order to obtain the highest cleanliness of each fraction (<5% contamination of material from one fraction in the other) cytosolic and nuclear fractions were prepared from separate cell populations. These extracts were used for IVT in FIG. 10f.

The nuclear fraction was prepared as follows: 3×10 cm dishes confluent HEK293T were washed 3× with ice cold PBS and the dishes were placed on ice. 500 μl of fractionation buffer (10 mM HEPES-KOH pH 7.9, 1.5 mM MgCl₂, 10 mM KCl, 0.5 mM DTT, 0.04% NP-40, Protease Inhibitor cocktail) were added to each 10 cm dish, the cells were scraped to one side of the dish and transferred with a 1 ml pipette tip to 15 ml conical tubes. Cells were incubated on ice for 10 min and spun at 4° C. for 6 min at 2000 rpm. The supernatant was discarded; the nuclei were washed twice with cold 1 ml 0.5× fractionation buffer and pelleted in between washes as above. The pellet was resuspended in 200 μl fractionation buffer adjusted to 0.3 M NaCl, incubated on ice for 10 min with occasional vortexing. Nuclei were disrupted by dounce homogenization using a tight fitting, pre-chilled type A pestle for 20 strokes. The debris was spun down for 20 min, 13 krpm at 4° C., the supernatant (nuclear fraction) was transferred to a new pre-chilled tube and adjusted to 15% Glycerol. Aliquots were snap frozen in liquid nitrogen and stored at ˜80° C.

Cytosolic fraction was prepared as follows: initial steps are same as for the nuclear fraction but after resuspension in fractionation buffer the cells were monitored for lysis under a light microscope until around 50% of the cells had lysed. This reduced the total amount of protein but prevented significant contamination of the cytosolic fraction by lysed nuclei. Sufficient lysis was observed in less than five minutes after which the fraction was spun down for 1 min at 13 krpm at 4° C. to pellet nuclei, the supernatant was transferred to another pre-chilled tube and spun for an additional 15 min at 13 krpm 4° C. to pellet residual cellular debris. The supernatant after this spin, designated cytosolic extract, was adjusted to 15% Glycerol and 0.3 M NaCl (to adjust the salt concentration to that of the nuclear fraction), snap-frozen in aliquots and stored at ˜80° C.

Cytosolic-Nuclear Fractionation for Coligo and Transcript Location.

In order to directly compare cytosolic to nuclear fractions for experiments in FIGS. 10d,e, separations were carried out using Fermentas' ProteoJET™ kit (K0311) according to the manufacturer's instructions. Briefly, cells were harvested by trypsinization, washed twice with cold PBS and the pellet was lysed with 10 packed cell volumes. The cells were lysed on ice for 5 min, the nuclei were spun down at 700 rcf for 5 min, the supernatant was transferred to a new pre-chilled 1.5 ml tube and the nuclei were washed twice with 500 μl nuclei wash buffer. The nuclei were resuspended in 240 μl nuclei storage buffer and lysed by addition of 10 μl nuclear lysis buffer, kept on ice for 10 min with intermittent vortexing. Both cytosolic and nuclear fractions were spun to collect debris for 20 min (4° C., 13 krpm) the extract was transferred to a new pre-chilled tube, the final volume was adjusted to 15% Glycerol, snap frozen and stored at ˜80° C.

The cleanliness of separation using the ProteoJET™ kit varied from <10% to up to 30% nuclear leakage (i.e. contamination of the cytosolic fraction with nuclear proteins) in the lysis step. Similar results were obtained by using Ambion's PARIS™ kit (AM1921) according to the manufacturer's instructions. A method utilizing 40 μg/ml Digitonin in RSB-100 buffer (10 mM Tris-HCl, pH 7.4, 100 mM NaCl, 2.5 mM MgCl₂) resulted in a clean nuclear preparation but significant contamination of the cytosolic fraction with nuclear material, similar to the two kit-methods described above. In the digitonin method, cells were washed three times with ice cold 1×PBS and scraped with 500 μl RSB100/Digitonin into pre-chilled 15 ml Falcon tubes. The cells were spun at 2800 rpm in a swinging rotor at 4° C. for 5 min, the supernatant was designated as the cytosolic extract and the nuclei were washed with 1 ml RSB100/Digitonin buffer and spun as above. The wash was decanted and nuclei were lysed in fractionation buffer (10 mM HEPES-KOH pH 7.9, 1.5 mM MgCl₂, 10 mM KCl, 0.5 mM DTT, 0.04% NP-40, 0.3 M NaCl, Protease Inhibitor cocktail), broken by dounce homogenization with 20 strokes of a tight fitting pestle A and the debris was spun down at 13.2 krpm at 4° C. for 10 min. The supernatant (nuclear fraction) was adjusted to 15% glycerol, snap frozen and stored at −80° C.

In vitro transcription (IVT). IVT using WCE, uniform labeling with [α-³²P]-UTP and the linear or circular (coligo) templates was performed as follows. A typical 20 μl reaction contained 25 μg total WCE protein, 20 units RNase inhibitor (Promega) 1.25 mM each ATP, CTP, GTP, 0.2 mM UTP, (except FIG. 5b, which contained 1.25 mM each NTP), ˜2 μCi [α-³²P]-UTP, 40 mM Tris-HCl pH 7.9, 6 mM MgCl₂, 10 mM DTT, 2 mM spermidine, 100 μM NaCl); 100 nM template unless otherwise indicated. IVT using E. coli RNA polymerase (USB) was carried out in 40 mM Tris-HCl, pH 8.0, 10 mM MgCl₂, 5 mM DTT, 50 mM KCl, 50 μg/ml BSA), 1 μM coligo template, 1.25 mM NTP, ˜2 μCi [α-³²P]-UTP, 1 unit/μl RNAse inhibitor. IVT with yeast RNAP II was performed with 2.5 μg purified enzyme in 40 mM Tris-HCl pH 7.9, 6 mM MgCl₂, 10 mM DTT, 2 mM spermidine, 1.25 mM NTP with 1 μM coligo template. Transcription reactions were incubated for 90 min at 37° C., after which time the RNA was extracted with 150 μl TriReagent (Invitrogen) per 20 μl reaction volume, according to the manufacturer's instructions, with 10 μg glycogen added. Radiolabeled transcripts were separated over 9% DPAGE, dried on Whatman paper and exposed to a Molecular Dynamics Phosphorimager Screen. Images and quantitation were performed using MD ImageQuant software. In FIG. 5b, the optimal coligo concentration for each RNAP was used to show each enzyme at its most processive.

Template Recovery from IVT Reactions.

In FIG. 7a, each reaction was scaled up 4-fold to allow for visualization of the templates by Stains-All dye. 1 μl of a 1/10 dilution of an RNase A/T1 mix (RNase cocktail, Ambion) was added to each reaction and incubated for 30 additional min at 37° C. Nucleic acids were isolated by phenol/chloroform/isoamyl alcohol (PCI) extraction and ethanol precipitation. After DPAGE, the gel was soaked for 5 min in ddH₂O and stained (1 mg Stains-All plus 1.25 ml formamide diluted to 20 ml with ddH₂O) for 30 min, destained in ddH₂O under incandescent light, dried on Whatman paper and scanned.

Generation of Probes and Standards for Quantitative Northern Blotting.

Standards used in FIGS. 8a,b were produced by IVT from plasmids p122 and p19aTar, encoding the respective coligo transcript sequences under the control of a T3 RNAP promoter (T7 for the antisense transcript) and contained the full length sequence of the ss DNA coligos cloned into pBluescript II SK after PCR with primers containing BamH I and EcoRI restriction sites. The plasmids were transcribed by T3 RNA polymerase in 100 μl using 2 μg of linearized plasmid and 50 units of T3 RNA polymerase (Promega) according to the T3 manufacturer's protocol. For FIG. 8a (19aTAR), the plasmid-derived transcript was excised from a 6% DPAGE gel and eluted for 3 hrs at 37° C. into RNA elution buffer (0.5 M NH₄OAc, 1 mM EDTA, 0.2% SDS). For FIG. 8b, at the end of a 3 hour IVT reaction using the linearized plasmid, 3 units of DNase I were added and the incubation was continued 30 min at 37° C., PCI extracted and ethanol precipitated. Quantification of RNA in both cases after resuspension was done by (1) absorbance at 260 nm (in 8 M urea) and (2) a comparison, on a stained 9% DPAGE gel, with ss DNA oligonucleotides of known concentration. Radioactively labeled antisense Northern probes used to detect transcript levels in FIGS. 9c,d, FIGS. 10b,c,e and FIGS. 8a,b were generated by T7 RNAP (40 units) from linearized p122 and p19aTar and isolated from an excised gel slice as described above. IVT was carried out for 1 hr at 37° C. in 40 mM Tris-HCl pH 7.9, 6 mM MgCl₂, 10 mM DTT, 2 mM spermidine; 0.6 mM each ATP, CTP, GTP, 0.1 mM UTP, plus ˜2 μCi [α-32P]-UTP, 1 unit/μl RNAse inhibitor, followed by PCI and ethanol precipitation.

RNase H Probing.

At the end of a 90 min IVT, 1 unit of RNase H (Promega) was added and the incubation was continued for an additional hour. For the RNase H positive control lane, the linear form of coligo 19aTAR was mixed with the p19aTAR T3 run-off transcript, annealed by heating 4 min in 93° C. followed by slow-cooling. Isolated total RNA from the same amount of WCE used in the IVT reaction was added to normalize non-specific competition, and RNAse H was added as above. RNA was isolated by extraction with TriReagent, resolved by DPAGE and exposed to a Phosphorimager screen.

RNase Protection Assay (RPA).

The probe was generated by [α-³²P]-UTP T7 IVT from p19aTAR transcription as described above, excised and eluted from 6% DPAGE, PCI extracted and ethanol precipitated. For hybridization, 1/20 of the eluted probe was precipitated together with 12 μg RNA prepared from linear or circular 19aTAR transfected HEK293T cells, or untransfected cells, and hybridized in 10 μl RPA hybridization buffer (Ambion AM1415) at 55° C. overnight. RNA was then digested with an RNAse A/T1 cocktail in 150 μl RNase digestion buffer according to the manufacturer's instructions for 30 min at 37° C. The RNA was ethanol precipitated with 1 μg glycogen, and separated over 10% DPAGE. 1/20 of the undigested input probe (p− in FIG. 9c) or the probe digested without cellular RNA (p+ in FIG. 9c) were loaded for reference. The probe's T7 5′ start site and 3′ end are offset from the coligo transcript's 3′ and 5′ ends (judged by sequencing results shown in FIG. 3e), leading to a protected fragment that is smaller than 19aTAR's normal ˜110 nt IVT transcript.

Transfection of HEK293T Cells and Northern Blotting.

Twelve hours before transfection, HEK293T cells were seeded in 12-well plates (1 ml 1×DMEM with 10% FBS) then grown to 90% confluency. 46 μl of 1×DMEM was mixed with 4 μl of 10 μM template stock in TE, 10 μl PolyFect (Qiagen) were added and complexed for 8 min at room temperature. The non-transfected control contained 46 μl 1×DMEM, 4 μl TE and 10 μl PolyFect. 700 μl of the media was removed from each well and the DNA complexes were added with 640 μl of fresh 1×DMEM supplemented with 10% FBS (without Penn./Strep). After 24 hrs (or other indicated times, FIG. 9d), RNA was harvested by lysing the cells of each well with 800 μl TriReagent and isolating the RNA according to the manufacturer's (Invitrogen) instructions. The pellet was resuspended in 50 μl 1× DNase I buffer, 2 μl were removed for OD₂₆₀ measurement and the RNA was digested with 4 units of DNase I for 1 hr at 37° C. 12 μg of RNA were ethanol precipitated and separated over 10% DPAGE (0.75 mm gel thickness; 0.4 for clearer bands but greater difficulty), blotted onto positively charged BrightStar®-Plus Positively Charged Nylon Membrane (Ambion AM10102), baked for 20 min at 55° C., UV-crosslinked, pre-hybridized in Church hybridization buffer (0.25 M Na₂HPO₄, 7% SDS, 1 mM EDTA), and hybridized in the same buffer at 55° C. overnight to a uniformly labeled RNA probe transcribed from the appropriate linearized plasmid (p19aTAR in FIGS. 9d, 10b,c,e, and FIG. 8a, or p122 in FIG. 8b). For the LNA Northern blots shown in FIG. 9b and FIG. 8d, 20 pmol of the 122 or 19a LNA (Exiquon), complementary to the mature 122 and 19a hsa-miRNAs, were 5′ end-labeled with [γ-³²P]-ATP/T4 Polynucleotide kinase, and used in place of the uniformly labeled probe described above. LNA hybridization and washing were done also as described above, except at 42° C. Final Northern blots were washed twice for 30 min each with Church wash buffer (1% SDS, 20 mM Na₂HPO₄) and exposed to a PhosphorImager screen (Molecular Dynamics).

For RNA produced by IVT, RNA was extracted using 150 μl Trizol/20 μl IVT volume. The RNA was treated with DNase I (NEB) for 1 hr, Ethanol precipitated and blotted as described above. As loading control, the blot was re-hybridized to a 5′ [γ-³²P]-ATP end-labeled DNA oligonucleotide (5′-TGGACCTTGAGAGCTTGTTTGGAGGTT, SEQ ID NO: 52) complementary to a portion of the endogenous 7SK RNA sequence (FIGS. 9b,d, 10b, FIGS. 8a,b and FIG. 9) or U2 snRNA for FIG. 10c (5′-AGCTCCTATTCCATCTCCCTGCTC, SEQ ID NO: 53). 20 pmoles of end-labeled DNA oligo were used for hybridization overnight at 45° C. For FIG. 8d, lanes 3 and 4, the 19aTAR template was added to WCE and processed immediately as described above but without IVT incubation; for lanes 5 and 6, the 19aTAR templates were electrophoresed without extraction or DNase I treatment, and show the full Northern blot signal of the DNA templates if no DNase I treatment is used.

α-Amanitin Activity Verification (FIG. 9e).

10 μg of total RNA from pMir-Report (Ambion) transfected HEK293T cells was separated over a 1.2% agarose gel (2.2M formaldehyde, 20 mM MOPS, 2 mM NaOAc, 1 mM EDTA), blotted by capillary transfer over night to Bright Start Plus membrane (Ambion), stained with methylene blue to visualize ribosomal bands and hybridized as described above to a [γ-³²P]-ATP end-labeled oligonucleotide detecting luciferase mRNA (5′-TCGTCTTCGTCCCAGTAAGCTATGTCTCCAGAATGTAGCCA, SEQ ID NO: 54) at 45° C., and reprobed subsequently using the 7SK RNA probe. 0.12 μg/ml α-amanitin equals 5× the RNAP II IC₅₀—(See Schwartz et al., J Biol Chem 249, 5889-97 (1974)). The RNAP III IC₅₀ is 20 μg/ml α-amanitin. RNAP III is not detectably inhibited by 0.5 μg/ml α-amanitin (Schwartz et al., supra). RNAP I is not detectably inhibited by 400 μg/ml α-amanitin (Schwartz et al., supra).

19aTAR RNA Whole Transcript Sequencing.

A typical coligo 19aTAR IVT was scaled up 10-fold (200 μl 250 μg WCE, 100 nM coligo, no 32P label). Total RNA was recovered using TriZol, resuspended in 150 μl annealing buffer (20 mM Tris-HCl, pH 8.4, 50 mM KCl, 6 mM MgCl₂). A 5′ RACE experiment (see below) had shown that transcripts contained the intact TAR loop. To enrich the eluted fraction for the 19aTAR transcripts over the abundant endogenous HEK293T RNA, a biotin-streptavidin selection was carried out using a 5′ Biotin labeled ss DNA oligonucleotide complementary to the TAR RNA sequence (5′Biotin CGGCAGAGAGCTCCCAGGCTCAGATCTGCC-3′, SEQ ID NO: 55). Streptavidin-coated beads (Dynabeads MyOne Streptavidin Cl, Invitrogen) were prepared as follows: to remove any RNAse contamination on the beads, 60 μl of 50% beads slurry were washed twice for 2 minutes each with 200 μl 100 mM NaOH/50 mM NaCl in DEPC treated ddH₂O. The beads were then washed 3× with 500 μl 100 mM NaCl. The beads were resuspended in 150 μl 2× binding/washing buffer (10 mM Tris-HCl pH 7.4, 1 mM EDTA, 2 M NaCl). The RNA solution was added to 50 pmol of the 5′Bio-oligo, heated to 93° C. for 4 min, and allowed to cool to room temperature. The nucleic acids were added to the previously washed beads in 2× binding buffer to produce the 1× binding medium. The mix was incubated at room temperature with gentle rocking for 30 min. The beads were magnetically concentrated, the supernatant removed, and the beads washed 5× with 500 μl 1× binding buffer. To elute the selected RNA from the beads, the reaction was PCI extracted. The aqueous phase was ethanol precipitated with 10 μg glycogen. The selected RNA was purified using 6% DPAGE. The gel region between 90-130 nt was excised and eluted overnight in 800 μl gel elution buffer at 37° C. The eluted RNA was PCI extracted, ethanol precipitated with glycogen and resuspended in ddH₂O. The 3′ adaptor ligation was performed without ATP using 900 ng pre-adenylated Universal miRNA Cloning Linker (NEB) and 100 units of truncated T4 RNA ligase 2 (NEB) in T4 RNA ligase buffer with 12% PEG 8000, in a total volume of 20 μl for 2 hrs at 37° C. To remove unreacted adaptor, the reaction was PCI extracted, ethanol precipitated, resolved using 6% DPAGE and the gel slice between 80-150 nt was eluted as described above. Two units of Calf Alkaline Phosphatase (CIP, Promega) were used in case the RNA has triphosphate at the 5′ end. The RNA was PCI extracted, ethanol precipitated and resuspended in water. The 5′ end was phosphorylated using ATP and T4 PNK. The 5′ adaptor ligation was carried out using Ambion's RLM-RACE kit according to the manufacturer's instructions. Briefly, 300 ng of the 5′ RNA adaptor were ligated to the eluted, 3′ adaptor-containing RNA using T4 RNA ligase 1 in T4 RNA ligase buffer. 3 μl of this reaction were used in a reverse transcription reaction with 30 pmol of MiRclon3NotI oligonucleotide for 1 hr at 55° C. using SuperScript III Reverse Transcriptase (Invitrogen). The reaction was stopped by heating to 75° C. for 15 min. 1 unit RNAse H was added for 30 min at 37° C. and 1 μl of this reaction was used for nested PCR amplification using the 5′ outer primer and MiRclon3NotI reverse primer for 30 cycles. Inner PCR was performed with the 5′ inner primer and MiRclon3NotI primer for 35 cycles. The resulting PCR product was purified on 2% agarose; the band around 200 bp was excised and eluted using an agarose gel extraction kit (GenScript). The eluted PCR product was digested with the restriction enzymes. The digested PCR was again gel-purified, eluted, recovered and ligated into pBluescriptII SK previously digested with the same restriction enzymes and gel purified at a molar ratio of 3:1 (insert:vector) at 16° C. overnight using T4 DNA ligase with 1 mM final ATP in a 20 μl reaction. 5 μl of the ligation were transformed into 75 μl competent E. coli and the bacteria were plated onto LB-Amp plates supplemented with 40 μl each of 20 mg/ml X-Gal and 100 mM IPTG to allow for blue-white selection. White colonies were grown and miniprep plasmids were analyzed for inserts using the same restriction enzymes used for subcloning the insert. 34 insert-containing clones were picked and sequenced by Macrogen; 13 contained inserts originating from 19aTAR transcripts (FIG. 5e), the rest contained ribosomal RNA sequences.

19aTAR RLM-RACE 5′ End Sequencing.

RNA was generated in a 10-fold scale up of an unlabeled IVT reaction. The gel slice between 80-150 nt from a 6% DPAGE was excised and eluted overnight in elution buffer. The RNA was treated with CIP, 5′ phosphorylated with PNK and joined to the 5′ adaptor as described above. Reverse transcription was performed using random decamers, followed by PCR using the 5′ outer primer and a 19aTAR transcript-specific primer (19aT-fwd see below) for 30 cycles. 1 μl of this reaction was used in a nested PCR with primers 5′ inner primer and 19aT-fwd (see below). The PCR products were separated on 2% agarose, the product at ˜150 bp was eluted, digested with BamHI and cloned into pBluescript II SK as described above. Out of 15 clones, 8 began with Tss1 and 7 began with Tss2. In combination with the length of the IVT transcripts, the 5′ RACE allowed us to determine that the TAR loop was present in the full length transcripts represented by the 5′ RACE hits, and this fact was used in the biotin selection. Tss2 was not found in the full length sequencing. These shorter transcripts or their cDNA could have been lost during one of the gel purifications.

DEAE25 Treatment of WCE.

DEAE-sephadex A-25 (Amersham Biosciences) was used to partially remove short RNA species from the WCE. DEAE beads were washed five times with lysis buffer and 100 μl of a 50% suspension of beads was added to 500 μl WCE. The suspension was gently rotated for 30 min at 4° C. and the procedure repeated using a new aliquot of DEAE beads. This WCE was used in IVT of coligo 122 for 5′ RACE-RLM.

122 RLM-RACE 5′ End Sequencing.

Two procedures were used. First procedure. 250 μg protein from a DEAE-treated WCE was used in a 200 μl, [α-³²P]-UTP labeled IVT with 100 nM coligo 122. The extracted RNA was resolved using 6% DPAGE and the gel was exposed to film for 2 hrs. The aligned film was used to locate and excise the monomer transcript (−83 nt) which was eluted overnight in gel elution buffer at 37° C. To trim any possible 5′ triphosphates to 5′ monophosphate, the RNA was treated with 20 u of RNA 5′ polyphosphatase (Epicentre). The 5′ RACE adaptor ligation was done as described above and resuspended in 25 μl. 3 μl were used for reverse transcription with random decamers at 50° C. for 1 hr with the MMLV RT from Ambion's RLM-RACE kit in 25 μl. 1 μl was then used in outer PCR for 30 cycles with 5′ outer primer and 5end122-6. Inner PCR was performed with 5′ inner primer and 5end122-5 for 35 cycles. The PCR products between 60 and 100 bp were isolated from a 3% agarose gel and 1/10 of the eluted product was re-amplified for 30 cycles using the same primers as for inner PCR. The reaction was digested with BamHI and NotI, re-purified over a 3% agarose gel and the eluted fragment was quantitated by UV absorbance at 260 nm. Cloning was performed in a 3:1 (insert:vector) ratio as described above. Plasmids were purified from 3 ml over night culture in LB-Amp and ˜7 μg plasmid were digested and examined by 10% native PAGE to check for inserts. Positive clones were sequenced by Macrogen. Second procedure. Performed as above except that the coligo transcript-specific primer was located farther downstream of the transcription start site as determined by the first RACE experiment. Briefly, 1 μl of the RT reaction ( 1/25th) was amplified with 5′ inner primer and 5end122-2, the band around 100 bp was eluted from a 3% agarose gel and 1/10 of the eluted product was re-amplified using the same primers. The resulting product was digested with BamHI and NotI and cloned into pBluescript II SK as described above. Similar results were found in both experiments. 30 out of 30 positive clones contained 122 transcript cDNA and 27 are depicted in FIG. 5f. Three related clones began 4 nt away at the -TTT- sequence in the stem and contained either an additional -a- or -acac- at the 5′ end, which we assume to be artifacts of the sequencing procedure.

122 RLM-RACE 3′ end sequencing.

RNA from a 10-fold scale up coligo 122 IVT reaction containing [α-³²P]-UTP was extracted with TriReagent, isopropanol precipitated and separated by 10% DPAGE. The single band at ˜80-85 nt was excised and eluted overnight in elution buffer. The RNA was PCI extracted and precipitated as above. A poly(A) tail was added to the 3′ end using 5 u E. coli poly(A) polymerase (NEB, #M0276S) in a 20 μl reaction plus 1 mM ATP for 1.5 hrs at 37° C. The RNA was PCI extracted, precipitated, and used as the template for reverse transcription using Ambion's 3′RACE adapter and ThermoScript Reverse Transcriptase at 55° C. for 1 hr. Sequence specific PCR was carried out using the 3′RACE Inner primer and 122aE3end2 in a 100 μl reaction for 35 cycles. The resulting PCR product was PCI extracted, ethanol precipitated, digested with 20 units BamHI, extracted, precipitated and separated over a 3% agarose gel. The band ˜150 bp was isolated and ligated at a 3:1 (insert:vector) ratio into BamHI cut and Calf Alkaline Phosphatase treated pBluescript II SK as described above. 80 μl competent E. coli cells were transformed with 6 μl ligation mix, grown on LB-Amp plates and white colonies were picked for plasmid miniprep. 19 out of the 19 positive clones were found to carry an insert originating from the 122 transcript (FIG. 5f). Although the RNA sequences for the coligo 122 single round transcripts shown in FIG. 5 are described as “composite,” the 3′ RLM-RACE procedure actually sequenced up to the third nucleotide from the 5′ end of the dominant transcript (found in 23 clones), or 98% of the entire transcript. This resulted from our choice of the primer binding site on the reverse transcriptase reaction product, i.e. primer 122aE3end2 (see below). The 5′ RLM-RACE was required to know the extreme 5′ ends. Lastly, four 3′ RACE clones ended with UGU, as indicated in FIG. 5f, but had one or two untemplated, non-A nt between the templated 3′ end and the added poly(A) tail, which we assumed to be an artifact of the sequencing procedure, and are not counted in FIG. 5f.

Primers used in sequencing procedures are set forth in SEQ ID NOS: 56-67.

Use of Inhibitors α-Amanitin and ML-60218 in IVT and tf.

The RNAP III specific inhibitor ML-60218 (EMD Biosciences) (IC₅₀ for human RNAP III is 27 μM) was resuspended in DMSO at final concentration of 135 mM and stored at ˜20° C. Just prior to use, the stock was diluted to 9 mM and 67% DMSO with ddH₂O. Only under this dilution could ML-60218 be added to an IVT reaction or transfection without immediate precipitation of the inhibitor. α-amanitin (Sigma-Aldrich) was diluted in ddH₂O to 1 mg/ml and stored at ˜20° C.

For transfection experiments, the inhibitor was added to the cells together with the PolyFect-DNA complex and incubated for 9 hrs to minimize cell death by prolonged exposure to the poison. Concentrations were as follows: α-amanitin; low=0.12 μg/ml, medium=1.2 μg/ml, high=40 μg/ml; ML60218: 67.5 μM (˜2×IC₅₀ RNAPIII).

For IVT: the inhibitor was added to a standard IVT setup at the start-point of incubation at the following concentrations: low=0.12 μg/ml, medium=1.2 μg/ml, high=120 μg/ml or 225 μM ML-60218. For both transfection and IVT with ML-60218 a control reaction was included using the same final percentage of DMSO only (1.1% in IVT, 0.35% in transfection).

For FIG. 9e, plasmid pMIR-REPORT™ (expressing luciferase mRNA under CMV promoter, Ambion #AM5795M) was transfected in 12 well plates in HEK293T cells using 1.6 μg plasmid and 4 μl lipofectamine per well according to the manufacturer's instructions. Both plasmid and lipofectamine were diluted in Optimem media (Invitrogen 11058-021). α-Amanitin was added at the time-point of transfection together with the plasmid-lipofectamine complexes and incubated for 14 hrs.

IVT using cytosolic and nuclear extracts with inhibitors: equal volumes (8 μl) of cytosolic (5 mg/ml total protein) and nuclear extracts (3 mg/ml total protein) were used in IVT (standard conditions) including inhibitors or solvent (as control, water or DMSO) in the reaction setup. We note that the inhibitory effect of ML-60218 in IVT reactions appeared to be sensitive to the buffer (or other condition) used for extract preparation, possibly causing less effective inhibition of transcription in IVT reactions, while it was consistently >90% in transfection experiments.

Production of Isotopically Labeled Coligo (FIG. 5d).

100 pmoles of 5′ phosphate containing oligo 19aTAR were treated with 3 units calf alkaline phosphatase for 1 hr at 37° C. to remove the 5′ phosphate. After PCI extraction and ethanol precipitation, the oligo was [γ-³²P]-ATP end-labeled with 20 units PNK enzyme and 2 μl [γ-³²P]-ATP (6000 Ci/mmol) and incubated for 1 hr at 37° C. The oligo was PCI extracted and precipitated. 75% of the end-labeled oligo was circularized as described in Example 1. The ligation reaction and the unligated linear oligo were separated over 7% DPAGE and eluted overnight in 0.3 M NaCl at room temperature. After elution, the oligos were PCI extracted, ethanol precipitated, washed in cold 75% Ethanol and resuspended in 10 μl TE buffer.

Transfection for Transcript Location and Coligo Location.

In order to separate cytosolic and nuclear fractions, transfections were carried out in 6 cm cell culture dishes using 200 pmoles of coligo or linear form (final concentration of 40 nM) and 35 μl PolyFect. For transfection of isotopically labeled oligos, approximately 5 pmoles of template labeling reaction product were transfected together with the unlabeled oligos.

Western Blots.

10% SDS-PAGE (7% for RNA polymerase III large subunit RPC2) was used for electrophoresis of nuclear and cytosolic fractions (same volume as used for IVT reaction shown in same panel) and blotted on nitrocellulose membrane. Blots were blocked for 1 hr in 3% non-fat dry milk in PBS. Incubation with antibodies was performed in PBS+0.1% Tween-20 (PBST). Primary antibodies were incubated over night at 4° C., secondary antibodies for 2 hrs at room temperature. Primary antibodies were purchased from Novus Biologicals (CSTF3, H00001479-M01), Santa Cruz Biotechnology (β-tubulin sc-80011), Bethyl (H4 histone A300-646A and RPC2, A301-855A, subunit of RNAPIII). Secondary antibodies were from Calbiochem (goat anti-rabbit IgG Peroxidase Conjugate DC03L) and Santa Cruz Biotechnology (goat anti-mouse IgG₁-HRP, sc-2060). Dilutions of primary and secondary antibodies were made according to the manufacturer's instructions. Blots were developed using ECL system (GE Healthcare).

Oligo Sequences Used for Circularization. See Table 1.

Patents, patent applications, publications, product descriptions, and protocols which are cited throughout this application are incorporated herein by reference in their entireties. The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

### SEQUENCE LISTING

The instant application contains a Sequence Listing which has been submitted in ASCII format via EFS-Web and is hereby incorporated by reference in its entirety. Table 1 lists all sequences disclosed herein.

TABLE 1

| | | | |
|---|---|---|---|
| SEQ | | | Structure of coligo |
| ID | Description | Sequence | or RNA transcript |

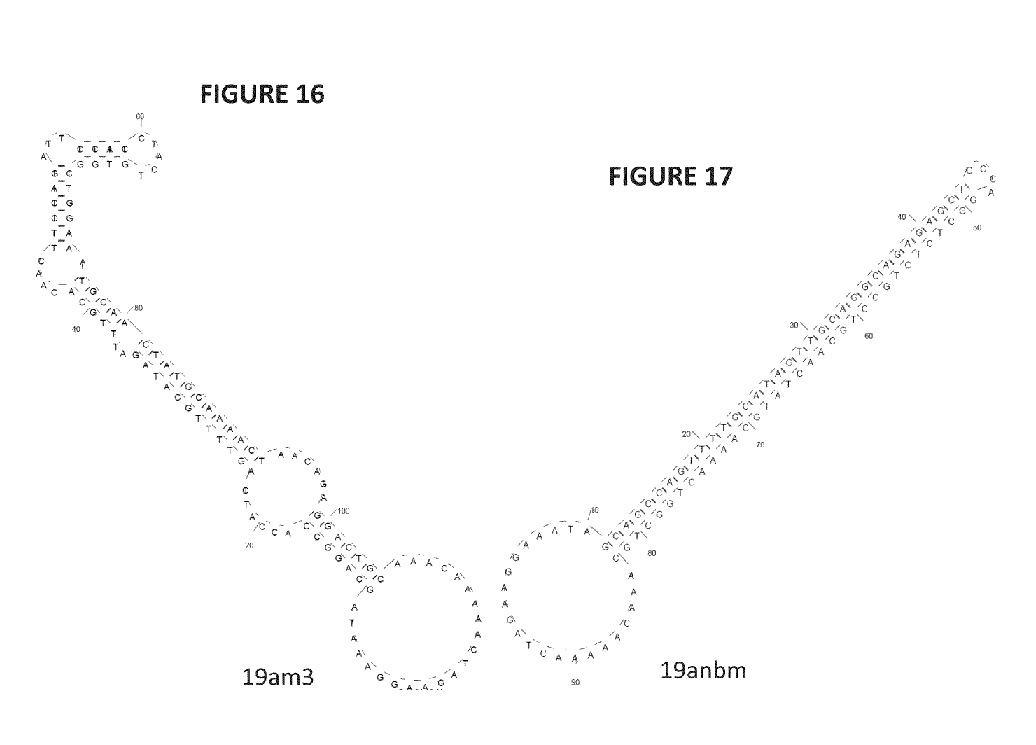

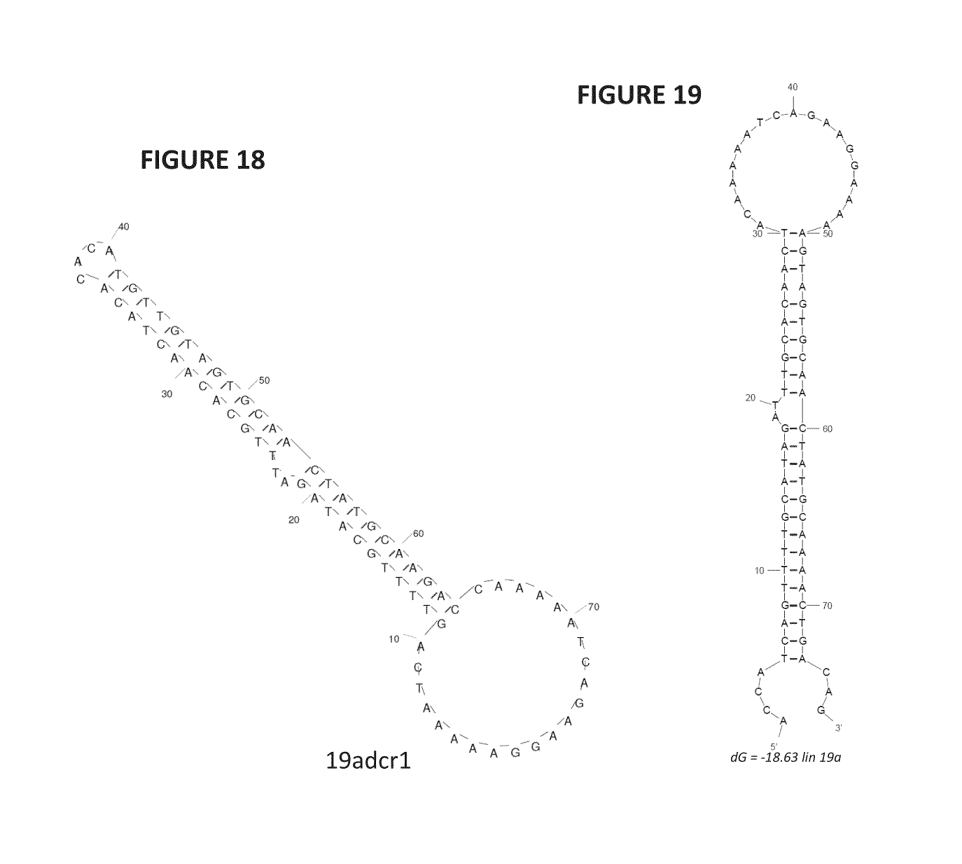

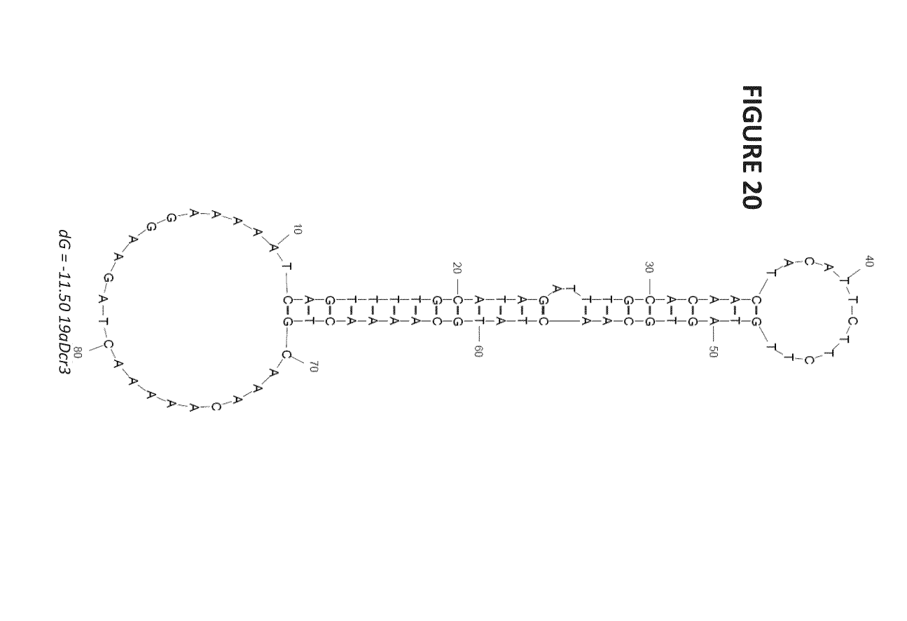

SEQ ID NOS: 1-12, upon circularization, produce single round transcript via
RNA Polymerase III.

| | | | |
|---|---|---|---|
| 1 | 19aTAR | CTCAGATCTGCCTGCAACTATGCAAAACTAACAGAGGACTGC | FIG. 5c |
| | (aka 19am) | AAACAAAAACTAGAAGGAAATAGCAGGCCACCATCAGTTTTG | |
| | | CATAGATTTGCACAACGGCAGAGAGCTCCCAGG | |
| 2 | 19am3 | GAAGGAAATAGCAGGCCACCATCAGTTTTGCATAGATTTGCA | FIG. 16 |
| | | CAACTTCCAGATTCCACCTACTGTGGCTGGAAATGCAACTAT | |
| | | GCAAAACTAACAGAGGACTGCAAACAAAAACTA | |
| 3 | 19adcr1 | GGAAAAATCAGTTTTGCATAGATTTGCACAACTACACACATG | FIG. 18 |
| | | TTGTAGTGCAACTATGCAAGACCAAAAATCAGAA | |
| 4 | 122 | TTGCCTAGCAGTAGCTATTTAGTGTGATAATGGCGTTTGATA | FIG. 5a, 5g |
| | (aka | GTTTAGACACAAACACCATTGTCACACTCCACAGCTCTGCTA | |
| | 122ENDO) | AGGAA | |
| 5 | 122TAR | AGGAAATAGCCTAGCAGTAGCTATTTAGTGTGATAATGGCGT | FIG. 5c |
| | (aka 122m) | TTGATAGGGCAGAGAGCTCCCAGGCTCAGATCTGCCCACAAA | |
| | | CACCATTGTCACACTCCACAGCTCTGCTAAGGAAACAAAA | |
| 6 | 122s | TTGCCCTATTTAGTGTGATAATGGCGTTTGATAGTTTAGACA | FIG. 5g |
| | | CAAACACCATTGTCACACTCCACAGGGAA | |
| 7 | 221 | ACGAACACAGAAATCTACATTGTATGCCAGGTTCATGAAACC | FIG. 6b |
| | | CAGCAGACAATGTAGCTGTTGCCTA | |
| 8 | 143 | AACTGACCAGAGATGCAGCACTGCACCTCTTCCTGAGCTACA | FIG. 6b |
| | | GTGCTTCATCTCAGAC | |
| 9 | 15a | AATCCACAAACCATTATGTGCTGCTACTTTGCAGCACAATAT | FIG. 6b |
| | | GGCCTGCACAA | |
| 10 | 21 | CAGACAGCCCATCGACTGGTGTTGACAGTCAACATCAGTCTG | FIG. 6b |
| | | ATAAGCTACCCGACA | |
| 11 | 19aDcr1 | GGAAAAATCAGTTTTGCATAGATTTGCACAACTACACACATG | FIG. 18 |
| | | TTGTAGTGCAACTATGCAAGACCAAAAATCAGAA | |
| 12 | 19aDcr3 | GAAGGAAAAATCAGTTTTGCATAGATTTGCACAACTACATTC | FIG. 20 |
| | | TTCTTGTAGTGCAACTATGCAAAACTGCAAACAAAAACTA | |

Linear sequences which were not transcribed or produced low levels of abortive
transcripts.

| | | | |
|---|---|---|---|
| 133 | lin19a | ACCATCAGTTTTGCATAGATTTGCACAACTACAAAAATCAGA | FIG. 19 |
| | | AGGAAAAAGTAGTGCAACTATGCAAAACTGACAG | |
| 14 | 122L | ACTCCACAGCTCTGCTAAGGAATTGCCTAGCAGTAGCTATTT | FIG. 24 |
| | | AGTGTGATAATGGCGTTTGATAGTTTAGACACAAACACCATT | |
| | | GTCAC | |

Other linear sequences

| | | | |
|---|---|---|---|
| 15 | Lin2-19a | CCTCAGTTTTGCATAGATTTGCACAACTAAAACAAAAACTAG | FIG. 23 |
| | | AAGGAAATAGTAGTGCAACTATGCAAAACTGAGG | |
| 16 | luc1309_wt | ACCGCCTGAAGTCTCTGATTAATACATCTGTGGCTTCACTAT | FIG. 21 |
| | DNA | TAATCAGAGACTTCAGGCGAAAAACTAGAAGGAAAA | |
| 17 | luc1309_ | ACCGCCTGAAGTCTCTGATTAATACATCTGTGGCTTCACTAT | FIG. 21 |
| | mut1DNA | TAATCAGAAACTTCAAGCGAAAAACTAGAAGGAAAA | |
| 18 | | AGACGGCAACACGTTTAGATACGTTTTGACTACCACCGGACG | FIG. 22 |
| | | ATAAAGGAAGATCAAAAACAAACGTCAGGAGACAATCAAAAC | |
| | | GTATCAACGTCCGTCT | |

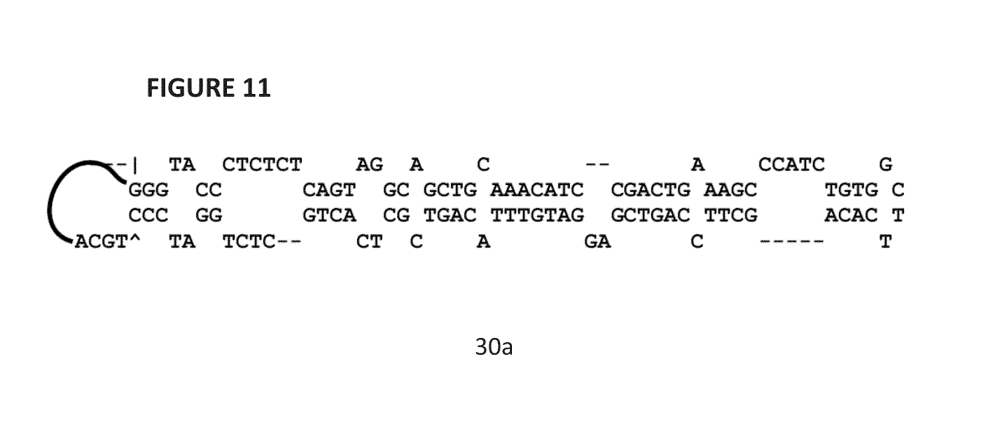

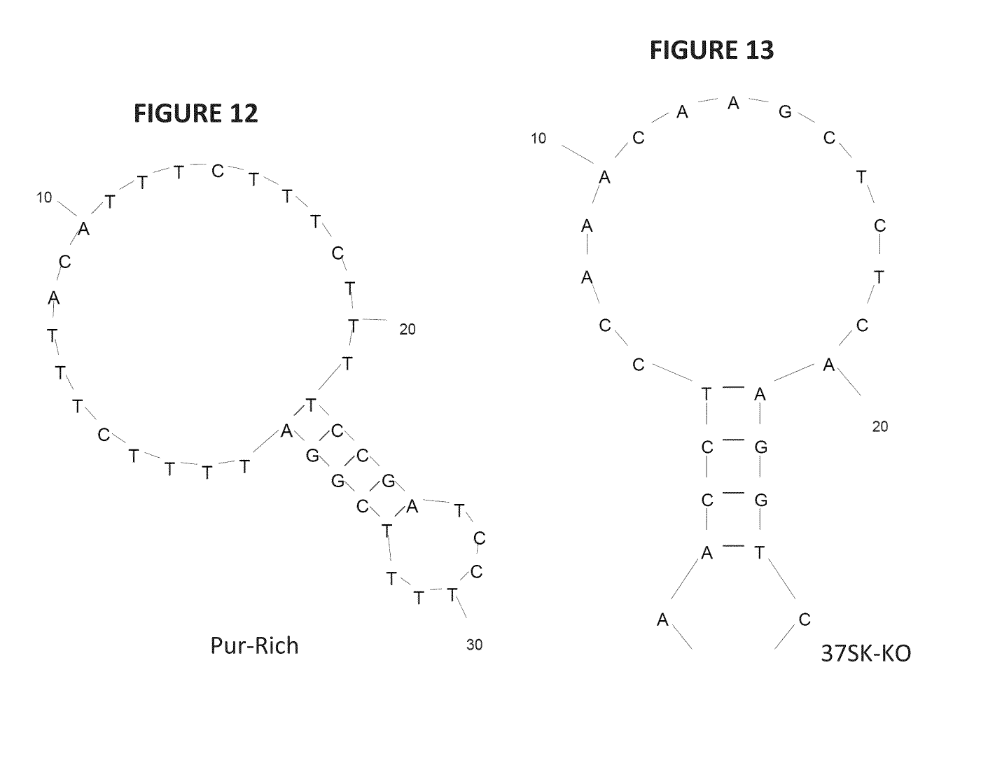

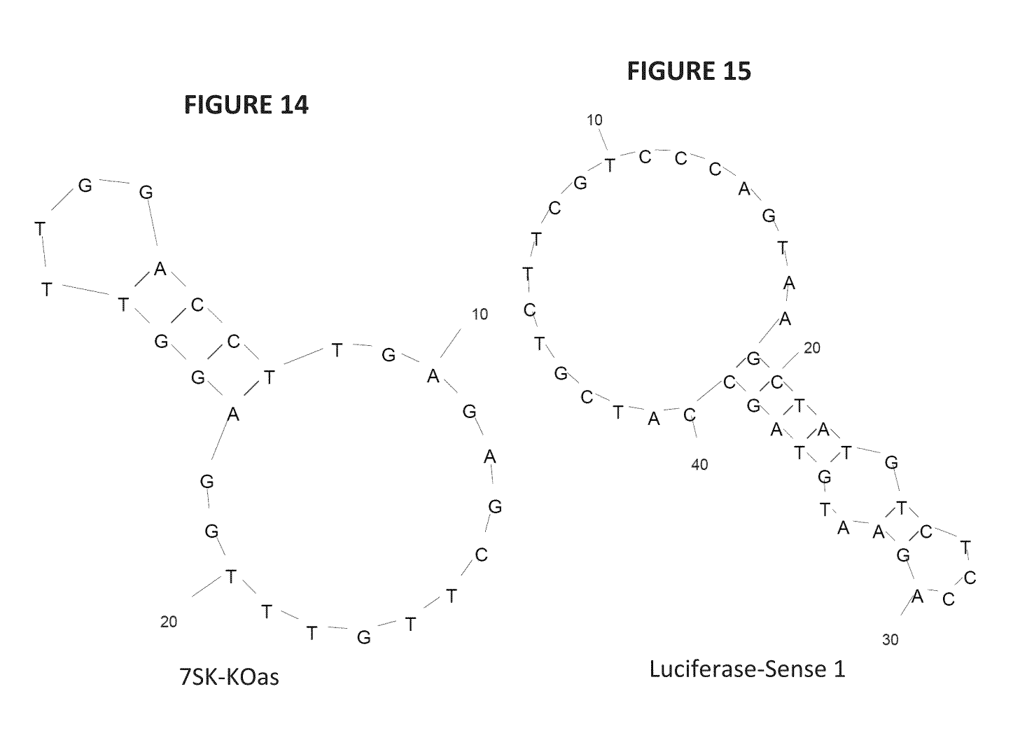

Circularization of the following sequences did not produce detectable single
round transcript.

| | | | |
|---|---|---|---|
| 19 | 19a | TAGCAGGCCACCATCAGTTTTGCATAGATTTGCACAACTACA | FIG. 5c, |
| | (aka | TTCTTCTTGTAGTGCAACTATGCAAAACTAACAGAGGACTGC | pyrimidine rich |
| | 19aENDO) | AA | larger loop, i.e. |
| | | | C/T-rich. |
| 20 | 19aRL | GAAGGAAATAGCAGGCCACCATCAGTTTTGCATAGATTTGCA | FIG. 7b, two |
| | aka | CAACTTCCCTATGACCTCGACTACGACTGGAAATGCAACTAT | larger loops |
| | 19a(Ex)TAR | GCAAAACTAACAGAGGACTGCAAACAAAAACTA | |
| 21 | 19anbm | GAAGGAAATAGCAGCCAGTTTTGCATAGTTGCAGGCAGAGAG | FIG. 17, |
| | | CTCCCAGGCTCTCTGCCTGCAACTATGCAAAACTGGCTGCAA | identical to |
| | | ACAAAAACTA | 19aTAR except all |
| | | | internal bulge |
| | | | loops removed, |
| | | | resulting in |
| | | | perfectly base- |
| | | | paired stem. |
| 22 | RANDC1 | ATGATCTAAAAACGGTGCTGTGTATGTCTGCTTTGATCAACC | FIG. 6d, random |
| | | TCTAATAGCTCGTATGATAGTGCAGCCGCTGGTGATCACTGT | sequence, doesn't |
| | | GCGAATACGGGTTGTAGCAATGTTCGTCTGAGT | encode RNA with |
| | | | stem loop |
| | | | secondary |
| | | | structure. |
| 23 | 30a | GGGTACCCTCTCTCAGTAGGCAGCTGCAAACATCCGACTGAA | FIG. 11, no |
| | | AGCCCATCTGTGGCTTCACAGCTTCCAGTCGAGGATGTTTAC | larger loop; one |
| | | AGTCGCTCACTGCTCTGGATCCCTGCA | large internal |
| | | | bulge but it's |
| | | | pyrimidine-rich. |
| 24 | Pyr-rich | TTTCTTTACATTTCTTTCTTTTCCGATCCTTTTCGGAT | FIG. 12, not a |
| | | | stem loop |
| | | | secondary |
| | | | structure, and |
| | | | its one larger |
| | | | loop is |
| | | | pyrimidine rich |
| 25 | 7SK-KO | AACCTCCAAACAAGCTCTCAAGGTCCA | FIG. 13, not a |
| | | | stem loop |
| | | | structure |
| 26 | 7SK-KOas | TGGACCTTGAGAGCTTGTTTGGAGGTT | FIG. 14, not a |
| | | | stem loop |
| | | | structure |
| 27 | Luciferase- | TCGTCTTCGTCCCAGTAAGCTATGTCTCCAGAATGTAGCCA | FIG. 15, not a |
| | sense1 | | stem loop |
| | | | structure |

RNA transcripts from coligo or linear templates

| | | | |
|---|---|---|---|
| 28 | Predicted | UUGCAGUCCUCUGUUAGUUUUGCAUAGUUGCACUACAAGA | FIG. 3b |
| | but | AGAAUGUAGUUGUGCAAAUCUAUGCAAAACUGAUGGUGGC | |
| | undetected | CUGCUA | |
| | RNA | | |
| | transcript | | |
| | from 19a | | |
| 29 | 122 | AUCAAACGCCAUUAUCACACUAAAUAGCUACUGCUAGGCA | FIG. 3b, 5a |
| | transcript | AUUCCUUAGCAGAGCUGUGGAGUGUGACAAUGGUGUUUGU | |
| | | GUC | |
| 30 | RNA | GUUUGCAGUCCUCUGUUAGUUUUGCAUAGUUGCAGGCAGA | FIG. 3b |
| | transcript | UCUGAGCCUGGGAGCUCUCUGCCGUUGUGCAAAUCUAUGC | |
| | from 19aTAR | AAAACUGAUGGUGGCCUGCUAUUUCCUUCUAG | |
| | (19am) | | |
| 31 | 122m (aka | UUUUUGUUUCCUUAGCAGAGCUGUGGAGUGUGACAAUGGU | FIG. 3b |
| | 122 TAR) | GUUUGUGGGCAGAUCUGAGCCUGGGAGCUCUCUGCCCUAU | |
| | predicted | CAAACGCCAUUAUCACACUAAAUAGCUACUGCUAGGCUAU | |
| | transcript | UUCC | |
| 32 | RNA | GUUUGCAGUCCUCUGUUAGUUUUGCAUAGUUGCAGGCAGA | FIG. 5e |
| | transcript | UCUGAGCCUGGGAGCUCUCUGCCGUUGUGCAAAUCUAUGC | |
| | from coligo | AAAACUGAUGGUGGCCUGCUAUUUCCUUCUAG | |
| | 19aTAR | | |
| 33 | RNA | GUUUGCAGUCCUCUGUUAGUUUUGCAUAGUUGCAGGCAGA | FIG. 5e |
| | transcript | UCUGAGCCUGGGAGCUCUCUGCCGUUGUGCAAAUCUAUGC | |
| | from coligo | AAAACUGAUGGUGGCCUGCUAUUUCCUUC | |
| | 19aTAR | | |
| 34 | RNA | GUUUGCAGUCCUCUGUUAGUUUUGCAUAGUUGCAGGCAGA | FIG. 5e |
| | transcript | UCUGAGCCUGGGAGCUCUCUGCCGUUGUGCAAAUCUAUGC | |
| | from coligo | AAAACUGAUGGUGGCCUGCUAUUUCCUUCUAGU | |
| | 19aTAR | | |
| 35 | RNA | GUUUGCAGUCCUCUGUUAGUUUUGCAUAGUUGCAGGCAGA | FIG. 5e |
| | transcript | UCUGAGCCUGGGAGCUCUCUGCCGUUGUGCAAAUCUAUGC | |
| | from coligo | AAAACUGAUGGUGGCCUGCUAUUUCCUUCUAGUU | |
| | 19aTAR | | |
| 36 | RNA | GUUUGCAGUCCUCUGUUAGUUUUGCAUAGUUGCAGGCAGA | FIG. 5e |
| | transcript | UCUGAGCCUGGGAGCUCUCUGCCGUUGUGCAAAUCUAUGC | |
| | from coligo | AAAACUGAUGGUGGCCUGCUAUUUCCUUCUAGUUU | |
| | 19aTAR | | |
| 37 | RNA | GUUUGCAGUCCUCUGUUAGUUUUGCAUAGUUGCAGGCAGA | FIG. 5e |
| | transcript | UCUGAGCCUGGGAGCUCUCUGCCGUUGUGCAAAUCUAUGC | |
| | from coligo | AAAACUGAUGGUGGCCUGCUAUUUCCUUCUAGUUUUUGUU | |
| | 19aTAR | U | |
| 38 | RNA | AUCAAACGCCAUUAUCACACUAAAUAGCUACUGCUAGGCA | FIG. 5f |
| | transcript | AUUCCUUAGCAGAGCUGUGGAGUGUGACAAUGGUGUUUGU | |
| | from coligo | GUC | |
| | 122 or | | |
| | linear 122L | | |
| 39 | RNA | AUCAAACGCCAUUAUCACACUAAAUAGCUACUGCUAGGCA | FIG. 5f |
| | transcript | AUUCCUUAGCAGAGCUGUGGAGUGUGACAAUGGUGUUUGU | |
| | from coligo | GUCU | |
| | 122 | | |
| 40 | RNA | AUCAAACGCCAUUAUCACACUAAAUAGCUACUGCUAGGCA | FIG. 5f |
| | transcript | AUUCCUUAGCAGAGCUGUGGAGUGUGACAAUGGUGUUUGU | |
| | from coligo | GU | |
| | 122 | | |
| 41 | Predicted | AUCAAACGCCAUUAUCACACUAAAUAGGGCAAUUCCCUGU | FIG. 5g |
| | transcript | GGAGUGUGACAAUGGUGUUUGUGUC | |
| | of 122s | | |
| 42 | Predicted | CGCCUGAAGUCUCUGAUUAAUAGUGAAGCCACAGAUGUAU | FIG. 21 |
| | but | UAAUCAGAGACUUCAGGCGGU | |
| | undetected | | |
| | transcript | | |
| | from | | |
| | luc1309_wt | | |
| 43 | luc1309_GU2, | CGCUUGAAGUUUCUGAUUAAUAGUGAAGCCACAGAUGUAU | FIG. 21 |
| | predicted | UAAUCAGAGACUUCAGGCGGU | |
| | transcript | | |
| | from | | |
| | luc1309_mut1 | | |
| | DNA | | |
| 44 | Predicted | GUUUGCAGUCCUCUGUUAGUUUUGCAUAGUUGCAGGCAGA | FIG. 22 |
| | run-off | | |
| | transcript | | |
| | from the | | |
| | linear | | |
| | template of | | |
| | FIG. 22 | | |
| 45 | Predicted | GUUUUAGUUGUGCAAAUCUAUGCAAAACUGAGG | FIG. 23 |
| | transcript | | |
| | from lin2- | | |
| | 19a | | |

Other sequences

| | | |
|---|---|---|
| 46 | 19am-1 | 5′pGAAGGAAATAGCAGGCCACCATCAGTTTTGCATAGAT |
| | | TTGCACAACGGCAGAGAGCTCCCAGG |
| 47 | 19am-2 | 5′pCTCAGATCTGCCTGCAACTATGCAAAACTAACAGAGG |
| | | ACTGCAAACAAAAACTA |
| 48 | splint 19am | 5′CAGGCAGATCTGAGCCTGGGAGCTCTCTGC |
| 49 | 122m-1 | 5′pAGGAAATAGCCTAGCAGTAGCTATTTAGTGTGATAAT |
| | | GGCGTTTGATAGGGCAGAGAGCTCCCAG |
| 50 | 122m-2 | 5′pGCTCAGATCTGCCCACAAACACCATTGTCACACTCCA |
| | | CAGCTCTGCTAAGGAAACAAAA, |
| 51 | splint 122m | 5′TGGGCAGATCTGAGCCTGGGAGCTCTCTGCC |
| 52 | Oligo probe | 5′TGGACCTTGAGAGCTTGTTTGGAGGTT |
| 53 | Oligo probe | 5′AGCTCCTATTCCATCTCCCTGCTC |
| 54 | Oligo probe | TCGTCTTCGTCCCAGTAAGCTATGTCTCCAGAATGTAGCC |
| | | A |
| 55 | Oligo | CGGCAGAGAGCTCCCAGGCTCAGATCTGCC |
| 56 | Universal | 5′rAppCTGTAGGCACCATCAAT-NH2-3′ |
| | miRNA | |
| | Cloning | |
| | Linker (NEB) | |
| 57 | 5′adaptor | 5′GCUGAUGGCGAUGAAUGAACACUGCGUUUGCUGGCUUU |
| | (Ambion RLM- | GAUGAAA |
| | RACE kit) | |
| 58 | MiRclon3NotI | 5′AGCGGCCGCCTGCAGATTGATGGTGCCTACAG |
| | (IDT) | |
| 59 | 5′ outer | 5′GCTGATGGCGATGAATGAACACTG |
| | primer | |
| | (Ambion RLM- | |
| | RACE kit) | |
| 60 | 5′ inner | 5′CGCGGATCCGAACACTGCGTTTGCTGGCTTTGATG |
| | primer | |
| | (Ambion RLM- | |
| | RACE kit) | |
| 61 | 5end122-5 | 5′CAGCGGCCGCGAATTCTATTTAGTGTGATAA |
| | (IDT) | |
| 62 | 5end122-6 | 5′AAGCGGCCGCGAATTCTTGCCTAGCAGTAGC |
| 63 | 5end122-2 | 5′GTGCGGCCGCGAATTCATTGTCACACTCCA |
| | (IDT) | |
| 64 | 19aT-fwd | 5′GCATGGATCCGAAGGAAATAGCAGGCCA |
| | (IDT) | |
| 65 | 3′ RACE | 5′GCGAGCACAGAATTAATACGACTCACTATAGGT12(A, |
| | adapter | G or C)N-3′, “N” represents amino |
| | | blocking group. |
| 66 | 3′ RACE | 5′CGCGGATCCGAATTAATACGACTCACTATAGG-3′ |
| | Inner Primer | |
| 67 | 122aE3end2 | 5′TAGGATCCTCACAAACGCCATTATCACACT-3′ |
| 68 | RNAP III | AAAAA |
| | termination | |
| | signal | |
| 69 | RNAP III | AAACA |
| | termination | |
| | signal | |
| 70 | RNAP III | AAAAA |
| | termination | |
| | signal | |
| 71 | a −10 | TATAAT |
| | element | |
| 72 | a −35 | TTGACAT |
| | element | |
| 73 | TATA box | TATAAA |

What is claimed is:

1. A synthetic single-stranded DNA molecule, characterized by a secondary structure which comprises a stem-loop structure, wherein the stem includes at least one bulge of imperfect base pairing, and the loop is purine-rich with at least one pyrimidine approximate to the 3′ end of the stem and composed of 10-25 nucleotides.

2. The DNA molecule of claim 1, wherein at least 33% of the nucleotides in the loop are A's.

3. The DNA molecule of claim 1, wherein more than 50% of the nucleotides in the loop are A's or G's.

4. The DNA molecule of claim 1, wherein C's and T's in the loop in combination do not exceed 33%.

5. The DNA molecule of claim 1, wherein said bulge is composed of 1-6 pairs of unpaired bases.

6. The DNA molecule of claim 5, wherein said bulge is within 6 nucleotides from the stem-loop junction.

7. The DNA molecule of claim 1, wherein the secondary structure further comprises a second loop which is at the opposite end of the stem in relation to the purine-rich loop, wherein the second loop is composed of 3-9 nucleotides.

8. The DNA molecule of claim 7, said DNA molecule being a circular DNA molecule.

9. The DNA molecule of claim 8, wherein the circular DNA molecule includes a RNA polymerase III transcription termination sequence at the junction of the purine-rich loop and the 5′ end of the stem, or wholly contained in the purine-rich loop.

10. The DNA molecule of claim 7, said DNA molecule being a linear DNA molecule, wherein the point of discontinuity of the DNA strand is located near the middle of the stem, between the two terminal loops, in a fully base-paired region.

11. The DNA molecule of claim 1, said DNA molecule being a linear DNA molecule, wherein the point of discontinuity of the DNA strand is located at the end of the stem, opposite to the purine-rich loop.

12. The DNA molecule of claim 1, wherein the purine-rich loop includes a non-natural nucleotide mimics.

13. The DNA molecule of claim 1, wherein the purine-rich loop includes a DNA aptamer sequence which facilitates cell penetration.

14. The DNA molecule of claim 1, wherein the purine-rich loop is catenated with a DNA circle.

15. The DNA molecule of claim 1, said molecule being composed of fewer than 250 nucleotides.

16. A method for producing small RNA molecules, comprising:
i) providing a synthetic single-stranded DNA molecule according to claim 1; and
ii) exposing said synthetic single-stranded DNA molecule to a mammalian RNA polymerase III activity to initiate transcription; thereby producing small RNA molecules.

17. The method of claim 16, wherein said small RNA molecules are selected from the group consisting of minimized primary-microRNA, pre-shRNA, shRNA, pre-microRNA, microRNA (miRNA), small interfering RNA (siRNA), aptamers, antisense RNA, ribozymes, antisense miRNA (i.e. antagomirs), tRNA and small ribosomal RNA.

18. The method of claim 16, wherein said mammalian RNA polymerase III is human RNA polymerase III.

19. The method of claim 16, wherein the single-stranded DNA molecule is exposed to said mammalian RNA polymerase III activity in vitro.

20. The method of claim 16, wherein the single-stranded DNA molecule is exposed to said mammalian RNA polymerase III activity in situ.

21. The method of claim 16, wherein the single-stranded DNA molecule is exposed to said mammalian RNA polymerase III activity in vivo in a mammal.
